(19) United States
(12) Patent Application Publication
(10) Pub. No.: US 2024/0101690 A1
(43) Pub. Date: Mar. 28, 2024

(54) TREATMENT OF SKIN DISEASES OR DISORDERS BY DELIVERY OF ANTI-OSMRBeta ANTIBODY

(71) Applicant: Kiniksa Pharmaceuticals, Ltd., Hamilton (BM)

(72) Inventors: John Paolini, Lexington, MA (US); Rohan Gandhi, Lexington, MA (US); Zamaneh Mikhak, Lexington, MA (US)

(21) Appl. No.: 18/193,393

(22) Filed: Mar. 30, 2023

Related U.S. Application Data
(63) Continuation of application No. 17/050,261, filed on Oct. 23, 2020, now abandoned.
(60) Provisional application No. 62/662,607, filed on Apr. 25, 2018.
(60) Provisional application No. 62/718,324, filed on Aug. 13, 2018.
(60) Provisional application No. 62/765,033, filed on Aug. 16, 2018.
(60) Provisional application No. 62/731,618, filed on Sep. 14, 2018.
(60) Provisional application No. 62/757,047, filed on Nov. 7, 2018.
(60) Provisional application No. 62/775,350, filed on Dec. 4, 2018.
(60) Provisional application No. 62/789,434, filed on Jan. 7, 2019.
(60) Provisional application No. 62/794,356, filed on Jan. 18, 2019.

(51) Int. Cl. C07K 16/28; A61K 39/395; A61P 17/04; G01N 33/53

(52) U.S. Cl. CPC C07K 16/2866; A61K 39/3955; A61P 17/04; G01N 33/53; A61P 17/00

(57) ABSTRACT

The present invention provides, among other things, methods of treating pruritic or inflammatory skin diseases or in disorders, or pruritus associated with a disease or disorder, with an anti-OSMRβ antibody, including methods of treating pruritus, in associated with atopic dermatitis, chronic kidney disease-associated pruritus, uremic pruritus or prurigo nodularis, chronic idiopathic pruritus, chronic idiopathic urticaria, chronic spontaneous urticaria, cutaneous amyloidosis, lichen simplex chronicus, plaque psoriasis, lichens planus, inflammatory ichthyosis, mastocytosis and bullous pemphigoid, comprising a step of administering to a subject in need of treatment an anti-OSMRβ antibody at a therapeutically effective dose and an administration interval for a treatment period sufficient to improve, stabilize or reduce one or more symptoms of the disease or disorder relative to a control.

### CROSS-REFERENCE TO RELATED APPLICATIONS

This application claims benefit of, and priority to, U.S. Provisional Patent Application Ser. No. 62/662,607, filed on Apr. 25, 2018; 62/718,324, filed on Aug. 13, 2018; 62/731,618, filed on Sep. 14, 2018; 62/757,047, filed on Nov. 7, 2018; 62/765,033, filed on Aug. 16, 2018; 62/775,350, filed on Dec. 4, 2018; 62/789,434, filed on Jan. 7, 2019; and 62/794,356, filed on Jan. 18, 2019, the contents of each of which are incorporated herein.

### INCORPORATION-BY-REFERENCE OF SEQUENCE LISTING

The contents of the text file named “KPL-003WO_ST25. txt” which was created on Apr. 25, 2019 and is 17.2 KB in size, are hereby incorporated by reference in its entirety.

### BACKGROUND

Atopic dermatitis is a chronic inflammatory skin disorder primarily characterized by extreme itching, leading to scratching and rubbing that in turn results in the typical lesions of eczema. Various diseases and disorders are accompanied by pruritus (itch). For example, patients with renal failure, usually end-stage renal disease (ESRD), commonly are afflicted by severe pruritus (uremic pruritus). Prurigo nodularis (PN), also known as nodular prurigo is a skin disease characterized by itchy nodules that usually appear in the arms and legs. Patients often present with multiple excoriating lesions caused by scratching. Severe pruritus is a seriously debilitating condition. The uncomfortable and often painful symptoms associated with atopic dermatitis and uremic pruritus include itching, swelling, redness, blisters, crusting, ulceration, pain, scaling, cracking, hair loss, scarring, or oozing of fluid involving the skin, eye, or mucosal membranes. Other debilitating skin conditions that are accompanied by pruritus include Chronic Idiopathic Pruritus, Chronic Idiopathic Urticaria, Chronic Spontaneous Urticaria, Cutaneous Amyloidosis, Lichen Simplex Chronicus, Plaque Psoriasis, Lichens Planus, Inflammatory Ichthyosis, Mastocytosis and Bullous Pemphigoid.

The need to control pruritus has led to a search for therapeutic agents that are both safe and effective. Corticosteroids, when administered systemically, are effective in this regard but are associated with significant and potentially dangerous side effects. Topically applied corticosteroids have some efficacy in treating these conditions, but are only partially effective in many instances and have their own significant side effects. Other agents with partial utility for treating some of the dermatitis and uremic pruritus.

### SUMMARY OF THE INVENTION

The present invention provides, among other things, methods of treating pruritic or inflammatory skin diseases or disorders, or pruritus associated with a disease or disorder, with an anti-OSMRP antibody. In particular, the present invention provides methods for treating prurigo nodularis, atopic dermatitis, uremic pruritus, and pruritus associated with Chronic Idiopathic Pruritus, Chronic Idiopathic Urticaria, Chronic Spontaneous Urticaria, Cutaneous Amyloidosis, Lichen Simplex Chronicus, Plaque Psoriasis, Lichen Planus, Inflammatory Ichthyosis, Mastocytosis or Bullous Pemphigoid, to name but a few.

In one aspect, the present invention provides methods for treating prurigo nodularis, comprising a step of administering to a subject in need of treatment an anti-OSMRP antibody at a therapeutically effective dose and an administration interval for a treatment period sufficient to improve, stabilize or reduce one or more symptoms of prurigo nodularis relative to a control. In some embodiments, the subject presents with pruritic hyperkeratotic nodules.

In some embodiments, the prurigo nodularis is idiopathic. In some embodiments, the prurigo nodularis is not associated with any other underlying co-morbidities.

In some embodiments, the prurigo nodularis is associated with one or more underlying co-morbidities.

In some embodiments, IL-31 expression level is elevated in the subject relative to a control. In some embodiments, IL-31 expression level is not elevated in the subject relative to a control. In some embodiments, IL-31 expression level in a portion of the subject's skin affected by a pruritic disease or condition is approximately the same as the IL-31 expression level in (i) a portion of the subject's skin that is unaffected by the pruritic disease or condition, or (ii) a portion of normal skin from a healthy subject, who is not diagnosed with a pruritic disease or condition. In some embodiments, IL-31Rα expression level is elevated in the subject relative to a control. In some embodiments, OSM expression level is elevated in the subject relative to a control. In some embodiments, OSMRβ expression level is elevated in the subject relative to a control. In some embodiments, OSMRβ expression level is not elevated in the subject relative to a control. In some embodiments, OSMRβ expression level in a portion of the subject's skin affected by a pruritic disease or condition is approximately the same as the OSMRβ expression level in (i) a portion of the subject's skin that is unaffected by the pruritic disease or condition, or (ii) a portion of normal skin from a healthy subject, who is not diagnosed with a pruritic disease or condition.

In some embodiments, the levels of any one of IL-31, IL-31Rα, OSM and OSMRβ in the subject is determined via skin biopsy from hyperkeratotic nodules. In some embodiments, the control is a healthy subject who is not diagnosed with a pruritic disease.

In some embodiments, the subject in need of treatment has a score on a pruritus NRS greater than or equal to 5

In some embodiments, the subject in need of treatment has a score on a pruritus NRS greater than or equal to 7.

In some embodiments, the subject in need of treatment has elevated MCP-1/CCL2 levels in comparison to a control subject.

In some embodiments, treating results in a reduction of MCP-1/CCL2 levels in the subject.

In some embodiments, treating results in a reduction of MCP-1/CCL2 levels in the subject equivalent to levels in a healthy subject. In some embodiments, treating results in a reduction of MCP-1/CCL2 levels in the subject equivalent to levels in a control subject who does not have the disease.

In another aspect, the invention provides methods of treating atopic dermatitis comprising a step of administering to a subject in need of treatment an anti-OSMRβ antibody at a therapeutically effective dose and an administration interval for a treatment period sufficient to improve, stabilize or reduce one or more symptoms of atopic dermatitis relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection. In some embodiments, the subcutaneous administration is through a subcutaneous pump. In some embodiments, the therapeutically effective dose comprises an initial bolus or loading dose. In some embodiments, the therapeutically effective dose comprises a maintenance dose. In some embodiments, the therapeutically effective dose comprises an initial bolus or loading dose, followed by at least one maintenance dose. In some embodiments, the therapeutically effective dose is an initial bolus or loading dose, and wherein the method further comprises administering at least one maintenance dose. In some embodiments, the step of administering comprises an initial bolus or loading dose, followed by at least one maintenance dose. In some embodiments, the initial bolus or loading dose is greater than the at least one maintenance dose. In some embodiments, the initial bolus or loading dose is at least one fold, two fold, three fold, four fold or five fold greater in dosage than the dosage of the at least one maintenance dose. In some embodiments, the initial bolus or loading dose is two fold greater in dosage than the dosage of the at least one maintenance dose.

In some embodiments, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

In some embodiments, the one or more symptoms of atopic dermatitis are assessed by an Investigators' Global Assessment (IGA) of atopic dermatitis. In some embodiments, the one or more symptoms of atopic dermatitis are assessed by an Eczema Area and Severity Index (EAST). In some embodiments, the one or more symptoms of atopic dermatitis are assessed by SCORing Atopic Dermatitis. In some embodiments, the one or more symptoms of atopic dermatitis are assessed by atopic dermatitis area photographs. In some embodiments, the one or more symptoms of atopic dermatitis are assessed by Body Surface Area Involvement (BSA) of Atopic Dermatitis. In some embodiments, the one or more symptoms of atopic dermatitis are assessed by a Dermatology Life Quality Index (DLQI). In some embodiments, the one or more symptoms of atopic dermatitis are assessed by a Hospital Anxiety and Depression Scale (HADS). In some embodiments, the one or more symptoms of atopic dermatitis, such as sleep quality and sleep quantity, are assessed by actigraphy. In some embodiments, the one or more symptoms of atopic dermatitis are assessed by a quantitative numerical pruritus scale, e.g., Pruritus Numerical Rating Scale (NRS), Visual Analogue Scale (VAS) or Verbal Rating Scale (VRS). Pruritus VAS is a component of SCORAD and reflects the average pruritus experienced in the prior 3 day period, where, 0=no itch and 10=worst imaginable itch. In some embodiments, the administration of an anti-OSMRβ antibody results in a statistically-significant drop on a quantitative numerical pruritus scale. In some embodiments, the administration of an anti-OSMRβ antibody results in at least one of an improvement in the subject's quality of life, quality of sleep and quantity of sleep. In some embodiments, sleep loss is assessed by sleep-loss VAS, a component of SCORAD, at designated study visits. Sleep loss VAS reflects the average level of sleeplessness experienced in the prior 3-night period. 0=no sleeplessness, 10=worst imaginable sleeplessness.

Typically, a control is indicative of the one or more disease parameters of atopic dermatitis without the treatment. In some embodiments, a control is the respective value of a disease parameter of a subject with comparable disease status, but without treatment. In some embodiments, a control is the respective value of a disease parameter of a subject with comparable disease status but treated with a placebo. In some embodiments, a control is the respective value of a disease parameter of a subject prior to treatment (also referred to as baseline). In some embodiments, a control is a reference value indicative of a disease parameter without treatment based on collective knowledge, or historical data.

In some embodiments, the one or more symptoms of atopic dermatitis in the subject before the treatment comprises a score on a pruritus NRS greater than or equal to 4, or an equivalent assessment using a quantitative numerical pruritus scale. In some embodiments, the one or more symptoms of atopic dermatitis in the subject before the treatment comprises a score on a pruritus NRS greater than or equal to 7, or an equivalent assessment using a quantitative numerical pruritus scale. In some embodiments, the subject in need of treatment has been diagnosed with moderate to severe atopic dermatitis, wherein moderate to severe atopic dermatitis comprises IGA of 3 or 4 and BSA involvement of approximately 10% or more. In some embodiments, the control is indicative of the one or more symptoms of atopic dermatitis in a control subject with the same disease status without treatment. In some embodiments, the control is indicative of the one or more symptoms of atopic dermatitis in a control subject with the same disease status that was administered a placebo.

In some embodiments, the administration results in no serious adverse effects in the subject. In some embodiments, the administration does not result in one or more of peripheral edema, exacerbation of atopic dermatitis, nasopharyngitis, upper respiratory tract infections, increased creatine phosphokinase, conjunctivitis, blepharitis, oral herpes, keratitis, eye pruritus, other herpes simplex virus infection, and dry eye.

In a further aspect, the present invention provides methods of treating uremic pruritus, comprising a step of administering to a subject in need of treatment an anti-OSMRβ antibody at a therapeutically effective dose and an administration interval for a treatment period sufficient to improve, stabilize or reduce one or more symptoms of uremic pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection. In some embodiments, the subcutaneous administration is through a subcutaneous pump. In some embodiments, the step of administering comprises an initial bolus or loading dose, followed by at least one maintenance dose. In some embodiments, the initial bolus or loading dose is greater than the at least one maintenance dose. In some embodiments, the initial bolus or loading dose is at least one fold, two fold, three fold, four fold or five fold greater in dosage than the dosage of the at least one maintenance dose. In some embodiments, the initial bolus or loading dose is two fold greater in dosage than the dosage of the at least one maintenance dose. As used herein, an initial bolus or loading dose, an initial loading dose and an initial dose are terms used interchangeably.

In some embodiments, the one or more symptoms of uremic pruritus are assessed by a Dermatology Life Quality Index (DLQI). In some embodiments, the one or more symptoms of uremic pruritus are assessed by a Hospital Anxiety and Depression Scale (HADS). In some embodiments, the one or more symptoms of atopic dermatitis, such as sleep quality and sleep quantity, are assessed by actigraphy. In some embodiments, the administration of an anti-OSMRB antibody results in at least one of an improvement in the subject's quality of life, quality of sleep and quantity of sleep.

In some embodiments, the control is indicative of the one or more symptoms of uremic pruritus in the subject before the treatment. In some embodiments, the one or more symptoms of uremic pruritus in the subject before the treatment comprises a score on a pruritus NRS greater than or equal to 5, or an equivalent assessment using a quantitative numerical pruritus scale. In some embodiments, the one or more symptoms of uremic pruritus in the subject before the treatment comprises a score on a pruritus NRS greater than or equal to 7, or an equivalent assessment using a quantitative numerical pruritus scale. In some embodiments, the subject in need of treatment has end stage renal disease. In some embodiments, the subject in need of treatment is undergoing a hemodialysis regimen of at least one time-per-week. In some embodiments, the subject in need of treatment is undergoing a three-times-per-week hemodialysis regimen. In some embodiments, the three-times-per-week hemodialysis regimen has been stable for at least three months. In some embodiments, the control is indicative of the one or more symptoms of uremic pruritus in a control subject with the same disease status without treatment. In some embodiments, the control is indicative of the one or more symptoms of uremic pruritus in a control subject with the same disease status that was administered a placebo.

In still another aspect, the present invention provides methods and compositions for treating pruritus in a subject suffering from a kidney disease. In some embodiments, the subject suffers from chronic kidney disease. In some embodiments, the subject having chronic kidney disease has not undergone dialysis. In some embodiments, the present invention provides a method and compositions for use in treating chronic kidney disease-associated pruritus in predialysis subjects.

In some embodiments, the method comprises a step of administering to a subject in need of treatment an anti-OSMRβ antibody at a therapeutically effective dose and an administration interval for a treatment period sufficient to improve, stabilize or reduce one or more symptoms of chronic kidney disease associated pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection. In some embodiments, the subcutaneous administration is through a subcutaneous pump. In some embodiments, the step of administering comprises an initial bolus or loading dose, followed by at least one maintenance dose.

In some embodiments, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

In some embodiments, the treatment period is for as long as the subject is on hemodialysis. In some embodiments, the step of administering occurs one day before the subject undergoes hemodialysis. In other embodiments, the step of administering occurs during hemodialysis. In other embodiments, the step of administering occurs on the day of hemodialysis, after hemodialysis has been completed. In other embodiments, the step of administering occurs within one day after hemodialysis.

In some embodiments, the one or more symptoms of uremic pruritus are assessed by a quantitative numerical pruritus scale, e.g., Pruritus Numerical Rating Scale (NRS), Visual Analogue Scale (VAS) or Verbal Rating Scale (VRS). In some embodiments, the administration of an anti-OSMRβ antibody results in a statistically-significant drop on a quantitative numerical pruritus scale.

In yet another aspect, the present invention provides a method for treating pruritus in a subject having a disease or a condition selected from Chronic Idiopathic Pruritus (CIP), Chronic Idiopathic Urticaria (CIU), Chronic Spontaneous Urticaria (CSU), Cutaneous Amyloidosis (CA), Lichen Simplex Chronicus (LSC), Plaque Psoriasis (PPs), Lichens Planus (LP), Inflammatory Ichthyosis (II), Mastocytosis (MA) and Bullous Pemphigoid (BP). In some embodiments, the method comprising a step of administering to the subject in need of treatment an anti-OSMRβ antibody at a therapeutically effective dose and an administration interval for a treatment period sufficient to improve, stabilize or reduce pruritus relative to a control.

In some embodiments, the subject has CIP. In some embodiments, the subject has CSU. In some embodiments, the subject has CIU. In some embodiments, the subject has CA. In some embodiments, the subject has LSC. In some embodiments, the subject has PPs. In some embodiments, the subject has LP. In some embodiments, the subject has II. In some embodiments, the subject has MA. In some embodiments, the subject has BP.

In some embodiments, the present invention provides a method of treating CIU, the method comprising administering to the subject in need of treatment an anti-OSMRβ antibody at a therapeutically effective dose and an administration interval for a treatment period sufficient to improve, stabilize or reduce urticaria relative to a control.

In some embodiments, the administration results in no serious adverse effects in the subject. In some embodiments, the administration does not result in one or more of peripheral edema, nasopharyngitis, upper respiratory tract infections, increased creatine phosphokinase, conjunctivitis, blepharitis, oral herpes, keratitis, eye pruritus, other herpes simplex virus infection, and dry eye.

In various aspects and embodiments described herein, the anti-OSMRB antibody comprises a light chain complementary-determining region 1 (LCDR1) defined by SEQ ID NO: 8, a light chain complementary-determining region 2 (LCDR2) defined by SEQ ID NO: 9, and a light chain complementary-determining region 3 (LCDR3) defined by SEQ ID NO: 10; and a heavy chain complementary-determining region 1 (HCDR1) defined by SEQ ID NO: 5, a heavy chain complementary-determining region 2 (HCDR2) defined by SEQ ID NO: 6, and a heavy chain complementary-determining region 3 (HCDR3) defined by SEQ ID NO: 7.

In various aspects and embodiments described herein, the anti-OSMRB antibody comprises a light chain variable domain having an amino acid sequence at least 90% identical to SEQ ID NO: 4; and a heavy chain variable domain having an amino acid sequence at least 90% identical to SEQ ID NO: 3. In some embodiments described herein, the light chain variable domain has the amino acid sequence set forth in SEQ ID NO: 4; and the heavy chain variable domain has the amino acid sequence set forth in SEQ ID NO: 3.

In various aspects and embodiments described herein, the anti-OSMRB antibody comprises CH1, hinge and CH2 domains derived from an IgG4 antibody fused to a CH3 domain derived from an IgG1 antibody.

In various aspects and embodiments described herein, the anti-OSMRB antibody comprises a light chain having an amino acid sequence at least 90% identical to SEQ ID NO: 2; and a heavy chain having an amino acid sequence at least 90% identical to SEQ ID NO: 1. In some embodiments described herein, the light chain has the amino acid sequence set forth in SEQ ID NO: 2; and the heavy chain has the amino acid sequence set forth in SEQ ID NO: 1.

In various aspects and embodiments described herein, the invention provided herein allows for treating a pruritic or inflammatory skin disease or disorder by using a therapeutically effective dose of anti-OSMRB antibody. In some embodiments, the therapeutically effective dose is equal to or greater than about 0.1 mg/kg, 0.2 mg/kg, 0.3 mg/kg, 0.4 mg/kg, 0.5 mg/kg, 0.6 mg/kg, 0.8 mg/kg, 0.9 mg/kg, 1 mg/kg, 1.2 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 17 mg/kg, 18 mg/kg, 19 mg/kg or 20 mg/kg. In some embodiments, the therapeutically effective dose is about between about 20 mg/kg and about 50 mg/kg. In some embodiments, the therapeutically effective dose is about 50 mg/kg and about 75 mg/kg. In some embodiments, the therapeutically effective dose is about between 75-100 mg/kg. In some embodiments, the therapeutically effective dose is about between 100 mg/kg and 125 mg/kg. In some embodiments, the therapeutically effective dose is about between 125 mg/kg and about 150 mg/kg. In some embodiments, the therapeutically effective dose is about between 175 mg/kg and 200 mg/kg.

In some embodiments, the therapeutically effective dose is approximately 3-20 mg/kg, approximately 4-20 mg/kg, approximately 5-20 mg/kg, approximately 6-20 mg/kg, approximately 7-20 mg/kg, approximately 8-20 mg/kg, approximately 9-20 mg/kg, approximately 10-20 mg/kg, approximately 11-20 mg/kg, approximately 12-20 mg/kg, approximately 13-20 mg/kg, approximately 14-20 mg/kg, approximately 15-20 mg/kg, approximately 16-20 mg/kg, approximately 17-20 mg/kg, approximately 18-20 mg/kg, approximately 19-20 mg/kg, approximately 3-19 mg/kg, approximately 3-18 mg/kg, approximately 3-17 mg/kg, approximately 3-16 mg/kg, approximately 3-15 mg/kg, approximately 3-14 mg/kg, approximately 3-13 mg/kg, approximately 3-12 mg/kg, approximately 3-11 mg/kg, approximately 3-10 mg/kg, approximately 3-9 mg/kg, approximately 3-8 mg/kg, approximately 3-7 mg/kg, approximately 3-6 mg/kg, approximately 3-5 mg/kg, approximately 3-4 mg/kg, or approximately 5-10 mg/kg. In some embodiments, a therapeutically effective dose is about 5 mg/kg. In some embodiments, a therapeutically effective dose is about 10 mg/kg.

In some embodiments, the therapeutically effective dose is equal to or greater than 20 mg/kg, 25 mg/kg, 30 mg/kg, 35 mg/kg, 40 mg/kg, 45 mg/kg, or 50 mg/kg.

In some embodiments, the therapeutically effective dose is equal to or greater than 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg.

In some embodiments, the therapeutically effective dose is approximately 50-1,000 mg/kg, approximately 100-1,000 mg/kg, approximately 150-1,000 mg/kg, approximately 200-1,000 mg/kg, approximately 250-1,000 mg/kg, approximately 300-1,000 mg/kg, approximately 350-1,000 mg/kg, approximately 400-1,000 mg/kg, approximately 450-1,000 mg/kg, approximately 500-1,000 mg/kg, approximately 550-1,000 mg/kg, approximately 600-1,000 mg/kg, approximately 650-1,000 mg/kg, approximately 700-1,000 mg/kg, approximately 750-1,000 mg/kg, approximately 800-1,000 mg/kg, approximately 850-1,000 mg/kg, approximately 900-1,000 mg/kg, approximately 950-1,000 mg/kg, approximately 50-950 mg/kg, approximately 50-900 mg/kg, approximately 50-850 mg/kg, approximately 50-800 mg/kg, approximately 50-750 mg/kg, approximately 50-700 mg/kg, approximately 50-650 mg/kg, approximately 50-600 mg/kg, approximately 50-550 mg/kg, approximately 50-500 mg/kg, approximately 50-450 mg/kg, approximately 50-400 mg/kg, approximately 50-350 mg/kg, approximately 50-300 mg/kg, approximately 50-250 mg/kg, approximately 50-200 mg/kg, approximately 50-150 mg/kg, or approximately 50-100 mg/kg.

In some embodiments, a therapeutically effective dose (e.g., an initial dose and/or a maintenance dose) is a flat dose. As used herein, the terms “flat dose” and “fixed dose” are used inter-changeably. In some embodiments, a suitable flat dose is between about 10 mg and 800 mg. Accordingly, in some embodiments, a suitable flat dose is equal to or greater than about 10 mg, 20 mg, 30 mg, 40 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385, 390 mg, 395 mg, 400 mg, 405 mg, 410 mg, 415, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 or 800 mg. In some embodiments, a suitable flat dose ranges from 50-800 mg, 50-700 mg, 50-600 mg, 50-500 mg, 100-800 mg, 100-700 mg, 100-600 mg, 100-500 mg, 100-500 mg, 100-400 mg, 150-400 mg, 200-400 mg, 250-400 mg, 300-350 mg, 320-400 mg, or 350-400 mg. In some embodiments, a suitable initial bolus or loading flat dose is about 720 mg. In some embodiments, a suitable maintenance flat dose is about 360 mg. In some embodiments, the flat dose is about 720 mg initial bolus or loading dose, and is about 360 mg maintenance dose.

In some embodiments, a loading dose is about 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In some embodiments, a maintenance dose is administered after administration of the loading dose. In some embodiments, a loading dose is between about 5 mg/kg and 25 mg/kg and a maintenance dose is between about 2.5 mg/kg and 7.5 mg/kg. In some embodiments, the maintenance dose is about 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.0 mg/kg, 6.5 mg/kg, 7.0 mg/kg, or 7.5 mg/kg. In some embodiments, a loading dose is about 10 mg/kg and maintenance dose is about 5 mg/kg.

In various aspects and embodiments described herein, administering the anti-OSMRβ antibody to a subject who has a pruritic or inflammatory skin disease or disorder results in a decrease in Numerical Rating Score (NRS) compared to a control.

In various aspects and embodiments described herein, the control is a NRS indicative of a subject with comparable disease status without treatment. In some embodiments, the control is a NRS in the subject prior to the treatment. In some embodiments, a control is the respective value of a disease parameter of a subject with comparable disease status but treated with a placebo.

In various aspects and embodiments described herein, NRS is decreased by at least 2-points, or by at least 3-points, or by at least 4-points, or by at least 5-points, or by at least 6 points, or by at least 7 points, or by at least 8 points. In some embodiments, the NRS is decreased by greater than 4-points. In some embodiments, the NRS is deceased by at least 8 points. In some embodiments, the NRS is decreased by approximately 10% or more, approximately 20% or more, approximately 30% or more, approximately 40% or more, approximately 50% or more, approximately 60% or more, approximately 70% or more, approximately 75% or more, or approximately 80% or more. In some embodiments, the decrease in NRS is approximately 4 or more points in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is approximately 5 points in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is 6 points or more in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is approximately 7 points or more in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is approximately 8 points or more in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is approximately 9 points or more in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS is approximately 10 points or more in approximately 30% or more, approximately 40% or more, approximately 50% or more, or approximately 60% or more, approximately 70% or more, or approximately 80% or more, of the subjects administered the anti-OSMRβ antibody. In some embodiments, the decrease in NRS occurs less than 5 weeks, or less than 4 weeks, or less than 3 weeks, or less than 2 weeks, or less than 1 week after the subject's initial dose of the anti-OSMRβ antibody. In some embodiments, the decrease in NRS is approximately 30% or more, approximately 40% or more, approximately 50% or more, or approximately 60% or more, approximately 70% or more, or approximately 80% or more, about 4 weeks after the subject's initial dose of the anti-OSMRβ antibody.

In various aspects and embodiments described herein, wherein the NRS is worst itch NRS (WI-NRS).

In various aspects and embodiments described herein, the NRS value is calculated as a weekly average.

In various aspects and embodiments described herein, administering the anti-OSMRβ antibody results in improved sleep in a subject as evidenced by a decrease in sleep-loss VAS from a compared to a control.

In various aspects and embodiments described herein, the control is a sleep-loss VAS indicative of a subject with comparable disease status without treatment. In some embodiments, the control is a sleep-loss VAS in the subject prior to the treatment. In some embodiments, the baseline is a sleep-loss VAS in the subject prior to the treatment.

In various aspects and embodiments described herein, the decrease in the sleep-loss VAS from the baseline is by at least 10%, or by at least 20%, or by at least 30%, or by at least 40%, or by at least 50%, or by at least 60%, or by at least 70%, or by at least 80%, or by at least 90%. In some embodiments, the decrease in the sleep-loss VAS occurs less than 5 weeks, or less than 4 weeks, or less than 3 weeks, or less than 2 weeks, or less than 1 week after the subject's initial dose of the anti-OSMR-β antibody.

In various aspects and embodiments described herein, the sleep-loss VAS value is calculated as a weekly average.

In various aspects and embodiments described herein, administering the anti-OSMRβ antibody results in a decrease in EASI compared to a control. In some embodiments, the control is an EASI indicative of a subject with comparable disease status without treatment. In some embodiments, the control is an EASI in the subject prior to the treatment. In some embodiments, the control is an EASI in the subject prior to the treatment. In some embodiments, the decrease in EASI from the baseline is by at least 10%, or by at least 20%, or by at least 30%, or by at least 40%, or by at least 50%, or by at least 60%, or by at least 70%, or by at least 80%, or by at least 90%. In some embodiments, the decrease in EASI occurs less than 5 weeks, or less than 4 weeks, or less than 3 weeks after the subject's initial dose of the anti-OSMRβ antibody.

In various aspects and embodiments described herein, the EASI value is calculated as a weekly average.

In various aspects and embodiments described herein, administering the anti-OSMRβ antibody results in two or more of: a decrease in pruritus Numerical Rating Score (NRS) by at least 4-points compared to a control NRS; a decrease in EASI by at least 20% compared to a control EASI; a decrease in sleep-loss VAS by at least 20% compared to a control VAS; an improvement in Scoring of Active Dermatitis (SCORAD) compared to a control SCORAD; an improvement in Dermatology Life Quality Index (DLQI) compared to a control DLQI; and an improvement in Hospital Anxiety and Depression Scale (HADS) compared to a control HADS. In some embodiments, administering the anti-OSMRβ antibody results in three or more, four or more, five or more, or six or more of the above identified decreases and improvements.

In various aspects and embodiments described herein, administering the anti-OSMRβ antibody results in a decrease in pruritus Numerical Rating Score (NRS) by at least 4-points compared to a control NRS, and a decrease in EASI by at least 20% compared to a control EASI.

In various aspects and embodiments described herein, administering the anti-OSMRβ antibody results in a decrease in pruritus Numerical Rating Score (NRS) by at least 4-points compared to a control NRS, and a decrease in sleep-loss VAS by at least 20% compared to a control VAS.

In various aspects and embodiments described herein, administering the anti-OSMRβ antibody results in a decrease in sleep-loss VAS by at least 20% compared to a control VAS, and a decrease in EASI by at least 20% compared to a control EASI.

In various aspects and embodiments described herein, administering the anti-OSMRβ antibody results in a decrease in pruritus Numerical Rating Score (NRS) by at least 4-points, 5-points, 6-points, 7-points, 8-points, or 9-points compared to the control NRS.

In various aspects and embodiments described herein, administering the anti-OSMRβ antibody results in a decrease in EASI by at least 30%, or by at least 40%, or by at least 50%, or by at least 60%, or by at least 70%, by at least 75%, or by at least 80%, or by at least 90% compared to the control EASI. In some embodiments, administering the anti-OSMRβ antibody to subjects results in a decrease in EASI score by 50% (i.e., EASI-50) in approximately 30% or more of the subjects, in approximately 35% or more of the subjects, approximately 40% or more of the subjects, in approximately 45% or more of the subjects, approximately 50% or more of the subjects, in approximately 55% or more of the subjects, approximately 60% or more of the subjects, in approximately 65% or more of the subjects, approximately 70% or more of the subjects, in approximately 75% or more of the subjects, in approximately 80% or more of the subjects, or approximately 85% or more of the subjects. In various aspects and embodiments described herein, administering the anti-OSMRβ antibody to subjects results in a decrease in EASI score by 75% (i.e., EASI-75) in approximately 30% or more of the subjects, in approximately 35% or more of the subjects, approximately 40% or more of the subjects, in approximately 45% or more of the subjects, approximately 50% or more of the subjects, in approximately 55% or more of the subjects, approximately 60% or more of the subjects, in approximately 65% or more of the subjects, approximately 70% or more of the subjects, in approximately 75% or more of the subjects, in approximately 80% or more of the subjects, or approximately 85% or more of the subjects.

In various aspects and embodiments described herein, administering the anti-OSMRβ antibody results in a decrease in sleep-loss VAS by at least 30%, or by at least 40%, or by at least 50%, or by at least 60%, or by at least 70%, or by at least 80%, or by at least 90% compared to the control VAS.

In various aspects and embodiments described herein, the control is a value indicative of a respective parameter (e.g., NRS, EASI, VAS, SCORAD, DLQI, or HADS) in a subject with comparable disease status without treatment. In various aspects and embodiments described herein, the control is a value indicative of a respective parameter (e.g., NRS, EASI, VAS, SCORAD, DLQI, or HADS) in a subject prior to the treatment. In various aspects and embodiments described herein, the control is a value indicative of a respective parameter (e.g., NRS, EASI, VAS, SCORAD, DLQI, or HADS) in a subject with comparable disease status but treated with a placebo.

In various aspects and embodiments described herein, the invention provides a method of treating inflammation, the method comprising administering to a subject in need of treatment an anti-OSMRβ antibody at a therapeutically effective dose and an administration interval for a treatment period such that one or more symptoms associated with inflammation are reduced in intensity, severity, or frequency or has delayed in onset. In some embodiments, the inflammation is T_{H}2 mediated inflammation. In some embodiments, the inflammation is independent of IL-31.

In various aspects and embodiments described herein, the subject is suffering from an inflammatory disease, disorder or condition. In some embodiments, the subject is suffering from a chronic inflammatory disease. In some embodiments, the chronic inflammatory disease is Chronic Idiopathic Urticaria (CIU) and the symptom associated with inflammation that is reduced in intensity, severity, or frequency or has delayed in onset is urticaria.

In various aspects and embodiments described herein, an anti-OSMRβ antibody described herein is administered in conjunction with an additional therapeutic agent. In some embodiments, the additional therapeutic agent is a topical corticosteroid (e.g., TCS). In some embodiments, the additional therapeutic agent is a topical calcineurin inhibitor. In some embodiments, the additional therapeutic agent is a topical antimicrobial and/or antiseptic. In some embodiments, the additional therapeutic agent is a topical antihistamine.

It is to be understood that all embodiments as described above are applicable to all aspects of the present invention.

### BRIEF DESCRIPTION OF THE DRAWING

The drawings are for illustration purposes only not for limitation.

FIG. 1A depicts an exemplary graph of the percent inhibition of scratching behavior in monkeys 1 hour after IL-31 challenge at 2, 8, 15, 21 and 29 days after dosing with an anti-OSMRβ antibody.

FIG. 1B depicts graphs of scratching behavior and serum concentration of an anti-OSMRβ antibody in monkeys 1 hour after IL-31 challenge at 2, 8, 15, 21 and 29 days after dosing with the anti-OSMRβ antibody.

FIG. 2 depicts study design for determining safety and efficacy of the anti-OSMRβ antibody in a single dose dose-escalation study in healthy volunteers and atopic dermatitis patients.

FIG. 3A-3D depicts change in pruritus with anti-OSMRβ antibody treatment. Patients received single intravenous dose of 7.5 mg/kg anti-OSMRβ antibody (anti-OSMRβ Ab) or placebo (PBO). FIG. 3A indicates change in mean VAS pruritus score (+/−SEM) from baseline over the indicated period. FIG. 3B indicates the mean percent VAS pruritus score change from baseline (+/−SEM) over the indicated period. FIG. 3C indicates mean weekly average worst itch NRS (WI-NRS) over the indicated period. FIG. 3D indicates the mean percent change in weekly average WI-NRS from baseline (+/−SEM). The data indicate greater reduction in patients receiving the antibody as compared to PBO within the first 4 weeks after administration, which persisted up to 8 weeks.

FIG. 4 depicts percentage of subjects having clinically meaningful reduction in weekly average NRS (≥4 points) after receiving single intravenous dose of 7.5 mg/kg anti-OSMRβ antibody (anti-OSMRβ Ab) or placebo (PBO).

FIGS. 5A-5D depicts the number of subjects who responded with a particular magnitude of NRS reduction from baseline (≥4) over 9 weeks after single intravenous dose of 7.5 mg/kg anti-OSMRβ antibody (anti-OSMRβ Ab) or placebo. FIGS. 5A and 5C show results from the anti-OSMRβ antibody recipient group, and FIGS. 5B and 5D show the results from the placebo group.

FIG. 6A-6B shows improvement from sleeplessness over the course of the indicated observation period in subjects receiving single intravenous dose of 7.5 mg/kg anti-OSMRβ antibody (anti-OSMRβ Ab) or placebo. FIG. 6A shows mean (±SEM) sleep-loss VAS score change, FIG. 6B shows Mean (±SEM) Percent sleep-loss VAS score change from baseline.

FIGS. 7A-7B depicts the changes in EASI score as a measure of the disease severity. Subjects received single intravenous dose of 7.5 mg/kg anti-OSMRβ antibody (anti-OSMRβ Ab) or placebo. FIG. 7A shows the mean scores (±SEM); FIG. 7B shows mean percent (±SEM) EASI change from baseline.

FIGS. 8A-8B depict percent of subjects showing of clinically meaningful response of having a reduction in disease severity as measured by EASI score. Subjects received single intravenous dose of 7.5 mg/kg anti-OSMRβ antibody (anti-OSMRβ Ab) or placebo. FIG. 8A shows results for responders having a EASI reduction of 50% or more compared to baseline (EASI-50 score); FIG. 8B shows results for responders having a EASI reduction of 75% or more compared to baseline (EASI-75) respectively, over the study period after single intravenous dose of 7.5 mg/kg anti-OSMRβ antibody (anti-OSMRβ Ab) or placebo. % indicated above each data point indicates the percent of subjects in the population of the group. Empty bars denote placebo subjects, solid bars denote anti-OSMRβ Ab recipient subjects in FIGS. 8A and 8B.

FIG. 9A-9B shows SCORAD values as an over clinical evaluation of change in the extent and severity of atopic dermatitis after subjects received single intravenous dose of 7.5 mg/kg anti-OSMRβ antibody (anti-OSMRβ Ab) or placebo. FIG. 9A shows mean SCORAD values (+/−SEM) change from baseline over the indicated period. FIG. 9B shows mean percent SCORAD (+/−SEM) change from baseline over the indicated period.

FIG. 10A-10C depicts modeled PK parameters for subcutaneous administration. FIG. 10A depicts simulated median values of anti-OSMR Ab concentration in plasma in various dosing regimens indicated in the inset. FIG. 10B depicts concentration profiles of anti-OSMRβ Ab in plasma over indicated time period after subcutaneous administered to atopic dermatitis patients. HV, healthy volunteers; AD, atopic dermatitis patients; IV, intravenous administration; SC, subcutaneous administration. FIG. 10C depicts a range of simulations for various SC dosing regimens.

FIG. 11 is a schematic that depicts IL-31Rα, OSMRβ and LIFR signaling pathways.

FIG. 12 is a series of graphs that depict MCP-1 protein levels in the supernatants of human epidermal keratinocytes (HEK) and human dermal fibroblasts (HDF) following treatment with OSM (50 ng/mL) for 6 hours and 24 hours (panel A). FIG. 12, panel B shows MCP-1 mRNA levels relative to the housekeeping gene 18S mRNA. The data show strong upregulation of MCP-1 levels following addition of OSM.

FIG. 13 is a series of graphs that depict MCP-1 protein levels in the supernatants of cultured HEK and HDF cells following addition of 50 ng/mL OSM, 50 ng/mL LIF, or 100 ng/mL of IL-31 in combination with increasing concentrations of IL-4 (panel A) or IL-13 (panel B).

FIG. 14 is a series of graphs that shows the mRNA expression levels of IL-13Rα1 or IL-4Rα in cultured HEK cells treated with OSM for 6 hours and 24 hours.

FIG. 15 is a series of graphs that show the effect of adding either anti-OSMRβ antibody (panel A), anti-IL-31Rα antibody (panel B) or an isotype control (panel C) at increasing concentrations to cultured HEK cells that had been treated with OSM at 50 ng/mL.

FIG. 16 is a series of graphs that show the effect of adding either anti-OSMRβ antibody (panel A), anti-IL-31Rα antibody (panel B) or an isotype control (panel C) at increasing concentrations to cultured HEK cells that had been stimulated with OSM at 50 ng/mL and IL-4 (at either 5 or 20 ng/mL concentrations).

FIG. 17 is a series of graphs that depict the results of IL-31 mRNA expression measurements obtained from non-lesional (NL) and lesional (LS) skin biopsies of subjects who have prurigo nodularis (PN) or atopic dermatitis (AD).

FIG. 18 is a series of graphs that depict the results of IL-31 mRNA expression measurements (panel A) or OSM expression measurements (panel B) obtained from PN, AD or from healthy control subject (HC) skin biopsies.

FIG. 19 is a series of graphs that show the results of OSM (panel A) and IL-31 (panel B) mRNA expression measurements obtained from PN subjects who either had WI-NRS<7 or who had WI-NRS≥7.

FIG. 20 is a series of graphs that show quantitation of immunohistochemistry observations in skin samples obtained from PN subjects. FIG. 20, panels A-D show quantitation of cells (cells/μm2) found in the dermis that are positive for OSMRβ (panel A), OSM (panel B), IL-31 (panel C), or IL-31Rα (panel D) in samples obtained from PN subjects in comparison to healthy controls. FIG. 20, panels E-H are graphs that show percent positivity for IL-31 (panel E), OSM (panel (F), IL-31α (panel G), or OSMRβ (panel H) in skin samples obtained from NL or LS skin biopsies of PN subjects.

FIG. 21 is a series of graphs that show quantitation of immunohistochemistry observations (IL-31, panel A; OSM, panel B; IL-31Rα, panel C; OSMRβ, panel D) obtained from NL skin biopsies, and from LS skin biopsies from subjects who had either WI-NRS<7 or WI-NRS≥7.

FIG. 22 is a series of graphs that show OSMRβ mRNA (panels A and B) or protein (panel C) expression levels obtained from control skin samples or skin samples obtained from chronic idiopathic urticaria patients. Panels A and B show OSMRβ mRNA expression levels as detected using RNAscope® or NanoString® technologies, respectively. Panel C shows OSMRβ protein expression levels as determined by immunohistochemistry.

FIG. 23 is a series of graphs that show OSMRβ mRNA levels in subjects who have Lichen Simplex Chronicus (LSC). OSMRβ mRNA levels in samples obtained from LSC patients was assessed by NanoString (panel A) and RNAscope (panel B) technology.

FIG. 24 is a graph that shows OSMRβ mRNA levels in subjects who have Lichen Planus (LP). OSMRβ mRNA levels in samples obtained from LP patients was assessed using NanoString technology.

FIG. 25 is a graph that shows OSMRβ mRNA levels in subjects who have Chronic Idiopathic Pruritus (C1P). OSMRβ mRNA levels in samples obtained from C1P patients was assessed using NanoString technology.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxyl- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes increasing levels of relevant protein or its activity that is deficient in relevant disease tissues.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Control: As used herein, the term control is a reference based on which a change is determined. In some embodiments, a control is the respective value of a disease parameter of a subject with comparable disease status, but without treatment. In some embodiments, a control is the respective value of a disease parameter of a subject with comparable disease status but treated with a placebo. In some embodiments, a control is the respective value of a disease parameter of a subject prior to treatment (also referred to as baseline). In some embodiments, a control is a reference value indicative of a disease parameter without treatment based on collective knowledge, or historical data.

Delivery: As used herein, the term “delivery” encompasses both local and systemic delivery.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as the respective value of a disease parameter of a subject with comparable disease status, but without a treatment described herein, or a measurement in a subject (or multiple control subjects) in the absence of the treatment described herein, e.g., a subject who is administered a placebo. In some embodiments, a control is a reference value indicative of a disease parameter without treatment, based on collective knowledge, or historical data.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J Mal. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suitable for subcutaneous delivery: As used herein, the phrase “suitable for subcutaneous delivery” or “formulation for subcutaneous delivery” as it relates to the pharmaceutical compositions of the present invention generally refers to the stability, viscosity, tolerability and solubility properties of such compositions, as well as the ability of such compositions to deliver an effective amount of antibody contained therein to the targeted site of delivery.

Patient: As used herein, the term “patient” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms. A “patient” is used interchangeably with “subject” where the subject has a disease and is administered either the antibody or a placebo.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Target tissues: As used herein, the term “target tissues” refers to any tissue that is affected by a disease or disorder to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, methods of treating atopic dermatitis comprising a step of administering to a subject in need of treatment an anti-OSMRβ antibody at a therapeutically effective dose and an administration interval for a treatment period sufficient to improve, stabilize or reduce one or more symptoms of atopic dermatitis relative to a control. Also provided are methods of treating uremic pruritus, comprising a step of administering to a subject in need of treatment an anti-OSMRβ antibody at a therapeutically effective dose and an administration interval for a treatment period sufficient to improve, stabilize or reduce one or more symptoms of atopic dermatitis relative to a control.

Various aspects of the invention are described in detail in the following sections. The use of sections is not meant to limit the invention. Each section can apply to any aspect of the invention. In this application, the use of “or” means “and/or” unless stated otherwise.

### Atopic Dermatitis

Atopic dermatitis (AD) is a chronic inflammatory skin disease, characterized by Th2 cell-mediated immune responses, impaired skin barrier function, and bacterial colonization. The prevalence of AD is about 20% in children and 1% to 10% in adults. Approximately 20% of patients with AD have moderate to severe disease involving large body surface areas and suffer from chronic intense pruritus, leading to sleep deprivation and poor quality of life (Boguniewicz et al., 2011; Brandt et al., 2011; Gittler et al., 2012; Silverberg et al., 2013). Topical corticosteroid and calcineurin inhibitors are used for the treatment of moderate to severe disease, but these therapies have limited efficacy, and prolonged use is associated with side effects. Similarly, systemic corticosteroids or cyclosporine, though efficacious, are associated with significant toxicities (Ring et al., 2012; Sidbury et al., 2014).

In pruritic conditions, the IL-31 axis has been consistently shown to be up-regulated. Serum levels of IL-31 were elevated and correlated with AD disease severity in children (Ezzat et al., 2011) and in adults (Raap et al., 2008). Increased IL-31 mRNA was observed in skin biopsies from AD and PN patients compared to healthy skin (Sonkoly et al., 2006); and IL-31, OSMRB, and IL-31 receptor a (IL-31Rα) staining was enhanced in AD skin (Nobbe et al., 2012). IL-31 is produced by activated Th2 cells (Dillon et al., 2004), and its expression is induced by IL-4 (Stott et al., 2013). Accordingly, peripheral blood mononuclear cells (PBMC) from atopic donors produce more IL-31 upon activation compared with PBMCs from non-atopic donors (Stott et al., 2013). Once released, IL-31 participates in a feedback loop that perpetuates the inflammatory response in AD. IL-31 increases the production of IL-4, IL-5, and IL-13 in PBMCs from atopic donors and in nasal epithelial cells (Liu et al., 2015). In addition, IL-31 synergizes with IL-4 in production of CCL2, VEGF, and, very importantly, in the induction of more IL-4, IL-5, and IL-13 (Ip et al., 2007; Stott et al., 2013; Liu et al., 2015).

Another exacerbating factor comes from the role of colonizing bacteria, such as Staphylococcus, that occasionally infect the skin in AD. Staphylococcal Enterotoxin B (SEB) and Staphylococcal a toxin, super antigens produced by Staphylococcus, increase the production of IL-31 in PBMCs and skin of AD patients (Sonkoly et al., 2006; Niebuhr et al., 2011), further reinforcing the vicious cycle of inflammation. The inflammatory response is also reinforced on the cytokine receptor side. Keratinocytes and skin-infiltrating macrophages in AD express IL-31Rα; and SEB, TLR2 agonists (a cellular component of Staphylococcus), IFN-γ, OSM, IL-4, and IL-13 upregulate the expression of IL-31Rα on macrophages and keratinocytes (Bilsborough et al., 2006; Heise et al., 2009; Kasraie et al., 2011; Edukulla et al., 2015). Clinical data on the importance of IL-31 in AD symptomatology and disease progression are provided by the first clinical trial with CIM331, a humanized anti-IL31Rα antibody also known as nemolizumab, in which AD patients showed reduced pruritus visual analogue scale (VAS) scores (Nemoto et al., 2016; Ruzicka et al., 2017).

OSM also plays an important role in AD pathology and echoes many of the functions of IL-31. OSM is produced by skin infiltrating T cells in AD, and OSMRβ levels are increased in the skin of AD patients (Boniface et al., 2007). In addition to skin-infiltrating T cells, OSM is produced by macrophages and neutrophils under inflammatory conditions (Richards, 2013). Once produced, OSM induces the production of multiple cytokines: IL-4, IL-5, IL-13, IL-6, IL-12, tumor necrosis factor (TNF), and IL-10, and chemokines (CXCL1, CXCL2, CXCL8, CCL11, and CCL24) (Fritz et al., 2011; Botelho et al., 2013). In addition, it promotes collagen deposition through a mechanism independent of transforming growth factor-β, IL-4/IL-13, lymphocytes, and mast cells (Mozaffarian et al., 2008). In the inflammatory milieu, OSM synergizes with IL-4 to produce eotaxin, an eosinophil chemoattractant (Fritz et al., 2006; Fritz et al., 2009). Furthermore, OSM synergizes with IL-1, TNF, IL-17, and IL-22 to down-regulate genes involved in keratinocyte differentiation and skin barrier integrity (desmoglein and filaggrin), and to upregulate human-beta-defensin (HBD) 2 and HBD3 (Boniface et al., 2007; Rabeony et al., 2014). HBD2 and HBD3, in turn, feed into the vicious cycle of inflammation by inducing the production of more OSM, IL-22, IL-4, IL-13, and IL-31 (Kanda et al., 2012). This cycle is further fueled by OSM upregulation of IL-4Rα (Mozaffarian et al., 2008; Fritz et al., 2009; Fritz et al., 2011). Collectively, IL-31 and OSM reinforce the inflammatory response and compromise the skin barrier function in AD through multiple overlapping pathways. Thus, an antibody, such as the anti-OSMRβ antibodies described herein, that antagonizes both IL-31 and OSM provides a therapeutic opportunity in AD through the inhibition of downstream signaling events stimulated by IL-31 and OSM, two cytokines that drive pruritus and inflammation.

There are several different methods for assessing symptoms of atopic dermatitis. In some embodiments, one or more symptoms of atopic dermatitis are assessed by an Investigators' Global Assessment (IGA) of atopic dermatitis. In some embodiments, one or more symptoms of atopic dermatitis are assessed by an Eczema Area and Severity Index (EASI). In some embodiments, one or more symptoms of atopic dermatitis are assessed by scoring atopic dermatitis (SCORAD). In some embodiments, one or more symptoms of atopic dermatitis are assessed by atopic dermatitis Area Photographs. In some embodiments, one or more symptoms of atopic dermatitis are assessed by Body Surface Area Involvement (BSA) of Atopic Dermatitis. In some embodiments, one or more symptoms of atopic dermatitis are assessed by a Dermatology Life Quality Index (DLQI). In some embodiments, one or more symptoms of atopic dermatitis are assessed by a Hospital Anxiety and Depression Scale (HADS). In some embodiments, one or more symptoms of atopic dermatitis, such as sleep quality and sleep quantity, are assessed by actigraphy. In some embodiments, one or more symptoms of atopic dermatitis are assessed by a quantitative numerical pruritus scale, e.g., Pruritus Numerical Rating Scale (NRS), Visual Analogue Scale (VAS) or Verbal Rating Scale (VRS).

Treatment

In some embodiments of the invention, atopic dermatitis is treated by administering to a subject in need of treatment an anti-OSMRβ antibody at a therapeutically effective dose and an administration interval for a treatment period sufficient to improve, stabilize or reduce one or more symptoms of atopic dermatitis relative to a control. The terms, “treat” or “treatment,” as used in the context of atopic dermatitis herein, refers to amelioration of one or more symptoms associated with atopic dermatitis, prevention or delay of the onset of one or more symptoms of atopic dermatitis, and/or lessening of the severity or frequency of one or more symptoms of atopic dermatitis. In some embodiments, the terms, “treat” or “treatment,” as used in the context of atopic dermatitis herein, refers to partially or completely alleviate, ameliorate, relieve, inhibit, prevent, delay onset of, reduce severity of and/or reduce incidence of one or more symptoms or features of atopic dermatitis. In some embodiments, the administration of an anti-OSMRβ antibody results in a statistically-significant drop on a quantitative numerical pruritus scale. In some embodiments, the step of administering comprises subcutaneous administration. In some embodiments, subcutaneous administration is through subcutaneous injection. In some embodiments, subcutaneous administration is through a subcutaneous pump.

In some embodiments, subcutaneous injection of the anti-OSMRβ antibody can be performed in the upper arm, the anterior surface of the thigh, the lower portion of the abdomen, the upper back or the upper area of the buttock. In some embodiments, the site of injection is rotated. In some embodiments, the step of administering comprises intravenous administration. In some embodiments, the step of administering comprises intravenous administration followed by subcutaneous administration.

In some embodiments, the effect of an anti-OSMRβ antibody on atopic dermatitis is measured relative to a control. In some embodiments, a control is indicative of the one or more symptoms of atopic dermatitis in the subject before the treatment. In some embodiments, one or more symptoms of atopic dermatitis in a subject before treatment comprises a score on a pruritus NRS greater than or equal to 5. In some embodiments, one or more symptoms of atopic dermatitis in a subject before treatment comprises a score on a pruritus NRS greater than or equal to 7. In some embodiments, a subject in need of treatment has been diagnosed with atopic dermatitis for at least one year. In some embodiments, a subject in need of treatment has been diagnosed with moderate to severe atopic dermatitis. In some embodiments, moderate to severe atopic dermatitis comprises an IGA score of 3 or 4. In some embodiments, moderate to severe atopic dermatitis comprises a BSA involvement of approximately 10% or more. In some embodiments, moderate to severe atopic dermatitis comprises an IGA score of 3 or 4 and BSA involvement of approximately 10% or more. In some embodiments, a control is indicative of the one or more symptoms of atopic dermatitis in a control subject with the same disease status without treatment. In some embodiments, the control is indicative of the one or more symptoms of atopic dermatitis in a control subject with the same disease status that was administered a placebo.

In some embodiments, a subject in need of treatment has elevated levels of one or more cytokines associated with the OSMRβ signaling pathway in comparison to a healthy subject. Accordingly, in some embodiments, the subject in need of treatment has elevated levels of one or more of IL-31, OSM, IL-31Rα, and OSMRβ in comparison to a healthy subject. In some embodiments, the subject in need of treatment has elevated levels of one or more of IL-31 in comparison to a healthy subject. In some embodiments, the subject in need of treatment has elevated levels of one or more of OSM in comparison to a healthy subject. In some embodiments, the subject in need of treatment has elevated levels of one or more of IL-31Rα in comparison to a healthy subject. In some embodiments, the subject in need of treatment has elevated levels of one or more of OSMRβ in comparison to a healthy subject.

In some embodiments, treating the subject in need thereof results in a decrease or stabilization of MCP-1/CCL2 levels in the subject. Accordingly, in some embodiments, treating a subject in need thereof results in a decrease of MCP-1 levels in comparison to the diseased state. In some embodiments, treating a subject in need thereof results in stabilization of MCP-1 levels. By “stabilization” is meant that the levels of MCP-1 remain about the same and do not increase or decrease. In some embodiments, treating a subject results in reduced MCP-1 levels in lymphocytes and/or endothelial cells.

In some embodiments, the subject in need of treatment has WI-NRS scores of about 4, about 5, about 6, about 7, about 8 or above. Accordingly, in some embodiments, the subject in need of treatment has WI-NRS score of about 4. In some embodiments, the subject in need of treatment has WI-NRS score of about 5. In some embodiments, the subject in need of treatment has WI-NRS score of about 6. In some embodiments, the subject in need of treatment has WI-NRS score of about 7. In some embodiments, the subject in need of treatment has WI-NRS score of about 8. In some embodiments, the subject in need of treatment has WI-NRS score of more than 8.

In some embodiments, a subject is selected for treatment who has MCP-1/CCL2 levels greater than found in a healthy individual. In some embodiments, the subject selected for treatment does not have elevated levels of MCP-1/CCL2 in comparison to a healthy individual. In some embodiments, IL-31 expression level is elevated in the subject relative to a control. In some embodiments, IL-31 expression level is not elevated in the subject relative to a control. In some embodiments, IL-31 expression level in a portion of the subject's skin affected by a pruritic disease or condition is approximately the same as the IL-31 expression level in (i) a portion of the subject's skin that is unaffected by the pruritic disease or condition, or (ii) a portion of normal skin from a healthy subject, who is not diagnosed with a pruritic disease or condition. In some embodiments, IL-31Rα expression level is elevated in the subject relative to a control. In some embodiments, OSM expression level is elevated in the subject relative to a control. In some embodiments, OSMRβ expression level is elevated in the subject relative to a control. In some embodiments, OSMRβ expression level is not elevated in the subject relative to a control. In some embodiments, OSMRβ expression level in a portion of the subject's skin affected by a pruritic disease or condition is approximately the same as the OSMRβ expression level in (i) a portion of the subject's skin that is unaffected by the pruritic disease or condition, or (ii) a portion of normal skin from a healthy subject, who is not diagnosed with a pruritic disease or condition.

Dosage

A therapeutically effective dose of an anti-OSMRβ antibody for treating atopic dermatitis can occur at various dosages. In some embodiments of the invention, a therapeutically effective dose is equal to or greater than about 0.1 mg/kg, 0.2 mg/kg, 0.3 mg/kg, 0.4 mg/kg, 0.5 mg/kg, 0.6 mg/kg, 0.8 mg/kg, 0.9 mg/kg, 1 mg/kg, 1.2 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 17 mg/kg, 18 mg/kg, 19 mg/kg or 20 mg/kg, or 30 mg/kg. In some embodiments, a therapeutically effective dose is equal to or greater than 3 mg/kg, 4 mg/kg, 5 mg/kg, 6 mg/kg, 7 mg/kg, 7.5 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg.

In some embodiments a therapeutically effective dose is approximately 0.1-20 mg/kg, approximately 0.3-20 mg/kg, approximately 0.5-20 mg/kg, approximately 0.75-20 mg/kg, approximately 1-20 mg/kg, approximately 1.5-20 mg/kg, approximately 2-20 mg/kg, approximately 2.5-20 mg/kg, approximately 3-20 mg/kg, approximately 3.5-20 mg/kg, approximately 4-20 mg/kg, approximately 4.5-20 mg/kg, approximately 5-20 mg/kg, approximately 5.5-20 mg/kg, approximately 6-20 mg/kg, approximately 6.5-20 mg/kg, approximately 7-20 mg/kg, approximately 7.5-20 mg/kg, approximately 8-20 mg/kg, approximately 8.5-20 mg/kg, approximately 9-20 mg/kg, approximately 9.5-20 mg/kg, approximately 10-20 mg/kg, approximately 10.5-20 mg/kg.

In some embodiments, a therapeutically effective dose is approximately 3-20 mg/kg, approximately 4-20 mg/kg, approximately 5-20 mg/kg, approximately 6-20 mg/kg, approximately 7-20 mg/kg, approximately 8-20 mg/kg, approximately 9-20 mg/kg, approximately 10-20 mg/kg, approximately 11-20 mg/kg, approximately 12-20 mg/kg, approximately 13-20 mg/kg, approximately 14-20 mg/kg, approximately 15-20 mg/kg, approximately 16-20 mg/kg, approximately 17-20 mg/kg, approximately 18-20 mg/kg, approximately 19-20 mg/kg, approximately 3-19 mg/kg, approximately 3-18 mg/kg, approximately 3-17 mg/kg, approximately 3-16 mg/kg, approximately 3-15 mg/kg, approximately 3-14 mg/kg, approximately 3-13 mg/kg, approximately 3-12 mg/kg, approximately 3-11 mg/kg, approximately 3-10 mg/kg, approximately 3-9 mg/kg, approximately 3-8 mg/kg, approximately 3-7 mg/kg, approximately 3-6 mg/kg, approximately 3-5 mg/kg, or approximately 3-4 mg/kg, or approximately 5-10 mg/kg. In some embodiments, a therapeutically effective dose is about 5 mg/kg. In some embodiments, a therapeutically effective dose is about 10 mg/kg.

In some embodiments, the therapeutically effective dose is equal to or greater than 50 mg/kg, 100 mg/kg, 150 mg/kg, 200 mg/kg, or 250 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 450 mg/kg, 500 mg/kg, 550 mg/kg, 600 mg/kg, 650 mg/kg, 700 mg/kg, 710 mg/kg, 720 mg/kg, 730 mg/kg, 740 mg/kg, 750 mg/kg, 800 mg/kg, 850 mg/kg, 900 mg/kg, 950 mg/kg, or 1000 mg/kg.

In some embodiments, a therapeutically effective dose is approximately 50-1,000 mg/kg, approximately 100-1,000 mg/kg, approximately 150-1,000 mg/kg, approximately 200-1,000 mg/kg, approximately 250-1,000 mg/kg, approximately 300-1,000 mg/kg, approximately 350-1,000 mg/kg, approximately 400-1,000 mg/kg, approximately 450-1,000 mg/kg, approximately 500-1,000 mg/kg, approximately 550-1,000 mg/kg, approximately 600-1,000 mg/kg, approximately 650-1,000 mg/kg, approximately 700-1,000 mg/kg, approximately 750-1,000 mg/kg, approximately 800-1,000 mg/kg, approximately 850-1,000 mg/kg, approximately 900-1,000 mg/kg, approximately 950-1,000 mg/kg, approximately 50-950 mg/kg, approximately 50-900 mg/kg, approximately 50-850 mg/kg, approximately 50-800 mg/kg, approximately 50-750 mg/kg, approximately 50-700 mg/kg, approximately 50-650 mg/kg, approximately 50-600 mg/kg, approximately 50-550 mg/kg, approximately 50-500 mg/kg, approximately 50-450 mg/kg, approximately 50-400 mg/kg, approximately 50-350 mg/kg, approximately 50-300 mg/kg, approximately 50-250 mg/kg, approximately 50-200 mg/kg, approximately 50-150 mg/kg, or approximately 50-100 mg/kg.

In some embodiments, administering comprises an initial bolus or loading dose, followed by at least one maintenance dose. In some embodiments, the initial bolus or loading dose is greater than the at least one maintenance dose. In some embodiments, the initial bolus or loading dose is at least one-fold, two-fold, three-fold, four fold or five-fold greater in dosage than the dosage of the at least one maintenance dose. In some embodiments, the initial bolus or loading dose is two-fold greater in dosage than the dosage of the at least one maintenance dose. For example, in some embodiments, the initial bolus or loading dose is 720 mg and the maintenance dose is 360 mg.

In some embodiments, a maintenance dose is administered after administration of the loading dose. In some embodiments, a flat dose is used as an initial bolus or loading dose and/or maintenance dose. In some embodiments, a suitable flat dose is provided in a single injection syringe. A suitable flat dose may be administered (e.g., subcutaneously or intravenously) in a single injection or by multiple injections. In some embodiments, a suitable flat dose is about between 10 mg and 800 mg. Accordingly, in some embodiments, a suitable flat dose is equal to or greater than about 10 mg, 20 mg, 30 mg, 40 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385, 390 mg, 395 mg, 400 mg, 405 mg, 410 mg, 415, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 or 800 mg. In some embodiments, a suitable flat dose ranges from 50-800 mg, 50-700 mg, 50-600 mg, 50-500 mg, 100-800 mg, 100-700 mg, 100-600 mg, 100-500 mg, 100-500 mg, 100-400 mg, 150-400 mg, 200-400 mg, 250-400 mg, 300-350 mg, 320-400 mg, or 350-400 mg. In some embodiments, a loading dose is about 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800. In some embodiments, a suitable initial bolus flat dose is 720 mg. In some embodiments, a maintenance dose is about 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 380 mg, 390 mg, 395 mg, or 400 mg. In some embodiments, a suitable maintenance flat dose is 360 mg. In some embodiments, the flat dose is 720 mg initial bolus dose, and is 360 mg maintenance dose. In some embodiments an initial loading or bolus dose of about 720 mg is administered. In some embodiments, the therapeutically effective dose comprises an initial bolus or loading dose of about 720 mg, followed by at least one maintenance dose of about 360 mg.

In some embodiments, a weight-based dose is used as an initial bolus or loading dose and/or maintenance dose. In some embodiments, the dose is provided in a single injection syringe. The dose may be administered (e.g., subcutaneously or intravenously) in a single injection or by multiple injections. In some embodiments, a loading dose is about 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In some embodiments, a loading dose is about between 5 mg/kg and 25 mg/kg and a maintenance dose is about between 2.5 mg/kg and 7.5 mg/kg. In some embodiments, the maintenance dose is about 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.0 mg/kg, 6.5 mg/kg, 7.0 mg/kg, or 7.5 mg/kg. In some embodiments an initial loading or bolus dose of about 10 mg/kg is administered. In some embodiments, the therapeutically effective dose comprises an initial bolus dose of about 10 mg/kg, followed by at least one maintenance dose of about 5 mg/kg.

Administration Interval

An administration interval of an anti-OSMRβ antibody in the treatment of atopic dermatitis can occur at various durations. In some embodiments of the invention, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

Treatment Period

A treatment period of atopic dermatitis with an anti-OSMRβ antibody can vary in duration. In some embodiments, the treatment period is at least one month. In some embodiments the treatment period is at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 13 weeks, or at least 15 weeks, or at least 18 weeks, or at least 20 weeks, or at least 22 weeks, or at least 24 weeks. In some embodiments, the treatment period is at least two months. In some embodiments, the treatment period is at least three months. In some embodiments, the treatment period is at least six months. In some embodiments, the treatment period is at least nine months. In some embodiments, the treatment period is at least one year. In some embodiments, the treatment period is at least two years. In some embodiments, the treatment period continues throughout the subject's life.

Pharmacokinetics and Pharmacodynamics

Evaluation of anti-OSMRβ antibody concentration-time profiles in serum of subjects with atopic dermatitis may be evaluated directly by measuring systemic serum anti-OSMRβ antibody concentration-time profiles. Typically, anti-OSMRβ antibody pharmacokinetic and pharmacodynamic profiles are evaluated by sampling the blood of treated subjects periodically. The following standard abbreviations are used to represent the associated pharmacokinetic parameters.
Cₘₐₓ maximum concentration
tₘₐₓ time to maximum concentration
AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λz is the apparent terminal elimination rate constant
λz apparent terminal elimination rate constant, where λz is the magnitude of the slope of the linear regression of the log concentration versus time profile during the terminal phase
t_{1/2} apparent terminal elimination half-life (whenever possible), where t_{1/2}=natural log (ln)(2)/λz
CL clearance
Vd volume of distribution (IV doses only)
Vd/F apparent volume of distribution (SC doses only)

Typically, actual blood sample collection times relative to the start of anti-OSMRβ antibody administration are used in PK analysis. For example, blood samples are typically collected within 15 or 30 minutes prior to anti-OSMRβ antibody administration (pre-injection baseline or time 0) and at hours 1, 4, 8 or 12, or days 1 (24 hours), 2, 3, 4, 5, 6, 7, 10, 14, 17, 21, 24, 28, 31, 38, 45, 52, 60, 70 or 90 days, following administration.

Various methods may be used to measure anti-OSMRβ antibody concentration in serum. As a non-limiting example, enzyme-linked immunosorbent assay (ELISA) methods are used.

Pharmacokinetic parameters may be evaluated at any stage during the treatment, for example, at day 1, day 2, day 3, day 4, day 5, day 6, week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or later. In some embodiments, pharmacokinetic parameters may be evaluated at month 1, month 2, month 3, month 4, month 5, month 6, month 7, month 8, month 9, month 10, month 11, month 12, month 13, month 14, month 15, month 16, month 17, month 18, month 19, month 20, month 21, month 22, month 23, month 24, or later during the treatment.

Adverse Effects

Adverse effects related to the treatment of atopic dermatitis can include peripheral edema, exacerbation of atopic dermatitis, nasopharyngitis, upper respiratory tract infections, increased creatine phosphokinase, conjunctivitis, blepharitis, oral herpes, keratitis, eye pruritus, other herpes simplex virus infection, and dry eye.

In some embodiments, administration of an anti-OSMRβ antibody results in no serious adverse effects in the subject. In some embodiments, administration of an anti-OSMRβ antibody does not result in one or more of peripheral edema, exacerbation of atopic dermatitis, nasopharyngitis, upper respiratory tract infections, increased creatine phosphokinase, conjunctivitis, blepharitis, oral herpes, keratitis, eye pruritus, other herpes simplex virus infection, and dry eye.

Combination Therapy

In some embodiments, an anti-OSMRβ antibody described herein may be used in combination with one or more other therapeutic agents for the treatment of atopic dermatitis (AD). For example, an anti-OSMRβ antibody may be administered in combination with one or more of concomitant corticosteroids (e.g., TCS), topical calcineurin inhibitors, antimicrobials and/or antiseptics, antihistamines, and others (e.g., coal tar, phosphodiesterase inhibitors) that are administered systemically (e.g., orally) or topically. In some embodiments, an anti-OSMRβ antibody and one or more other therapeutic agents may be administered simultaneously. In some embodiments, an anti-OSMRβ antibody and one or more other therapeutic agents may be administered sequentially. In some embodiments, one or more other therapeutic agents may be administered as needed.

### Uremic Pruritus

Uremic pruritus (UP) is a debilitating disease with a significant negative impact on patient quality of life. Roughly more than half of patients with end stage renal disease (ESRD) undergoing dialysis suffer from pruritus (Makhlough, 2010). The prevalence of moderate to severe disease has been estimated at 42% based on results from an international dialysis outcomes and practice study (Pisoni et al., 2006). The underlying etiology of UP is unknown, but IL-31 has been implicated. In fact, in UP, a minimum threshold concentration was identified for circulating IL-31 above which pruritus dramatically increased, suggesting serum IL-31 may be a quantitative biomarker of pruritus intensity, at least in that indication (Ko et al., 2014). Patients are often treated with moisturizers, topical steroids, antihistamines, phototherapy (ultraviolet B light), cholestyramine, erythropoietin, and ondansetron, but efficacy is poor with these therapies, and thus novel therapeutic approaches are urgently needed (Makhlough et al, 2010). Thus, an antibody, such as the anti-OSMRβ antibodies described herein, that antagonizes both IL-31 and OSM, provides a therapeutic opportunity in UP through the inhibition of downstream signaling events stimulated by IL-31 and OSM, two cytokines that drive pruritus, inflammation, and fibrosis in chronic pruritic diseases.

The clinical characteristics of uremic pruritus are variable. Pruritus may be constant or intermittent. The back is the most commonly affected area, but arms, head, and abdomen are also commonly affected. Excoriations with no primary lesions, and sparing of the butterfly area of the back, are typical. Patients with ESRD, especially if attributable to diabetes mellitus, frequently develop keratotic nodules that on biopsy show a perforating disorder. These represent prurigo nodules and are a marker for severe and long-term pruritus.

There are several different methods for assessing symptoms of uremic pruritus. In some embodiments, one or more symptoms of uremic pruritus are assessed by a Pruritus Numerical Rating Scale (NRS). In some embodiments, one or more symptoms of uremic pruritus are assessed by a Dermatology Life Quality Index (DLQI). In some embodiments, one or more symptoms of uremic pruritus are assessed by a Hospital Anxiety and Depression Scale (HADS). In some embodiments, one or more symptoms of uremic pruritus, such as sleep quality and sleep quantity, are assessed by actigraphy.

In some embodiments, the methods of the invention are used for treating pruritus in a subject having a kidney disease. In some embodiments, the methods of the invention are used for treating pruritus in subjects having chronic kidney disease. In some embodiments, the methods of the invention are used in predialysis subjects having chronic kidney disease. The composition and the methods of the invention are useful in the treating pruritus in a subgroup of subjects having chronic kidney disease, and who have not undergone dialysis. In some embodiments, administering of an anti-OSMRβ antibody occurs prior to, during, or immediately following dialysis.

Treatment

In some embodiments of the invention, uremic pruritus is treated by administering to a subject in need of treatment an anti-OSMRβ antibody at a therapeutically effective dose and an administration interval for a treatment period sufficient to improve, stabilize or reduce one or more symptoms of uremic pruritus relative to a control. The terms, “treat” or “treatment,” as used in the context of uremic pruritus herein, refers to amelioration of one or more symptoms associated with uremic pruritus, prevention or delay of the onset of one or more symptoms of uremic pruritus, and/or lessening of the severity or frequency of one or more symptoms of uremic pruritus. In some embodiments, the terms, “treat” or “treatment,” as used in the context of uremic pruritus herein, refers to partially or completely alleviate, ameliorate, relieve, inhibit, prevent, delay onset of, reduce severity of and/or reduce incidence of one or more symptoms or features of uremic pruritus. In some embodiments, the administration of an anti-OSMRβ antibody results in a statistically-significant drop on a quantitative numerical pruritus scale. In some embodiments, the step of administering comprises subcutaneous administration. In some embodiments, subcutaneous administration is through subcutaneous injection. In some embodiments, subcutaneous administration is through a subcutaneous pump.

In some embodiments, subcutaneous injection of the anti-OSMRβ antibody can be performed in the upper arm, the anterior surface of the thigh, the lower portion of the abdomen, the upper back or the upper area of the buttock. In some embodiments, the site of injection is rotated.

In some embodiments, the step of administering comprises intravenous administration. In some embodiments, the step of administering comprises intravenous administration followed by subcutaneous administration. In some embodiments, the step of administering occurs one day before the subject undergoes hemodialysis. In other embodiments, the step of administering occurs during hemodialysis. In other embodiments, the step of administering occurs within one day after hemodialysis.

In some embodiments, the subject in need of treatment has end stage renal disease. In some embodiments, the subject in need of treatment is undergoing a hemodialysis regimen of at least one time-per-week. In some embodiments, the subject in need of treatment is undergoing a three-times-per-week hemodialysis regimen. In some embodiments, the three-times-per-week hemodialysis regimen has been stable for at least three months.

In some embodiments, the effect of an anti-OSMRβ antibody on uremic pruritus is measured relative to a control. In some embodiments, a control is indicative of the one or more symptoms of uremic pruritus in the subject before the treatment. In some embodiments, one or more symptoms of uremic pruritus in a subject before treatment comprises a score on a pruritus NRS greater than or equal to 5. In some embodiments, one or more symptoms of uremic pruritus in a subject before treatment comprises a score on a pruritus NRS greater than or equal to 7. In some embodiments, a control is indicative of the one or more symptoms of uremic pruritus in a control subject with the same disease status without treatment. In some embodiments, the control is indicative of the one or more symptoms of uremic pruritus in a control subject with the same disease status that was administered a placebo.

In some embodiments, a subject in need of treatment of an inflammatory or pruritic skin disease or disorder in accordance with the invention has elevated levels of one or more cytokines associated with the OSMRβ signaling pathway in comparison to a healthy subject. Accordingly, in some embodiments, the subject in need of treatment has elevated levels of one or more of IL-31, OSM, IL-31Rα, and OSMRβ in comparison to a healthy subject. In some embodiments, the subject in need of treatment has elevated levels of one or more of IL-31 in comparison to a healthy subject. In some embodiments, the subject in need of treatment has elevated levels of one or more of OSM in comparison to a healthy subject. In some embodiments, the subject in need of treatment has elevated levels of one or more of IL-31Rα in comparison to a healthy subject. In some embodiments, the subject in need of treatment has elevated levels of one or more of OSMRβ in comparison to a healthy subject.

In some embodiments, treating the subject in need thereof results in a decrease or stabilization of MCP-1/CCL2 levels in the subject. Accordingly, in some embodiments, treating a subject in need thereof results in a decrease of MCP-1 levels in comparison to the diseased state. In some embodiments, treating a subject in need thereof results in stabilization of MCP-1 levels. By “stabilization” is meant that the levels of MCP-1 remain about the same and do not increase or decrease. In some embodiments, treating a subject results in reduced MCP-1 levels in lymphocytes and/or endothelial cells.

In some embodiments, the subject in need of treatment has WI-NRS scores of about 4, about 5, about 6, about 7, about 8 or above. Accordingly, in some embodiments, the subject in need of treatment has WI-NRS score of about 4. In some embodiments, the subject in need of treatment has WI-NRS score of about 5. In some embodiments, the subject in need of treatment has WI-NRS score of about 6. In some embodiments, the subject in need of treatment has WI-NRS score of about 7. In some embodiments, the subject in need of treatment has WI-NRS score of about 8. In some embodiments, the subject in need of treatment has WI-NRS score of more than 8.

In some embodiments, a subject is selected for treatment who has MCP-1/CCL2 levels greater than found in a healthy individual. In some embodiments, the subject selected for treatment does not have elevated levels of MCP-1/CCL2 in comparison to a healthy individual. In some embodiments, IL-31 expression level is elevated in the subject relative to a control. In some embodiments, IL-31 expression level is not elevated in the subject relative to a control. In some embodiments, IL-31 expression level in a portion of the subject's skin affected by a pruritic disease or condition is approximately the same as the IL-31 expression level in (i) a portion of the subject's skin that is unaffected by the pruritic disease or condition, or (ii) a portion of normal skin from a healthy subject, who is not diagnosed with a pruritic disease or condition. In some embodiments, IL-31Rα expression level is elevated in the subject relative to a control. In some embodiments, OSM expression level is elevated in the subject relative to a control. In some embodiments, OSMRβ expression level is elevated in the subject relative to a control. In some embodiments, OSMRβ expression level is not elevated in the subject relative to a control. In some embodiments, OSMRβ expression level in a portion of the subject's skin affected by a pruritic disease or condition is approximately the same as the OSMRβ expression level in (i) a portion of the subject's skin that is unaffected by the pruritic disease or condition, or (ii) a portion of normal skin from a healthy subject, who is not diagnosed with a pruritic disease or condition.

Dosage

A therapeutically effective dose of an anti-OSMRβ antibody for treating uremic pruritus or for treating pruritus in predialysis subjects having kidney disease can occur at various dosages. In some embodiments of the invention, a therapeutically effective dose is equal to or greater than about 0.1 mg/kg, 0.2 mg/kg, 0.3 mg/kg, 0.4 mg/kg, 0.5 mg/kg, 0.6 mg/kg, 0.8 mg/kg, 0.9 mg/kg, 1 mg/kg, 1.2 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 17 mg/kg, 18 mg/kg, 19 mg/kg or 20 mg/kg, or 30 mg/kg. In some embodiments, a therapeutically effective dose is equal to or greater than 3 mg/kg, 4 mg/kg, 5 mg/kg, 6 mg/kg, 7 mg/kg, 7.5 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg.

In some embodiments a therapeutically effective dose is approximately 0.1-20 mg/kg, approximately 0.3-20 mg/kg, approximately 0.5-20 mg/kg, approximately 0.75-20 mg/kg, approximately 1-20 mg/kg, approximately 1.5-20 mg/kg, approximately 2-20 mg/kg, approximately 2.5-20 mg/kg, approximately 3-20 mg/kg, approximately 3.5-20 mg/kg, approximately 4-20 mg/kg, approximately 4.5-20 mg/kg, approximately 5-20 mg/kg, approximately 5.5-20 mg/kg, approximately 6-20 mg/kg, approximately 6.5-20 mg/kg, approximately 7-20 mg/kg, approximately 7.5-20 mg/kg, approximately 8-20 mg/kg, approximately 8.5-20 mg/kg, approximately 9-20 mg/kg, approximately 9.5-20 mg/kg, approximately 10-20 mg/kg, approximately 10.5-20 mg/kg.

In some embodiments, a therapeutically effective dose is approximately 3-20 mg/kg, approximately 4-20 mg/kg, approximately 5-20 mg/kg, approximately 6-20 mg/kg, approximately 7-20 mg/kg, approximately 8-20 mg/kg, approximately 9-20 mg/kg, approximately 10-20 mg/kg, approximately 11-20 mg/kg, approximately 12-20 mg/kg, approximately 13-20 mg/kg, approximately 14-20 mg/kg, approximately 15-20 mg/kg, approximately 16-20 mg/kg, approximately 17-20 mg/kg, approximately 18-20 mg/kg, approximately 19-20 mg/kg, approximately 3-19 mg/kg, approximately 3-18 mg/kg, approximately 3-17 mg/kg, approximately 3-16 mg/kg, approximately 3-15 mg/kg, approximately 3-14 mg/kg, approximately 3-13 mg/kg, approximately 3-12 mg/kg, approximately 3-11 mg/kg, approximately 3-10 mg/kg, approximately 3-9 mg/kg, approximately 3-8 mg/kg, approximately 3-7 mg/kg, approximately 3-6 mg/kg, approximately 3-5 mg/kg, or approximately 3-4 mg/kg, or approximately 5-10 mg/kg. In some embodiments, a therapeutically effective dose is about 5 mg/kg. In some embodiments, a therapeutically effective dose is about 10 mg/kg.

In some embodiments, the therapeutically effective dose is equal to or greater than 50 mg/kg, 100 mg/kg, 150 mg/kg, 200 mg/kg, or 250 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 450 mg/kg, 500 mg/kg, 550 mg/kg, 600 mg/kg, 650 mg/kg, 700 mg/kg, 710 mg/kg, 720 mg/kg, 730 mg/kg, 740 mg/kg, 750 mg/kg, 800 mg/kg, 850 mg/kg, 900 mg/kg, 950 mg/kg, or 1000 mg/kg.

In some embodiments, a therapeutically effective dose is approximately 50-1,000 mg/kg, approximately 100-1,000 mg/kg, approximately 150-1,000 mg/kg, approximately 200-1,000 mg/kg, approximately 250-1,000 mg/kg, approximately 300-1,000 mg/kg, approximately 350-1,000 mg/kg, approximately 400-1,000 mg/kg, approximately 450-1,000 mg/kg, approximately 500-1,000 mg/kg, approximately 550-1,000 mg/kg, approximately 600-1,000 mg/kg, approximately 650-1,000 mg/kg, approximately 700-1,000 mg/kg, approximately 750-1,000 mg/kg, approximately 800-1,000 mg/kg, approximately 850-1,000 mg/kg, approximately 900-1,000 mg/kg, approximately 950-1,000 mg/kg, approximately 50-950 mg/kg, approximately 50-900 mg/kg, approximately 50-850 mg/kg, approximately 50-800 mg/kg, approximately 50-750 mg/kg, approximately 50-700 mg/kg, approximately 50-650 mg/kg, approximately 50-600 mg/kg, approximately 50-550 mg/kg, approximately 50-500 mg/kg, approximately 50-450 mg/kg, approximately 50-400 mg/kg, approximately 50-350 mg/kg, approximately 50-300 mg/kg, approximately 50-250 mg/kg, approximately 50-200 mg/kg, approximately 50-150 mg/kg, or approximately 50-100 mg/kg.

In some embodiments, administering comprises an initial bolus or loading dose, followed by at least one maintenance dose. In some embodiments, the initial bolus or loading dose is greater than the at least one maintenance dose. In some embodiments, the initial bolus or loading dose is at least one-fold, two-fold, three-fold, four fold or five-fold greater in dosage than the dosage of the at least one maintenance dose. In some embodiments, the initial bolus or loading dose is two-fold greater in dosage than the dosage of the at least one maintenance dose. For example, in some embodiments, the initial bolus or loading dose is 720 mg and the maintenance dose is 360 mg.

In some embodiments, a maintenance dose is administered after administration of the loading dose. In some embodiments, a flat dose is used as an initial bolus or loading dose and/or maintenance dose. In some embodiments, a suitable flat dose is provided in a single injection syringe. A suitable flat dose may be administered (e.g., subcutaneously or intravenously) in a single injection or by multiple injections. In some embodiments, a suitable flat dose is about between 10 mg and 800 mg. Accordingly, in some embodiments, a suitable flat dose is equal to or greater than about 10 mg, 20 mg, 30 mg, 40 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385, 390 mg, 395 mg, 400 mg, 405 mg, 410 mg, 415, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 or 800 mg. In some embodiments, a suitable flat dose ranges from 50-800 mg, 50-700 mg, 50-600 mg, 50-500 mg, 100-800 mg, 100-700 mg, 100-600 mg, 100-500 mg, 100-500 mg, 100-400 mg, 150-400 mg, 200-400 mg, 250-400 mg, 300-350 mg, 320-400 mg, or 350-400 mg. In some embodiments, a loading dose is about 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800. In some embodiments, a suitable initial bolus flat dose is 720 mg. In some embodiments, a maintenance dose is about 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 380 mg, 390 mg, 395 mg, or 400 mg. In some embodiments, a suitable maintenance flat dose is 360 mg. In some embodiments, the flat dose is 720 mg initial bolus dose, and is 360 mg maintenance dose. In some embodiments an initial loading or bolus dose of about 720 mg is administered. In some embodiments, the therapeutically effective dose comprises an initial bolus or loading dose of about 720 mg, followed by at least one maintenance dose of about 360 mg.

In some embodiments, a weight-based dose is used as an initial bolus or loading dose and/or maintenance dose. In some embodiments, the dose is provided in a single injection syringe. The dose may be administered (e.g., subcutaneously or intravenously) in a single injection or by multiple injections. In some embodiments, a loading dose is about 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In some embodiments, a loading dose is about between 5 mg/kg and 25 mg/kg and a maintenance dose is about between 2.5 mg/kg and 7.5 mg/kg. In some embodiments, the maintenance dose is about 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.0 mg/kg, 6.5 mg/kg, 7.0 mg/kg, or 7.5 mg/kg. In some embodiments an initial loading or bolus dose of about 10 mg/kg is administered. In some embodiments, the therapeutically effective dose comprises an initial bolus dose of about 10 mg/kg, followed by at least one maintenance dose of about 5 mg/kg.

Administration Interval

An administration interval of an anti-OSMRβ antibody in the treatment of uremic pruritus or treatment of pruritus in a chronic kidney disease subject can occur at various durations. In some embodiments of the invention, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

Treatment Period

A treatment period of uremic pruritus with an anti-OSMRβ antibody can vary in duration. In some embodiments, the treatment period is at least one month. In some embodiments the treatment period is at least 4 weeks, or at least 5 weeks, or at least 6 weeks, or at least 7 weeks, or at least 8 weeks, or at least 9 weeks, or at least 10 weeks, or at least 11 weeks or at least 12 weeks, or at least 13 weeks, or at least 15 weeks, or at least 18 weeks, or at least 20 weeks, or at least 22 weeks, or at least 24 weeks. In some embodiments, the treatment period is at least two months. In some embodiments, the treatment period is at least three months. In some embodiments, the treatment period is at least six months. In some embodiments, the treatment period is at least nine months. In some embodiments, the treatment period is at least one year. In some embodiments, the treatment period is at least two years. In some embodiments, the treatment period is for as long as the subject is on hemodialysis.

Pharmacokinetics and Pharmacodynamics

Evaluation of anti-OSMRβ antibody concentration-time profiles in serum of subjects with uremic pruritus may be evaluated directly by measuring systemic serum anti-OSMRβ antibody concentration-time profiles. Typically, anti-OSMRβ antibody pharmacokinetic and pharmacodynamic profiles are evaluated by sampling the blood of treated subjects periodically. The following standard abbreviations are used to represent the associated pharmacokinetic parameters.
Cₘₐₓ maximum concentration
tₘₐₓ time to maximum concentration
AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λz is the apparent terminal elimination rate constant
λz apparent terminal elimination rate constant, where λz is the magnitude of the slope of the linear regression of the log concentration versus time profile during the terminal phase
t_{1/2} apparent terminal elimination half-life (whenever possible), where t_{1/2}=natural log (ln)(2)/λz
CL clearance
Vd volume of distribution (IV doses only)
Vd/F apparent volume of distribution (SC doses only)

Typically, actual blood sample collection times relative to the start of anti-OSMRβ antibody administration are used in PK analysis. For example, blood samples are typically collected within 15 or 30 minutes prior to anti-OSMRβ antibody administration (pre-injection baseline or time 0) and at hours 1, 4, 8 or 12, or days 1 (24 and 28 hours), 2, 3, 4, 5, 6, 7, 10, 13, 17, 20, 24, 27, 31, 34, 41, 48, 55, 62, 69, 76, 90, following administration.

Various methods may be used to measure anti-OSMRβ antibody concentration in serum. As a non-limiting example, enzyme-linked immunosorbent assay (ELISA) methods are used.

Pharmacokinetic parameters may be evaluated at any stage during the treatment, for example, at day 1, day 2, day 3, day 4, day 5, day 6, week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or later. In some embodiments, pharmacokinetic parameters may be evaluated at month 1, month 2, month 3, month 4, month 5, month 6, month 7, month 8, month 9, month 10, month 11, month 12, month 13, month 14, month 15, month 16, month 17, month 18, month 19, month 20, month 21, month 22, month 23, month 24, or later during the treatment.

Adverse Effects

Adverse effects related to the treatment of uremic pruritus can include peripheral edema, nasopharyngitis, upper respiratory tract infections, increased creatine phosphokinase, conjunctivitis, blepharitis, oral herpes, keratitis, eye pruritus, other herpes simplex virus infection, and dry eye.

In some embodiments, administration of an anti-OSMRβ antibody results in no serious adverse effects in the subject. In some embodiments, administration of an anti-OSMRβ antibody does not result in one or more of peripheral edema, nasopharyngitis, upper respiratory tract infections, and increased creatine phosphokinase.

Combination Therapy

In some embodiments, an anti-OSMRβ antibody described herein may be used in combination with one or more other therapeutic agents for the treatment of uremic pruritus (UP). For example, an anti-OSMRβ antibody may be administered in combination with one or more of concomitant corticosteroids (e.g., TCS), calcineurin inhibitors, antimicrobials and/or antiseptics, antihistamines, and others (e.g., coal tar, phosphodiesterase inhibitors) that are administered systemically (e.g., orally) or topically. In some embodiments, an anti-OSMRβ antibody and one or more other therapeutic agents may be administered simultaneously. In some embodiments, an anti-OSMRβ antibody and one or more other therapeutic agents may be administered sequentially. In some embodiments, one or more other therapeutic agents may be administered as needed.

### Prurigo Nodularis

In one aspect, an anti-OSMRβ antibody described herein is used in treating prurigo nodularis (PN). In some embodiments, the methods of the invention are used for treating pruritus in a subject having PN. PN, also known as nodular prurigo is a skin disease characterized by itchy nodules. The nodules usually appear in the arms and legs. Patients often present with multiple excoriating lesions caused by scratching. In some embodiments, the subject presents with pruritic hyperkeratotic nodules.

In some embodiments, the prurigo nodularis is idiopathic. In some embodiments, the prurigo nodularis is not associated with any other underlying co-morbidities. In some embodiments, the prurigo nodularis is associated with one or more underlying co-morbidities.

In some embodiments, PN can be a distinct, highly pruritic chronic skin disease that is not defined by its comorbid conditions. IL-31 could be implicated in the pathogenesis of PN. In some embodiments, the IL-31 pathway could be an attractive target for pharmacological intervention in PN. In some embodiments, IL-31 expression level is elevated in the subject relative to a control. In some embodiments, IL-31Rα expression level is elevated in the subject relative to a control. In some embodiments, OSM expression level is elevated in the subject relative to a control. In some embodiments, OSMR expression level is elevated in the subject relative to a control. In some embodiments, the levels of any one of IL-31, IL-31Rα, OSM and OSMR in the subject is determined via skin biopsy from hyperkeratotic nodules. In some embodiments, the control is a healthy subject, who is not diagnosed with a pruritic disease.

The method of treating prurigo nodularis comprises administering to the subject in need of treatment an anti-OSMRβ antibody at a therapeutically effective dose and an administration interval for a treatment period sufficient to improve, stabilize or reduce one or more symptoms of pruritus relative to a control.

In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection. There are several different methods for assessing symptoms of prurigo nodularis. In some embodiments, one or more symptoms of prurigo nodularis are assessed by a change or percent change from baseline in Pruritus Numerical Rating Scale (NRS). In some embodiments, one or more symptoms of prurigo nodularis are assessed by a change or percent change from baseline in weekly average of Worst Itch-Numeric Rating Scale (WI-NRS). In some embodiments, one or more symptoms of prurigo nodularis are assessed by the proportion of subjects achieving at least a 4-point reduction from baseline in weekly average WI-NRS. In some embodiments, one or more symptoms of prurigo nodularis are assessed by a change or percent change from baseline in pruritus Visual Analog Scale (VAS). In some embodiments, one or more symptoms of prurigo nodularis are assessed by change from baseline in 5-D Pruritus total score. In some embodiments, one or more symptoms of prurigo nodularis are assessed by change from baseline in Sleep Loss VAS. In some embodiments, one or more symptoms of prurigo nodularis are assessed by change from baseline in weekly average of difficulty falling asleep NRS. In some embodiments, one or more symptoms of prurigo nodularis are assessed by change from baseline in weekly average of sleep quality NRS. In some embodiments, one or more symptoms of prurigo nodularis are assessed by change from baseline in quality of life measures over time. In some embodiments, one or more symptoms of prurigo nodularis are assessed by change from baseline in Prurigo Nodularis Investigor Global Assessment (PN-IGA). In some embodiments, one or more symptoms of prurigo nodularis are assessed by change from baseline in Prurigo Nodularis Nodule Assessment Tool (PN-NAT). In some embodiments, one or more symptoms of prurigo nodularis are assessed by a Dermatology Life Quality Index (DLQI). In some embodiments, one or more symptoms of prurigo nodularis are assessed by a Hospital Anxiety and Depression Scale (HADS). In some embodiments, one or more symptoms of prurigo nodularis, such as sleep quality and sleep quantity, are assessed by actigraphy.

Treatment

In some embodiments of the invention, prurigo nodularis is treated by administering to a subject in need of treatment an anti-OSMRβ antibody at a therapeutically effective dose and an administration interval for a treatment period sufficient to improve, stabilize or reduce one or more symptoms of prurigo nodularis relative to a control. The terms, “treat” or “treatment,” as used in the context of prurigo nodularis herein, refers to amelioration of one or more symptoms associated with prurigo nodularis, prevention or delay of the onset of one or more symptoms of prurigo nodularis, and/or lessening of the severity or frequency of one or more symptoms of prurigo nodularis. In some embodiments, the terms, “treat” or “treatment,” as used in the context of prurigo nodularis herein, refers to partially or completely alleviate, ameliorate, relieve, inhibit, prevent, delay onset of, reduce severity of and/or reduce incidence of one or more symptoms or features of prurigo nodularis. In some embodiments, the administration of an anti-OSMRβ antibody results in a statistically-significant drop on a quantitative numerical pruritus scale. In some embodiments, the administration of an anti-OSMRβ antibody results in a statistically-significant drop in weekly average Worst Itch-Numerical Rating Scale (WI-NRS). In some embodiments, the weekly average WI-NRS score has at least a 4-point reduction from baseline. In some embodiments, the administration of an anti-OSMRβ antibody results in a statistically-significant drop or percent change from baseline in pruritus Visual Analog Scale (VAS). In some embodiments, the administration of an anti-OSMRβ antibody results in a statistically-significant drop or percent change from baseline in 5-D Pruritus total score. In some embodiments, the administration of an anti-OSMRβ antibody results in a statistically-significant drop or percent change from baseline in Sleep Loss VAS. In some embodiments, the administration of an anti-OSMRβ antibody results in a statistically-significant drop or percent change in weekly average of difficulty falling asleep NRS. In some embodiments, the administration of an anti-OSMRβ antibody results in a statistically-significant drop or percent change in weekly average sleep quality NRS. In some embodiments, the administration of an anti-OSMRβ antibody results in a statistically-significant drop or percent change from baseline in Prurigo Nodularis Investigator Global Assessment (PN-IGA). In some embodiments, the administration of an anti-OSMRβ antibody results in a statistically-significant drop or percent change from baseline in Prurigo Nodularis Nodule Assessment Tool (PN-NAT). In some embodiments, the administration of an anti-OSMRβ antibody results in a statistically-significant decrease or percent change from baseline in Dermatology Life Quality Index (DLQI). In some embodiments, the administration of an anti-OSMRβ antibody results in a statistically-significant decrease or percent change from baseline in Hospital Anxiety and Depression Scale (HADS). In some embodiments, the administration of an anti-OSMRβ antibody results in a statistically-improved or percent change from baseline in actigraphy scores. In some embodiments, the administration of an anti-OSMRβ antibody results in a statistically-significant increase or percent change in quality of life measures over time. In some embodiments, the administration of an anti-OSMRβ antibody results in a statistically-significant drop or percent change in UAS7 score. In some embodiments, the step of administering comprises subcutaneous administration. In some embodiments, subcutaneous administration is through subcutaneous injection. In some embodiments, subcutaneous administration is through a subcutaneous pump.

In some embodiments, subcutaneous injection of the anti-OSMRβ antibody can be performed in the upper arm, the anterior surface of the thigh, the lower portion of the abdomen, the upper back or the upper area of the buttock. In some embodiments, the site of injection is rotated.

In some embodiments, the step of administering comprises intravenous administration. In some embodiments, the step of administering comprises intravenous administration followed by subcutaneous administration. In some embodiments, the step of administering occurs one day before the subject undergoes hemodialysis. In other embodiments, the step of administering occurs during hemodialysis. In other embodiments, the step of administering occurs within one day after hemodialysis.

In some embodiments, the subject in need of treatment has end stage renal disease. In some embodiments, the subject in need of treatment is undergoing a hemodialysis regimen of at least one time-per-week. In some embodiments, the subject in need of treatment is undergoing a three-times-per-week hemodialysis regimen. In some embodiments, the three-times-per-week hemodialysis regimen has been stable for at least three months.

In some embodiments, the effect of an anti-OSMRβ antibody on prurigo nodularis is measured relative to a control. In some embodiments, a control is indicative of the one or more symptoms of prurigo nodularis in the subject before the treatment. In some embodiments, one or more symptoms of prurigo nodularis in a subject before treatment comprises a score on a pruritus NRS greater than or equal to 5. In some embodiments, one or more symptoms of prurigo nodularis in a subject before treatment comprises a score on a pruritus NRS greater than or equal to 7. In some embodiments, a control is indicative of the one or more symptoms of prurigo nodularis in a control subject with the same disease status without treatment. In some embodiments, the control is indicative of the one or more symptoms of prurigo nodularis in a control subject with the same disease status that was administered a placebo.

In some embodiments, a subject in need of treatment has elevated levels of one or more cytokines associated with the OSMRβ signaling pathway in comparison to a healthy subject. Accordingly, in some embodiments, the subject in need of treatment has elevated levels of one or more of IL-31, OSM, IL-31Rα, and OSMRβ in comparison to a healthy subject. In some embodiments, the subject in need of treatment has elevated levels of one or more of IL-31 in comparison to a healthy subject. In some embodiments, the subject in need of treatment has elevated levels of one or more of OSM in comparison to a healthy subject. In some embodiments, the subject in need of treatment has elevated levels of one or more of IL-31Rα in comparison to a healthy subject. In some embodiments, the subject in need of treatment has elevated levels of one or more of OSMRβ in comparison to a healthy subject.

In some embodiments, treating the subject in need thereof results in a decrease or stabilization of MCP-1/CCL2 levels in the subject. Accordingly, in some embodiments, treating a subject in need thereof results in a decrease of MCP-1 levels in comparison to the diseased state. In some embodiments, treating a subject in need thereof results in stabilization of MCP-1 levels. By “stabilization” is meant that the levels of MCP-1 remain about the same and do not increase or decrease. In some embodiments, treating a subject results in reduced MCP-1 levels in lymphocytes and/or endothelial cells.

In some embodiments, the subject in need of treatment has WI-NRS scores of about 4, about 5, about 6, about 7, about 8 or above. Accordingly, in some embodiments, the subject in need of treatment has WI-NRS score of about 4. In some embodiments, the subject in need of treatment has WI-NRS score of about 5. In some embodiments, the subject in need of treatment has WI-NRS score of about 6. In some embodiments, the subject in need of treatment has WI-NRS score of about 7. In some embodiments, the subject in need of treatment has WI-NRS score of about 8. In some embodiments, the subject in need of treatment has WI-NRS score of more than 8.

In some embodiments, a subject is selected for treatment who has MCP-1/CCL2 levels greater than found in a healthy individual. In some embodiments, the subject selected for treatment does not have elevated levels of MCP-1/CCL2 in comparison to a healthy individual. In some embodiments, IL-31 expression level is elevated in the subject relative to a control. In some embodiments, IL-31 expression level is not elevated in the subject relative to a control. In some embodiments, IL-31 expression level in a portion of the subject's skin affected by a pruritic disease or condition is approximately the same as the IL-31 expression level in (i) a portion of the subject's skin that is unaffected by the pruritic disease or condition, or (ii) a portion of normal skin from a healthy subject, who is not diagnosed with a pruritic disease or condition. In some embodiments, IL-31Rα expression level is elevated in the subject relative to a control. In some embodiments, OSM expression level is elevated in the subject relative to a control. In some embodiments, OSMRβ expression level is elevated in the subject relative to a control. In some embodiments, OSMRβ expression level is not elevated in the subject relative to a control. In some embodiments, OSMRβ expression level in a portion of the subject's skin affected by a pruritic disease or condition is approximately the same as the OSMRβ expression level in (i) a portion of the subject's skin that is unaffected by the pruritic disease or condition, or (ii) a portion of normal skin from a healthy subject, who is not diagnosed with a pruritic disease or condition.

Dosage

A therapeutically effective dose of an anti-OSMRβ antibody for treating prurigo nodularis can occur at various dosages. In some embodiments of the invention, a therapeutically effective dose is equal to or greater than about 0.1 mg/kg, 0.2 mg/kg, 0.3 mg/kg, 0.4 mg/kg, 0.5 mg/kg, 0.6 mg/kg, 0.8 mg/kg, 0.9 mg/kg, 1 mg/kg, 1.2 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 17 mg/kg, 18 mg/kg, 19 mg/kg or 20 mg/kg, or 30 mg/kg. In some embodiments, a therapeutically effective dose is equal to or greater than 3 mg/kg, 4 mg/kg, 5 mg/kg, 6 mg/kg, 7 mg/kg, 7.5 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg.

In some embodiments a therapeutically effective dose is approximately 0.1-20 mg/kg, approximately 0.3-20 mg/kg, approximately 0.5-20 mg/kg, approximately 0.75-20 mg/kg, approximately 1-20 mg/kg, approximately 1.5-20 mg/kg, approximately 2-20 mg/kg, approximately 2.5-20 mg/kg, approximately 3-20 mg/kg, approximately 3.5-20 mg/kg, approximately 4-20 mg/kg, approximately 4.5-20 mg/kg, approximately 5-20 mg/kg, approximately 5.5-20 mg/kg, approximately 6-20 mg/kg, approximately 6.5-20 mg/kg, approximately 7-20 mg/kg, approximately 7.5-20 mg/kg, approximately 8-20 mg/kg, approximately 8.5-20 mg/kg, approximately 9-20 mg/kg, approximately 9.5-20 mg/kg, approximately 10-20 mg/kg, approximately 10.5-20 mg/kg.

In some embodiments, a therapeutically effective dose is approximately 3-20 mg/kg, approximately 4-20 mg/kg, approximately 5-20 mg/kg, approximately 6-20 mg/kg, approximately 7-20 mg/kg, approximately 8-20 mg/kg, approximately 9-20 mg/kg, approximately 10-20 mg/kg, approximately 11-20 mg/kg, approximately 12-20 mg/kg, approximately 13-20 mg/kg, approximately 14-20 mg/kg, approximately 15-20 mg/kg, approximately 16-20 mg/kg, approximately 17-20 mg/kg, approximately 18-20 mg/kg, approximately 19-20 mg/kg, approximately 3-19 mg/kg, approximately 3-18 mg/kg, approximately 3-17 mg/kg, approximately 3-16 mg/kg, approximately 3-15 mg/kg, approximately 3-14 mg/kg, approximately 3-13 mg/kg, approximately 3-12 mg/kg, approximately 3-11 mg/kg, approximately 3-10 mg/kg, approximately 3-9 mg/kg, approximately 3-8 mg/kg, approximately 3-7 mg/kg, approximately 3-6 mg/kg, approximately 3-5 mg/kg, or approximately 3-4 mg/kg, or approximately 5-10 mg/kg. In some embodiments, a therapeutically effective dose is about 5 mg/kg. In some embodiments, a therapeutically effective dose is about 10 mg/kg.

In some embodiments, the therapeutically effective dose is equal to or greater than 50 mg/kg, 100 mg/kg, 150 mg/kg, 200 mg/kg, or 250 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 450 mg/kg, 500 mg/kg, 550 mg/kg, 600 mg/kg, 650 mg/kg, 700 mg/kg, 710 mg/kg, 720 mg/kg, 730 mg/kg, 740 mg/kg, 750 mg/kg, 800 mg/kg, 850 mg/kg, 900 mg/kg, 950 mg/kg, or 1000 mg/kg.

In some embodiments, a therapeutically effective dose is approximately 50-1,000 mg/kg, approximately 100-1,000 mg/kg, approximately 150-1,000 mg/kg, approximately 200-1,000 mg/kg, approximately 250-1,000 mg/kg, approximately 300-1,000 mg/kg, approximately 350-1,000 mg/kg, approximately 400-1,000 mg/kg, approximately 450-1,000 mg/kg, approximately 500-1,000 mg/kg, approximately 550-1,000 mg/kg, approximately 600-1,000 mg/kg, approximately 650-1,000 mg/kg, approximately 700-1,000 mg/kg, approximately 750-1,000 mg/kg, approximately 800-1,000 mg/kg, approximately 850-1,000 mg/kg, approximately 900-1,000 mg/kg, approximately 950-1,000 mg/kg, approximately 50-950 mg/kg, approximately 50-900 mg/kg, approximately 50-850 mg/kg, approximately 50-800 mg/kg, approximately 50-750 mg/kg, approximately 50-700 mg/kg, approximately 50-650 mg/kg, approximately 50-600 mg/kg, approximately 50-550 mg/kg, approximately 50-500 mg/kg, approximately 50-450 mg/kg, approximately 50-400 mg/kg, approximately 50-350 mg/kg, approximately 50-300 mg/kg, approximately 50-250 mg/kg, approximately 50-200 mg/kg, approximately 50-150 mg/kg, or approximately 50-100 mg/kg.

In some embodiments, administering comprises an initial bolus or loading dose, followed by at least one maintenance dose. In some embodiments, the initial bolus or loading dose is greater than the at least one maintenance dose. In some embodiments, the initial bolus or loading dose is at least one-fold, two-fold, three-fold, four fold or five-fold greater in dosage than the dosage of the at least one maintenance dose. In some embodiments, the initial bolus or loading dose is two-fold greater in dosage than the dosage of the at least one maintenance dose. For example, in some embodiments, the initial bolus or loading dose is 720 mg and the maintenance dose is 360 mg.

In some embodiments, a maintenance dose is administered after administration of the loading dose. In some embodiments, a flat dose is used as an initial bolus or loading dose and/or maintenance dose. In some embodiments, a suitable flat dose is provided in a single injection syringe. A suitable flat dose may be administered (e.g., subcutaneously or intravenously) in a single injection or by multiple injections. In some embodiments, a suitable flat dose is about between 10 mg and 800 mg. Accordingly, in some embodiments, a suitable flat dose is equal to or greater than about 10 mg, 20 mg, 30 mg, 40 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385, 390 mg, 395 mg, 400 mg, 405 mg, 410 mg, 415, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 or 800 mg. In some embodiments, a suitable flat dose ranges from 50-800 mg, 50-700 mg, 50-600 mg, 50-500 mg, 100-800 mg, 100-700 mg, 100-600 mg, 100-500 mg, 100-500 mg, 100-400 mg, 150-400 mg, 200-400 mg, 250-400 mg, 300-350 mg, 320-400 mg, or 350-400 mg. In some embodiments, a loading dose is about 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800. In some embodiments, a suitable initial bolus flat dose is 720 mg. In some embodiments, a maintenance dose is about 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 380 mg, 390 mg, 395 mg, or 400 mg. In some embodiments, a suitable maintenance flat dose is 360 mg. In some embodiments, the flat dose is 720 mg initial bolus dose, and is 360 mg maintenance dose. In some embodiments an initial loading or bolus dose of about 720 mg is administered. In some embodiments, the therapeutically effective dose comprises an initial bolus or loading dose of about 720 mg, followed by at least one maintenance dose of about 360 mg.

In some embodiments, a weight-based dose is used as an initial bolus or loading dose and/or maintenance dose. In some embodiments, the dose is provided in a single injection syringe. The dose may be administered (e.g., subcutaneously or intravenously) in a single injection or by multiple injections. In some embodiments, a loading dose is about 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In some embodiments, a loading dose is about between 5 mg/kg and 25 mg/kg and a maintenance dose is about between 2.5 mg/kg and 7.5 mg/kg. In some embodiments, the maintenance dose is about 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.0 mg/kg, 6.5 mg/kg, 7.0 mg/kg, or 7.5 mg/kg. In some embodiments an initial loading or bolus dose of about 10 mg/kg is administered. In some embodiments, the therapeutically effective dose comprises an initial bolus dose of about 10 mg/kg, followed by at least one maintenance dose of about 5 mg/kg.

Administration Interval

An administration interval of an anti-OSMRβ antibody in the treatment of prurigo nodularis in a subject can occur at various durations. In some embodiments of the invention, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

Treatment Period

A treatment period of prurigo nodularis with an anti-OSMRβ antibody can vary in duration. In some embodiments, the treatment period is at least one month. In some embodiments the treatment period is at least 4 weeks, or at least 5 weeks, or at least 6 weeks, or at least 7 weeks, or at least 8 weeks, or at least 9 weeks, or at least 10 weeks, or at least 11 weeks or at least 12 weeks, or at least 13 weeks, or at least 15 weeks, or at least 18 weeks, or at least 20 weeks, or at least 22 weeks, or at least 24 weeks. In some embodiments, the treatment period is at least two months. In some embodiments, the treatment period is at least three months. In some embodiments, the treatment period is at least six months. In some embodiments, the treatment period is at least nine months. In some embodiments, the treatment period is at least one year. In some embodiments, the treatment period is at least two years. In some embodiments, the treatment period is for as long as the subject is on hemodialysis.

Pharmacokinetics and Pharmacodynamics

Evaluation of anti-OSMRβ antibody concentration-time profiles in serum of subjects with prurigo nodularis may be evaluated directly by measuring systemic serum anti-OSMRβ antibody concentration-time profiles. Typically, anti-OSMRβ antibody pharmacokinetic and pharmacodynamic profiles are evaluated by sampling the blood of treated subjects periodically. The following standard abbreviations are used to represent the associated pharmacokinetic parameters.
Cₘₐₓ maximum concentration
tₘₐₓ time to maximum concentration
AUC₁₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λz is the apparent terminal elimination rate constant
λz apparent terminal elimination rate constant, where λz is the magnitude of the slope of the linear regression of the log concentration versus time profile during the terminal phase
t_{1/2} apparent terminal elimination half-life (whenever possible), where t_{1/2}=natural log (ln)(2)/λz
CL clearance
Vd volume of distribution (IV doses only)
Vd/F apparent volume of distribution (SC doses only)

Typically, actual blood sample collection times relative to the start of anti-OSMRβ antibody administration are used in PK analysis. For example, blood samples are typically collected within 15 or 30 minutes prior to anti-OSMRβ antibody administration (pre-injection baseline or time 0) and at hours 1, 4, 8 or 12, or days 1 (24 and 28 hours), 2, 3, 4, 5, 6, 7, 10, 13, 17, 20, 24, 27, 31, 34, 41, 48, 55, 62, 69, 76, 90, following administration.

Various methods may be used to measure anti-OSMRβ antibody concentration in serum. As a non-limiting example, enzyme-linked immunosorbent assay (ELISA) methods are used.

Pharmacokinetic parameters may be evaluated at any stage during the treatment, for example, at day 1, day 2, day 3, day 4, day 5, day 6, week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or later. In some embodiments, pharmacokinetic parameters may be evaluated at month 1, month 2, month 3, month 4, month 5, month 6, month 7, month 8, month 9, month 10, month 11, month 12, month 13, month 14, month 15, month 16, month 17, month 18, month 19, month 20, month 21, month 22, month 23, month 24, or later during the treatment.

Adverse Effects

Adverse effects related to the treatment of prurigo nodularis can include peripheral edema, nasopharyngitis, upper respiratory tract infections, increased creatine phosphokinase, conjunctivitis, blepharitis, oral herpes, keratitis, eye pruritus, other herpes simplex virus infection, and dry eye.

In some embodiments, administration of an anti-OSMRβ antibody results in no serious adverse effects in the subject. In some embodiments, administration of an anti-OSMRβ antibody does not result in one or more of peripheral edema, nasopharyngitis, upper respiratory tract infections, and increased creatine phosphokinase.

Combination Therapy

In some embodiments, an anti-OSMRβ antibody described herein may be used in combination with one or more other therapeutic agents for the treatment of prurigo nodularis (PN.) For example, an anti-OSMRβ antibody may be administered in combination with one or more of concomitant corticosteroids (e.g., TCS), calcineurin inhibitors, antimicrobials and/or antiseptics, antihistamines, and others (e.g., coal tar, phosphodiesterase inhibitors) that are administered systemically (e.g., orally) or topically. In some embodiments, an anti-OSMRβ antibody and one or more other therapeutic agents may be administered simultaneously. In some embodiments, an anti-OSMRβ antibody and one or more other therapeutic agents may be administered sequentially. In some embodiments, one or more other therapeutic agents may be administered as needed.

Additional Therapeutic Indications

In some embodiments the present invention provides methods and compositions for use in treating pruritus associated with Chronic Idiopathic Pruritus (CIP). In some embodiments, the method and compositions of the invention are contemplated for use in the treatment of pruritus associated with Chronic Idiopathic Urticaria (CIU). In some embodiments, the method and compositions of the invention are contemplated for use in the treatment of pruritus associated with Chronic Spontaneous Urticaria (CSU). In some embodiments, the method and compositions of the invention are contemplated for use in the treatment of pruritus associated with Cutaneous Amyloidosis (CA). In some embodiments, the method and compositions of the invention are contemplated for use in the treatment of pruritus associated with Plaque Psoriasis (PPs). In some embodiments, the method and compositions of the invention are contemplated for use in the treatment of pruritus associated with Lichen Simplex Chronicus (LSC). In some embodiments, the method and compositions of the invention are contemplated for use in the treatment of pruritus associated with Lichen Planus (LP). In some embodiments, the method and compositions of the invention are contemplated for use in the treatment of pruritus associated with Inflammatory Ichthyosis (II). In some embodiments, the method and compositions of the invention are contemplated for use in the treatment of pruritus associated with Mastocytosis (MA). In some embodiments, the method and compositions of the invention are contemplated for use in the treatment of pruritus associated with Bullous Pemphigoid (BP).

The method of treating CIP, CIU, CSU, CA, PPs, LSC, LP, MA or BP comprises administering to the subject in need of treatment an anti-OSMRβ antibody at a therapeutically effective dose and an administration interval for a treatment period sufficient to improve, stabilize or reduce one or more symptoms of pruritus relative to a control. There are several different methods for assessing symptoms of CIP, CIU, CSU, CA, PPs, LSC, LP, MA or BP. In some embodiments, one or more symptoms of any of these pruritic conditions are assessed by a change or percent change from baseline in weekly average of Worst Itch-Numeric Rating Scale (WI-NRS). In some embodiments, one or more symptoms of any of these pruritic conditions are assessed by the proportion of subjects achieving at least a 4-point reduction from baseline in weekly average WI-NRS. In some embodiments, one or more symptoms of any of these pruritic conditions are assessed by a change or percent change from baseline in pruritus Visual Analog Scale (VAS). In some embodiments, one or more symptoms of any of these pruritic conditions are assessed by change from baseline in 5-D Pruritus total score. In some embodiments, one or more symptoms of any of these pruritic conditions are assessed by change from baseline in Sleep Loss VAS. In some embodiments, one or more symptoms of any of these pruritic conditions are assessed by change from baseline in weekly average of difficulty falling asleep NRS. In some embodiments, one or more symptoms of any of these pruritic conditions are assessed by change from baseline in weekly average of sleep quality NRS. In some embodiments, one or more symptoms of any of these pruritic conditions are assessed by change from baseline in quality of life measures over time. In some embodiments, one or more symptoms of CIU or CSU are assessed by a change from baseline in weekly itch severity score, a component of Urticaria Activity Score 7 (UAS7). In some embodiments, one or more symptoms of CIU or CSU are assessed by a change from baseline in weekly hive severity score, a component of UAS7. In some embodiments, one or more symptoms of CIU or CSU are assessed by a change from baseline in UAS7.

In some embodiments, the effect of an anti-OSMRβ antibody on CIP, CIU, CSU, CA, PPs, LSC, LP, MA or BP is measured relative to a control. In some embodiments, a control is indicative of the one or more symptoms of these pruritic conditions in the subject before the treatment, including, for example, a score on a pruritus NRS greater than or equal to 5. In some embodiments, one or more symptoms of these pruritic conditions in a subject before treatment comprises a score on a pruritus NRS greater than or equal to 7. In some embodiments, a control is indicative of the one or more symptoms of these pruritic conditions in a control subject with the same disease status without treatment. In some embodiments, the control is indicative of the one or more symptoms of these pruritic conditions in a control subject with the same disease status that was administered a placebo.

In some embodiments, CIP, CIU, CSU, CA, PPs, LSC, LP, MA or BP is treated by administering to a subject in need of treatment an anti-OSMRβ antibody at a therapeutically effective dose and an administration interval for a treatment period sufficient to improve, stabilize or reduce one or more symptoms of CIP, CIU, CSU, CA, PPs, LSC, LP, MA or BP relative to a control. The terms, “treat” or “treatment,” as used in the context of CIP, CIU, CSU, CA, PPs, LSC, LP, MA or BP herein, refers to amelioration of one or more symptoms associated with CIP, CIU, CSU, CA, PPs, LSC, LP, MA or BP, prevention or delay of the onset of one or more symptoms of CIP, CIU, CSU, CA, PPs, LSC, LP, MA or BP, and/or lessening of the severity or frequency of one or more symptoms of CIP, CIU, CSU, CA, PPs, LSC, LP, MA or BP. In some embodiments, the terms, “treat” or “treatment,” as used in the context of CIP, CIU, CSU, CA, PPs, LSC, LP, MA or BP herein, refers to partially or completely alleviate, ameliorate, relieve, inhibit, prevent, delay onset of, reduce severity of and/or reduce incidence of one or more symptoms or features of CIP, CIU, CSU, CA, PPs, LSC, LP, MA or BP. In some embodiments, the administration of an anti-OSMRβ antibody results in a statistically-significant drop on a quantitative numerical pruritus scale. In some embodiments, the administration of an anti-OSMRβ antibody results in a statistically-significant drop in weekly average Worst Itch-Numerical Rating Scale (WI-NRS). In some embodiments, the weekly average WI-NRS score has at least a 4-point reduction from baseline. In some embodiments, the administration of an anti-OSMRβ antibody results in a statistically-significant drop or percent change from baseline in pruritus Visual Analog Scale (VAS). In some embodiments, the administration of an anti-OSMRβ antibody results in a statistically-significant drop or percent change from baseline in 5-D Pruritus total score. In some embodiments, the administration of an anti-OSMRβ antibody results in a statistically-significant drop or percent change from baseline in Sleep Loss VAS. In some embodiments, the administration of an anti-OSMRβ antibody results in a statistically-significant drop or percent change in weekly average of difficulty falling asleep NRS. In some embodiments, the administration of an anti-OSMRβ antibody results in a statistically-significant increase or percent change in quality of life measures over time. In some embodiments, the administration of an anti-OSMRβ antibody results in a statistically-significant drop or percent change in UAS7 score.

In some embodiments, the step of administering comprises subcutaneous administration. In some embodiments, subcutaneous administration is through subcutaneous injection. In some embodiments, subcutaneous administration is through a subcutaneous pump.

A therapeutically effective dose of an anti-OSMRβ antibody for treating CIP, CIU, CSU, CA, PPs, LSC, LP, MA or BP can occur at various dosages. In some embodiments of the invention, a therapeutically effective dose is equal to or greater than about 0.1 mg/kg, 0.2 mg/kg, 0.3 mg/kg, 0.4 mg/kg, 0.5 mg/kg, 0.6 mg/kg, 0.8 mg/kg, 0.9 mg/kg, 1 mg/kg, 1.2 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 17 mg/kg, 18 mg/kg, 19 mg/kg or 20 mg/kg, or 30 mg/kg. In some embodiments, a therapeutically effective dose is equal to or greater than 3 mg/kg, 4 mg/kg, 5 mg/kg, 6 mg/kg, 7 mg/kg, 7.5 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg.

In some embodiments a therapeutically effective dose is approximately 0.1-20 mg/kg, approximately 0.3-20 mg/kg, approximately 0.5-20 mg/kg, approximately 0.75-20 mg/kg, approximately 1-20 mg/kg, approximately 1.5-20 mg/kg, approximately 2-20 mg/kg, approximately 2.5-20 mg/kg, approximately 3-20 mg/kg, approximately 3.5-20 mg/kg, approximately 4-20 mg/kg, approximately 4.5-20 mg/kg, approximately 5-20 mg/kg, approximately 5.5-20 mg/kg, approximately 6-20 mg/kg, approximately 6.5-20 mg/kg, approximately 7-20 mg/kg, approximately 7.5-20 mg/kg, approximately 8-20 mg/kg, approximately 8.5-20 mg/kg, approximately 9-20 mg/kg, approximately 9.5-20 mg/kg, approximately 10-20 mg/kg, approximately 10.5-20 mg/kg.

In some embodiments, a therapeutically effective dose is approximately 3-20 mg/kg, approximately 4-20 mg/kg, approximately 5-20 mg/kg, approximately 6-20 mg/kg, approximately 7-20 mg/kg, approximately 8-20 mg/kg, approximately 9-20 mg/kg, approximately 10-20 mg/kg, approximately 11-20 mg/kg, approximately 12-20 mg/kg, approximately 13-20 mg/kg, approximately 14-20 mg/kg, approximately 15-20 mg/kg, approximately 16-20 mg/kg, approximately 17-20 mg/kg, approximately 18-20 mg/kg, approximately 19-20 mg/kg, approximately 3-19 mg/kg, approximately 3-18 mg/kg, approximately 3-17 mg/kg, approximately 3-16 mg/kg, approximately 3-15 mg/kg, approximately 3-14 mg/kg, approximately 3-13 mg/kg, approximately 3-12 mg/kg, approximately 3-11 mg/kg, approximately 3-10 mg/kg, approximately 3-9 mg/kg, approximately 3-8 mg/kg, approximately 3-7 mg/kg, approximately 3-6 mg/kg, approximately 3-5 mg/kg, or approximately 3-4 mg/kg, or approximately 5-10 mg/kg. In some embodiments, a therapeutically effective dose is about 5 mg/kg. In some embodiments, a therapeutically effective dose is about 10 mg/kg.

In some embodiments, the therapeutically effective dose is equal to or greater than 50 mg/kg, 100 mg/kg, 150 mg/kg, 200 mg/kg, or 250 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 450 mg/kg, 500 mg/kg, 550 mg/kg, 600 mg/kg, 650 mg/kg, 700 mg/kg, 710 mg/kg, 720 mg/kg, 730 mg/kg, 740 mg/kg, 750 mg/kg, 800 mg/kg, 850 mg/kg, 900 mg/kg, 950 mg/kg, or 1000 mg/kg.

In some embodiments, a therapeutically effective dose is approximately 50-1,000 mg/kg, approximately 100-1,000 mg/kg, approximately 150-1,000 mg/kg, approximately 200-1,000 mg/kg, approximately 250-1,000 mg/kg, approximately 300-1,000 mg/kg, approximately 350-1,000 mg/kg, approximately 400-1,000 mg/kg, approximately 450-1,000 mg/kg, approximately 500-1,000 mg/kg, approximately 550-1,000 mg/kg, approximately 600-1,000 mg/kg, approximately 650-1,000 mg/kg, approximately 700-1,000 mg/kg, approximately 750-1,000 mg/kg, approximately 800-1,000 mg/kg, approximately 850-1,000 mg/kg, approximately 900-1,000 mg/kg, approximately 950-1,000 mg/kg, approximately 50-950 mg/kg, approximately 50-900 mg/kg, approximately 50-850 mg/kg, approximately 50-800 mg/kg, approximately 50-750 mg/kg, approximately 50-700 mg/kg, approximately 50-650 mg/kg, approximately 50-600 mg/kg, approximately 50-550 mg/kg, approximately 50-500 mg/kg, approximately 50-450 mg/kg, approximately 50-400 mg/kg, approximately 50-350 mg/kg, approximately 50-300 mg/kg, approximately 50-250 mg/kg, approximately 50-200 mg/kg, approximately 50-150 mg/kg, or approximately 50-100 mg/kg.

In some embodiments, administering comprises an initial bolus or loading dose, followed by at least one maintenance dose. In some embodiments, the initial bolus or loading dose is greater than the at least one maintenance dose. In some embodiments, the initial bolus or loading dose is at least one-fold, two-fold, three-fold, four fold or five-fold greater in dosage than the dosage of the at least one maintenance dose. In some embodiments, the initial bolus or loading dose is two-fold greater in dosage than the dosage of the at least one maintenance dose. For example, in some embodiments, the initial bolus or loading dose is 720 mg and the maintenance dose is 360 mg.

In some embodiments, a maintenance dose is administered after administration of the loading dose. In some embodiments, a flat dose is used as an initial bolus or loading dose and/or maintenance dose. In some embodiments, a suitable flat dose is provided in a single injection syringe. A suitable flat dose may be administered (e.g., subcutaneously or intravenously) in a single injection or by multiple injections. In some embodiments, a suitable flat dose is about between 10 mg and 800 mg. Accordingly, in some embodiments, a suitable flat dose is equal to or greater than about 10 mg, 20 mg, 30 mg, 40 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 140 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385, 390 mg, 395 mg, 400 mg, 405 mg, 410 mg, 415, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 or 800 mg. In some embodiments, a suitable flat dose ranges from 50-800 mg, 50-700 mg, 50-600 mg, 50-500 mg, 100-800 mg, 100-700 mg, 100-600 mg, 100-500 mg, 100-500 mg, 100-400 mg, 150-400 mg, 200-400 mg, 250-400 mg, 300-350 mg, 320-400 mg, or 350-400 mg. In some embodiments, a loading dose is about 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800. In some embodiments, a suitable initial bolus flat dose is 720 mg. In some embodiments, a maintenance dose is about 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 380 mg, 390 mg, 395 mg, or 400 mg. In some embodiments, a suitable maintenance flat dose is 360 mg. In some embodiments, the flat dose is 720 mg initial bolus dose, and is 360 mg maintenance dose. In some embodiments an initial loading or bolus dose of about 720 mg is administered. In some embodiments, the therapeutically effective dose comprises an initial bolus or loading dose of about 720 mg, followed by at least one maintenance dose of about 360 mg.

In some embodiments, a weight-based dose is used as an initial bolus or loading dose and/or maintenance dose. In some embodiments, the dose is provided in a single injection syringe. The dose may be administered (e.g., subcutaneously or intravenously) in a single injection or by multiple injections. In some embodiments, a loading dose is about 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In some embodiments, a loading dose is about between 5 mg/kg and 25 mg/kg and a maintenance dose is about between 2.5 mg/kg and 7.5 mg/kg. In some embodiments, the maintenance dose is about 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.0 mg/kg, 6.5 mg/kg, 7.0 mg/kg, or 7.5 mg/kg. In some embodiments an initial loading or bolus dose of about 10 mg/kg is administered. In some embodiments, the therapeutically effective dose comprises an initial bolus dose of about 10 mg/kg, followed by at least one maintenance dose of about 5 mg/kg. An administration interval of an anti-OSMRβ antibody in the treatment of CIP, CIU, CSU, CA, PPs, LSC, LP, MA or BP in a subject can occur at various durations. In some embodiments of the invention, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

A treatment period of CIP, CIU, CSU, CA, PPs, LSC, LP, MA or BP with an anti-OSMRβ antibody can vary in duration. In some embodiments, the treatment period is at least one month. In some embodiments the treatment period is at least 4 weeks, or at least 5 weeks, or at least 6 weeks, or at least 7 weeks, or at least 8 weeks, or at least 9 weeks, or at least 10 weeks, or at least 11 weeks or at least 12 weeks, or at least 13 weeks, or at least 15 weeks, or at least 18 weeks, or at least 20 weeks, or at least 22 weeks, or at least 24 weeks. In some embodiments, the treatment period is at least two months. In some embodiments, the treatment period is at least three months. In some embodiments, the treatment period is at least six months. In some embodiments, the treatment period is at least nine months. In some embodiments, the treatment period is at least one year. In some embodiments, the treatment period is at least two years. In some embodiments, the treatment period is for as long as the subject is on hemodialysis.

Adverse effects related to the treatment of CIP, CIU, CSU, CA, PPs, LSC, LP, MA or BP can include peripheral edema, nasopharyngitis, upper respiratory tract infections, increased creatine phosphokinase, conjunctivitis, blepharitis, oral herpes, keratitis, eye pruritus, other herpes simplex virus infection, dry eye, pain, fatigue, arthralgia, fracture, leg pain, arm pain, dizziness, pruritus dermatitis, earache, and anaphalaxis presenting as bronchospasm, hypotension, syncope, urticaria, and/or angioedema of the throat or tongue.

In some embodiments, administration of an anti-OSMRβ antibody results in no serious adverse effects in the subject. In some embodiments, administration of an anti-OSMRβ antibody does not result in one or more of peripheral edema, nasopharyngitis, upper respiratory tract infections, increased creatine phosphokinase, conjunctivitis, blepharitis, oral herpes, keratitis, eye pruritus, other herpes simplex virus infection, dry eye, pain, fatigue, arthralgia, fracture, leg pain, arm pain, dizziness, pruritus dermatitis, earache, and anaphalaxis presenting as bronchospasm, hypotension, syncope, urticaria, and/or angioedema of the throat or tongue.

In some embodiments, an anti-OSMRβ antibody described herein is used in treating pruritus associated with Chronic Idiopathic Pruritus (CIP). In some embodiments, the methods of the invention are used for treating pruritus in a subject having CIP. The studies presented herein show that OSMRβ mRNA levels are increased in subjects who have CIP in comparison to subjects who do not have CIP. The method of treating CIP comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Chronic Spontaneous Urticaria (CSU), also known as Chronic Idiopathic Urticaria (CIU). In some embodiments, the methods of the invention are used for treating pruritus in a subject having CSU. The studies presented herein show that OSMRβ mRNA and protein expression levels are increased in subjects who have CSU in comparison to subjects who do not have CSU. The method of treating CSU comprises administering to the subject in need of treatment an anti-OSMRβ antibody at a therapeutically effective dose and an administration interval for a treatment period sufficient to improve, stabilize or reduce one or more symptoms of pruritus relative to a control. The method of treating CSU comprises administering to the subject in need of treatment an anti-OSMRβ antibody at a therapeutically effective dose and an administration interval for a treatment period sufficient to improve, stabilize or reduce urticaria relative to a control. In some embodiments, one or more symptoms of CSU are assessed by a change from baseline in UAS7, including, for example, itch or hives severity score. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Chronic Idiopathic Urticaria (CIU). In some embodiments, the methods of the invention are used for treating pruritus in a subject having CIU. The studies presented herein show that OSMRβ mRNA and protein expression levels are increased in subjects who have CIU in comparison to subjects who do not have CIU. The method of treating CIU comprises administering to the subject in need of treatment an anti-OSMRβ antibody at a therapeutically effective dose and an administration interval for a treatment period sufficient to improve, stabilize or reduce one or more symptoms of pruritus relative to a control. The method of treating CIU comprises administering to the subject in need of treatment an anti-OSMRβ antibody at a therapeutically effective dose and an administration interval for a treatment period sufficient to improve, stabilize or reduce urticaria relative to a control. In some embodiments, one or more symptoms of CIU are assessed by a change from baseline in UAS7, including, for example, itch or hives severity score. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Cutaneous Amyloidosis (CA). In some embodiments, the methods of the invention are used for treating pruritus in a subject having CA. The method of CA comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Lichen Simplex Chronicus (LSC). In some embodiments, the methods of the invention are used for treating pruritus in a subject having LSC. The studies presented herein show that OSMRβ mRNA expression levels are increased in subjects who have LSC in comparison to subjects who do not have LSC. The method of treating LSC comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Plaque Psoriasis (PPs). In some embodiments, the methods of the invention are used for treating pruritus in a subject having PPs. The method of PPs comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection

In some embodiments, an anti-OSMRβ antibody described herein is used in treating pruritus associated with Lichen Planus (LP). In some embodiments, the methods of the invention are used for treating pruritus in a subject having LP. The studies presented herein show that OSMRβ mRNA expression levels are increased in subjects who have LP in comparison to subjects who do not have LP. The method of treating LP comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Inflammatory Ichthyosis (II). In some embodiments, the methods of the invention are used for treating pruritus in a subject having II. The method of treating II comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Mastocytosis (MA). In some embodiments, the methods of the invention are used for treating pruritus in a subject having MA. The method of treating MA comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating pruritus associated with Bullous Pemphigoid (BP). In some embodiments, the methods of the invention are used for treating pruritus in a subject having BP. The method of treating BP comprises administering to the subject in need of treatment an anti-OSMRβ antibody at a therapeutically effective dose and an administration interval for a treatment period sufficient to improve, stabilize or reduce one or more symptoms of pruritus relative to a control. In some embodiments, the step of administering comprises subcutaneous administration. In some embodiments, the step of administering comprises intravenous administration. In some embodiments, the step of administering comprises intravenous administration followed by subcutaneous administration. In some embodiments, the subcutaneous administration is through subcutaneous injection.

In some embodiments, an anti-OSMRβ antibody described herein is used in treating a T_{H}2-mediated inflammatory disease. Oncostatin M (OSM), a member of the gp130 cytokine family, is involved in T_{H}2 inflammation, epidermal integrity, and fibrosis. In some embodiments, OSM signaling is independent of IL-31. The antibody can inhibit OSM-mediated pathways where OSM interacts with other signaling pathways, for example, IL-4 mediated pathway, IL-6 mediated pathway, IL-8 mediated pathway, IL-13 mediated pathway, and others. In some embodiments, the anti-OSMRβ antibody described herein is used in combination with inhibitors of one or more signaling members of the T_{H}2 mediated inflammatory pathways.

An effective dose, administration interval or treatment period for the above embodiments are as disclosed elsewhere in the application.

In some embodiments, a subject who has CIP, CIU, CSU, CA, PPs, LSC, LP, MA or BP has elevated levels of one or more cytokines associated with the OSMRβ signaling pathway in comparison to a healthy subject. Accordingly, in some embodiments, the subject has elevated levels of one or more of IL-31, OSM, IL-31Rα, and OSMRβ in comparison to a healthy subject. In some embodiments, the subject has elevated levels of one or more of IL-31 in comparison to a healthy subject. In some embodiments, the subject has elevated levels of one or more of OSM in comparison to a healthy subject. In some embodiments, the subject has elevated levels of one or more of IL-31Rα in comparison to a healthy subject. In some embodiments, the subject has elevated levels of one or more of OSMRβ in comparison to a healthy subject.

In some embodiments, treating a subject who has CIP, CIU, CSU, CA, PPs, LSC, LP, MA or BP results in a decrease or stabilization of MCP-1/CCL2 levels in the subject. Accordingly, in some embodiments, treating the subject results in a decrease of MCP-1 levels in comparison to the diseased state. In some embodiments, treating the subject results in stabilization of MCP-1 levels. By “stabilization” is meant that the levels of MCP-1 remain about the same and do not increase or decrease. In some embodiments, treating the subject results in reduced MCP-1 levels in lymphocytes and/or endothelial cells.

In some embodiments, the subject who has CIP, CIU, CSU, CA, PPs, LSC, LP, MA or BP has WI-NRS scores of about 4, about 5, about 6, about 7, about 8 or above. Accordingly, in some embodiments, the subject in need of treatment has WI-NRS score of about 4. In some embodiments, the subject in need of treatment has WI-NRS score of about 5. In some embodiments, the subject in need of treatment has WI-NRS score of about 6. In some embodiments, the subject in need of treatment has WI-NRS score of about 7. In some embodiments, the subject in need of treatment has WI-NRS score of about 8. In some embodiments, the subject in need of treatment has WI-NRS score of more than 8.

In some embodiments, a subject who has CIP, CIU, CSU, CA, PPs, LSC, LP, MA or BP is selected for treatment who has MCP-1/CCL2 levels greater than found in a healthy individual. In some embodiments, the subject selected for treatment does not have elevated levels of MCP-1/CCL2 in comparison to a healthy individual. In some embodiments, IL-31 expression level is elevated in the subject relative to a control. In some embodiments, IL-31 expression level is not elevated in the subject relative to a control. In some embodiments, IL-31 expression level in a portion of the subject's skin affected by a pruritic disease or condition is approximately the same as the IL-31 expression level in (i) a portion of the subject's skin that is unaffected by the pruritic disease or condition, or (ii) a portion of normal skin from a healthy subject, who is not diagnosed with a pruritic disease or condition. In some embodiments, IL-31Rα expression level is elevated in the subject relative to a control. In some embodiments, OSM expression level is elevated in the subject relative to a control. In some embodiments, OSMRβ expression level is elevated in the subject relative to a control. In some embodiments, OSMRβ expression level is not elevated in the subject relative to a control. In some embodiments, OSMRβ expression level in a portion of the subject's skin affected by a pruritic disease or condition is approximately the same as the OSMRβ expression level in (i) a portion of the subject's skin that is unaffected by the pruritic disease or condition, or (ii) a portion of normal skin from a healthy subject, who is not diagnosed with a pruritic disease or condition.

### Anti-Oncostatin M Receptor (OSMR) Antibodies

In some embodiments, inventive compositions and methods provided by the present invention are used to deliver an anti-OSMRβ antibody to a subject in need. In certain embodiments of the invention, the anti-OSMRβ antibodies are fully-human monoclonal antibodies that specifically inhibit IL-31 and oncostatin M (OSM)-induced activation of the IL-31 receptor and type II OSM receptor, respectively, through binding to OSMRB, the subunit common to both receptors. In certain embodiments, the antibody is comprised of two light chains and two heavy chains. In some embodiments, the light chain contains a lambda constant region. The constant regions of the heavy chain contain the CH1, hinge, and CH2 domains of a human immunoglobulin IgG4 antibody fused to the CH3 domain of a human IgG1 antibody. In other embodiments, the heavy chain of the anti-OSMRβ antibody contains a S228P modification to improve stability and a N297Q modification to remove an N-linked glycosylation site.

Anti-OSMRβ Heavy Chain Amino Acid Sequence
(SEQ ID NO: 1)
QVQLVQSGAEVKKPGASVKVSCKASGYTFTSYEINWVRQATGQGLEWMGW
MNPNSGYTGYAQKFQGRVTMTRDTSISTAYMEMSSLRSEDTAVYYCARDI
VAANTDYYFYYGMDVWGQGTTVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREE
QFQSTYRVVSVLTVLHQDWLNGKEYKCKVSNKGLPSSIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
Anti-OSMRβ Light Chain Amino Acid Sequence
(SEQ ID NO: 2)
QSVLTQPPSASGTPGQRVTISCSGSNSNIGSNTVNWYHQLPGTAPKLLIY
NINKRPSGVPDRFSGSKSGSSASLAISGLQSEDEADYYCSTWDDSLDGVV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS
Anti-OSMRβ Heavy Chain Variable Domain Amino
Acid Sequence
(SEQ ID NO: 3)
QVQLVQSGAEVKKPGASVKVSCKASGYTFTSYEINWVRQATGQGLEWMGW
MNPNSGYTGYAQKFQGRVTMTRDTSISTAYMEMSSLRSEDTAVYYCARDI
VAANTDYYFYYGMDVWGQGTTVTVSS
Anti-OSMRβ Light Chain Variable Domain
Amino Acid Sequence
(SEQ ID NO: 4)
QSVLTQPPSASGTPGQRVTISCSGSNSNIGSNTVNWYHQLPGTAPKLLIY
NINKRPSGVPDRFSGSKSGSSASLAISGLQSEDEADYYCSTWDDSLDGVV
FGGGTKLTVLG
Anti-OSMRβ Heavy Chain Variable Domain CDR 1
(HCDR1) Amino Acid Sequence
(SEQ ID NO: 5)
SYEIN
Anti-OSMRβ Heavy Chain Variable Domain CDR 2
(HCDR2) Amino Acid Sequence
(SEQ ID NO: 6)
WMGWMNPNSGYTGYAQKFQGR
Anti-OSMRβ Heavy Chain Variable Domain CDR 3
(HCDR3) Amino Acid Sequence
(SEQ ID NO: 7)
DIVAANTDYYFYYGMDV
Anti-OSMRβ Light Chain Variable Domain CDR1
(LCDR1) Amino Acid Sequence
(SEQ ID NO: 8)
SGSNSNIGSNTVN
Anti-OSMRβ Light Chain Variable Domain CDR2
(LCDR2) Amino Acid Sequence
(SEQ ID NO: 9)
NINKRPS
Anti-OSMRβ Light Chain Variable Domain CDR3
(LCDR3) Amino Acid Sequence
(SEQ ID NO: 10)
STWDDSLDGVV
Anti-OSMRβ Heavy Chain Signal Peptide
Amino Acid Sequence
(SEQ ID NO: 11)
MDFGLSLVFLVLILKGVQC
Anti-OSMRβ Light Chain Signal Peptide
Amino Acid Sequence
(SEQ ID NO: 12)
MATGSRTSLLLAFGLLCLSWLQEGSA
Anti-OSMRβ Heavy Chain Amino Acid Sequence—
IgG4 CH1, Hinge, and CH2 Domains
(SEQ ID NO: 13)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFQSTYRVVSVLTVLHQDWLNGKEYK
CKVSNKGLPSSIEKTISKAK
Anti-OSMRβ Heavy Chain Amino Acid Sequence—
IgG1 CH3 Domain
(SEQ ID NO: 14)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG
Anti-OSMRβ Heavy Chain Amino Acid Sequence—
Constant Domain
(SEQ ID NO: 15)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFQSTYRVVSVLTVLHQDWLNGKEYK
CKVSNKGLPSSIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG
Anti-OSMRβ Light Chain Amino Acid Sequence—
IgG Lambda Constant Domain
(SEQ ID NO: 16)
QPKAAPSVTLFPPSSEELQANKATLVCLISDFYPGAVTVAWKADSSPVKA
GVETTTPSKQSNNKYAASSYLSLTPEQWKSHRSYSCQVTHEGSTVEKTVA
PTECS

In some embodiments of the invention, an anti-OSMRβ antibody comprises a light chain complementary-determining region 1 (LCDR1) defined by SEQ ID NO: 8, a light chain complementary-determining region 2 (LCDR2) defined by SEQ ID NO: 9, and a light chain complementary-determining region 3 (LCDR3) defined by SEQ ID NO: 10; and a heavy chain complementary-determining region 1 (HCDR1) defined by SEQ ID NO: 5, a heavy chain complementary-determining region 2 (HCDR2) defined by SEQ ID NO: 6, and a heavy chain complementary-determining region 3 (HCDR3) defined by SEQ ID NO: 7.

In some embodiments of the invention, an anti-OSMRβ antibody comprises CDR amino acid sequences with at least 75%, 80%, 85%, 90%, 91%, 92%, 93%, 94%, 95%, 96%, 97%, 98%, 99% or more identity with one or more of SEQ ID NO: 8, SEQ ID NO: 9, SEQ ID NO: 10, SEQ ID NO: 5, SEQ ID NO: 6, and SEQ ID NO: 7.

In some embodiments of the invention, an anti-OSMRβ antibody comprises a light chain variable domain having an amino acid sequence at least 90% identical to SEQ ID NO: 4 and a heavy chain variable domain having an amino acid sequence at least 90% identical to SEQ ID NO: 3. In some embodiments of the invention, an anti-OSMRβ antibody has a light chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 4 and a heavy chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 3. In some embodiments of the invention, an anti-OSMRβ antibody comprises a light chain variable domain that has the amino acid sequence set forth in SEQ ID NO: 4 and a heavy chain variable domain that has the amino acid sequence set forth in SEQ ID NO: 3.

In some embodiments of the invention, an anti-OSMRβ antibody comprises a light chain having an amino acid sequence at least 90% identical to SEQ ID NO: 2 and a heavy chain having an amino acid sequence at least 90% identical to SEQ ID NO: 1. In some embodiments of the invention, an anti-OSMRβ antibody has a light chain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 2 and a heavy chain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 1. In some embodiments of the invention, an anti-OSMRβ antibody comprises a light chain that has the amino acid sequence set forth in SEQ ID NO: 2 and a heavy chain that has the amino acid sequence set forth in SEQ ID NO: 1.

In some embodiments of the invention, a heavy chain constant region of an anti-OSMRβ antibody comprises CH1, hinge and CH2 domains derived from an IgG4 antibody fused to a CH3 domain derived from an IgG1 antibody. In some embodiments, the CH1, hinge and CH2 domains derived from an IgG4 antibody comprise SEQ ID NO: 13. In some embodiments, the CH3 domain derived from an IgG1 antibody comprises SEQ ID NO: 14. In some embodiments, the heavy chain constant region of an anti-OSMRβ antibody according to the present invention comprises an amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 13. In some embodiments, the heavy chain constant region of an anti-OSMRβ antibody according to the present invention comprises an amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 14. In some embodiments, the heavy chain constant region of an anti-OSMRβ antibody according to the present invention comprises an amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 15. In some embodiments, an anti-OSMRβ antibody according to the present invention comprises a lambda constant domain derived from an IgG antibody. In some embodiments, the lambda constant domain derived from an IgG comprises SEQ ID NO: 16. In some embodiments, an anti-OSMRβ antibody according to the present invention comprises an amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 16.

### REFERENCES

Bilsborough J, Leung D Y, Maurer M, Howell M, Boguniewicz M, Yao L, Storey H, LeCiel C, Harder B, Gross J A. IL-31 is associated with cutaneous lymphocyte antigen-positive skin homing T cells in patients with atopic dermatitis. J Allergy Clin Immunol. 2006 February; 117(2):418-25.
Boguniewicz M, Leung D Y. Atopic dermatitis: a disease of altered skin barrier and immune dysregulation. Immunol Rev. 2011; 242:233-46.
Boniface K, Diveu C, Morel F, Pedretti N, Froger J, Rayon E, et al. Oncostatin M secreted by skin infiltrating T lymphocytes is a potent keratinocyte activator involved in skin inflammation. J Immunol. 2007; 178(7):4615-22.
Botelho F M, Rangel-Moreno J, Fritz D, Randall T D, Xing Z, and Richards C D. Pulmonary expression of oncostatin M (OSM) promotes inducible BALT formation independently of IL-6, despite a role for IL-6 in OSM-driven pulmonary inflammation. J Immunol. 2013; 191(3):1453-64.
Brandt E B, Sivaprasad U. Th2 cytokines and atopic dermatitis. J Clin Cell Immunol 2011; 2:110.
Dillon S R, Sprecher C, Hammond A, Bilsborough J, Rosenfeld-Franklin M, Presnell S R, et al. Interleukin 31, a cytokine produced by activated T cells, induces dermatitis in mice. Nat Immunol. 2004; 5(7):752-60.
Edukulla R, Singh B, Jegga A G, Sontake V, Dillon S R, and Madala S K. Th2 Cytokines Augment IL-31/IL-31R A Interactions via STAT6-dependent IL-31R A Expression. J Biol Chem. 2015; 290(21):13510-20.
Ezzat M H, Hasan Z E, Shaheen K Y. Serum measurement of interleukin-31 (IL-31) in paediatric atopic dermatitis: elevated levels correlate with severity scoring. J Eur Acad Dermatol Venereol. 2011 March; 25(3):334-9.
Fritz D K, Kerr C, Botelho F, Stampfli M, and Richards C D. Oncostatin M (OSM) primes IL-13- and IL-4-induced eotaxin responses in fibroblasts: regulation of the type-II IL-4 receptor chains IL-4Ralpha and IL-13Ralpha1. Exp Cell Res. 2009; 315(20):3486-99.
Fritz D K, Kerr C, Fattouh R, Llop-Guevara A, Khan W I, Jordana M, et al. A mouse model of airway disease: oncostatin M-induced pulmonary eosinophilia, goblet cell hyperplasia, and airway hyperresponsiveness are STAT6 dependent, and interstitial pulmonary fibrosis is STAT6 independent. J Immunol. 2011; 186(2):1107-18.
Fritz D K, Kerr C, Tong L, Smyth D, and Richards C D. Oncostatin-M up-regulates VCAM-1 and synergizes with IL-4 in eotaxin expression: involvement of STAT6. J Immunol. 2006; 176(7):4352-60.
Gittler J K, Shemer A, Suárez-Fariñas M, et al. Progressive activation of T(H)2/T(H)22 cytokines and selective epidermal proteins characterizes acute and chronic atopic dermatitis. J Allergy Clin Immunol. 2012; 30:1344-54.
Heise R, Neis M M, Marquardt Y, Joussen S, Heinrich P C, Merk H F, et al. IL-31 receptor alpha expression in epidermal keratinocytes is modulated by cell differentiation and interferon gamma. J Invest Dermatol. 2009; 129(1):240-3.
Ip W K, Wong C K, Li M L, Li P W, Cheung P F, Lam C W. Interleukin-31 induces cytokine and chemokine production from human bronchial epithelial cells through activation of mitogen-activated protein kinase signaling pathways: implications for the allergic response. Immunology. 2007 December; 122(4):532-41.
Kanda N, Watanabe S. Increased serum human β-defensin-2 levels in atopic dermatitis: relationship to IL-22 and oncostatin M. Immunobiology. 2012 April; 217(4):436-45. Epub 2011 Oct. 23.
Kasraie S, Niebuhr M, Baumert K, Werfel T. Functional effects of interleukin 31 in human primary keratinocytes. Allergy. 2011 July; 66(7):845-52.
Ko M J, Peng Y S, Chen H Y, Hsu S P, Pai M F, Yang J Y, et al. Interleukin-31 is associated with uremic pruritus in patients receiving hemodialysis. J Am Acad Dermatol. 2014 December; 71(6):1151-1159.
Liu W, Luo R, Chen Y, Sun C, Wang J, Zhou L, et al. Interleukin-31 promotes helper T cell type-2 inflammation in children with allergic rhinitis. Pediatr Res. 2015 January; 77(1-1):20-8.
Makhlough A. Topical capsaicin therapy for uremic pruritus in patients on hemodialysis. Iran J Kidney Dis. 2010 April; 4(2):137-40.
Mozaffarian A, Brewer A W, Trueblood E S, Luzina I G, Todd N W, Atamas S P, et al. Mechanisms of oncostatin M-induced pulmonary inflammation and fibrosis. J Immunol. 2008 Nov. 15; 181(10):7243-53.
Nemoto O, Fume M, Nakagawa H, Shiramoto M, Hanada R, Matsuki S, et al. The first trial of CIM331, a humanized antihuman interleukin-31 receptor A antibody, in healthy volunteers and patients with atopic dermatitis to evaluate safety, tolerability and pharmacokinetics of a single dose in a randomized, double-blind, placebo-controlled study. Br J Dermatol. 2016 February; 174(2):296-304. Epub 2015 Dec. 19.
Niebuhr M, Mamerow D, Heratizadeh A, Satzger I, and Werfel T. Staphylococcal alpha-toxin induces a higher T cell proliferation and interleukin-31 in atopic dermatitis. Int Arch Allergy Immunol. 2011; 156(4):412-5.
Nobbe S, Dziunycz P, Muhleisen B, Bilsborough J, Dillon S R, French L E, et al. IL-31 expression by inflammatory cells is preferentially elevated in atopic dermatitis. Acta Derm Venereol. 2012; 92(1):24-8.
Pisoni R L, Wikström B, Elder S J, Akizawa T, Asano Y, Keen M L, et al. Pruritus in haemodialysis patients: International results from the Dialysis Outcomes and Practice Patterns Study (DOPPS). Nephrol Dial Transplant. 2006 December; 21(12):3495-505.
Raap U, Wichmann K, Bruder M, Ständer S, Wedi B, Kapp A, et al. Correlation of IL-31 serum levels with severity of atopic dermatitis. J Allergy Clin Immunol. 2008 August; 122(2):421-3.
Rabeony H, Petit-Paris I, Gamier J, Barrault C, Pedretti N, Guilloteau K, et al. Inhibition of keratinocyte differentiation by the synergistic effect of IL-17A, IL-22, IL-1alpha, TNFalpha and oncostatin M. PLoS One. 2014; 9(7):e101937.
Richards C D. The enigmatic cytokine oncostatin m and roles in disease. ISRN Inflamm. 2013; 2013:512103.
Ring J, Alomar A, Bieber T, Deleuran M, Fink-Wagner A, Gelmetti C, et al. Guidelines for treatment of atopic eczema (atopic dermatitis) part I. J Eur Acad Dermatol Venereol. 2012; 26:1045-60.
Ruzicka T, Hanifin J, Furue M, Pulka G, Mlynarczyk I, Wollenberg A, Galus R, Etoh T, Mihara R, Yoshida H, Stewart J, Kabashima K. Anti-Interleukin-31 Receptor A Antibody for Atopic Dermatitis. N Engl J Med 2017; 376(9):826-835.
Sidbury R, Davis D M, Cohen D E, Cordoro K M, Berger T G, Bergman J N, et al. Guidelines of care for the management of atopic dermatitis: section 3. Management and treatment with phototherapy and systemic agents. J Am Acad Dermatol. 2014; 71:327-49.
Silverberg J I, Hanifin J M. Adult eczema prevalence and associations with asthma and other health and demographic factors: a U S population-based study. J Allergy Clin Immunol 2013; 132:1132-38.
Sonkoly E, Muller A, Lauerma A I, Pivarcsi A, Soto H, Kemeny L, et al. IL-31: a new link between T cells and pruritus in atopic skin inflammation. J Allergy Clin Immunol. 2006 February; 117(2):411-7.
Stott B, Lavender P, Lehmann S, Pennino D, Durham S, and Schmidt-Weber C B. Human IL-31 is induced by IL-4 and promotes T_{H}2-driven inflammation. J Allergy Clin Immunol. 2013; 132(2):446-54 e5.

### EXAMPLES

While certain methods of the present invention have been described with specificity in accordance with certain embodiments, the following examples serve only to illustrate the methods of the invention and are not intended to limit the same.

### Example 1: Effect of Anti-OSMRβ Antibody on Cynomolgus Monkeys

The study in this example was designed to evaluate the single-dose pharmacokinetics and efficacy of an anti-OSMRβ antibody, following intravenous (IV) administration in male cynomolgus monkeys. A previous study was performed to determine the dose level of human IL-31 that produced the most consistent and robust scratching response in male cynomolgus monkeys following intradermal administration. The dose level selected was 3 μg/kg of human IL-31, which is a supra-physiologic level IL-31 cytokine.

### Experimental Design

Selection of Animals

Male cynomolgus monkeys were selected from SNBL USA stock. Selected animals were examined by veterinary staff. In addition, behavior assessments were performed prior to study start to rule out animals that might have been excessive groomers or animals with preexisting skin conditions or alopecia. Only animals that met facility health criteria and that were considered healthy were approved by a veterinarian for use in the study.

Acclimation Period

Previously quarantined animals were acclimated to the study room for a minimum of 14 days prior to initiation of dosing. Acclimation phase data was collected from all animals, including spares. During acclimation, each animal was monitored using the Noldus video monitoring system for a duration of at least 30 minutes, and the number of scratching or grooming events was recorded. Animals that had more than 40 scratching/grooming events during the 30 minute pre-screen duration were replaced with available spares and removed from the study.

Randomization

From the animals that met the specified criteria above, a stratified randomization scheme incorporating body weights was used and performed during acclimation, to assign animals to study groups.

Study Design

Four treatment groups (0, 1, 3, and 10 mg/kg of anti-OSMRβ antibody) were compared. Six animals were assigned to each treatment group. The total volume dose (mL) was calculated based on the most recent body weight.

All animals were administered the recombinant human (rh)IL-31 challenge by intradermal (ID) bolus or loading injection, using a straight needle and syringe in the exterior/lateral region of the thigh. Injection sites were shaved at least 1 day prior to dosing. Animals were dosed with the rhIL-31 challenge on Days −1, 2 (24 hours post-dosing with anti-OSMRβ antibody), 8, 15, 22, and 29 and they were dosed with the anti-OSMRβ antibody once on Day 1.

Observations and Examinations

Clinical observations were performed twice daily for each animal beginning on the second day of acclimation (Day −13). The first observation occurred in the morning, prior to room cleaning. The second observation was no sooner than four hours after the morning observation (and not during video monitoring). Additional clinical observations were performed as necessary. If clinical observations for an animal demonstrated declining animal condition, a veterinary evaluation was performed.

Detailed clinical observations/assessment of the animals was performed, while in their procedure cages, once during acclimation, and once at 5.5 hours (+10 minutes) after each rhIL-31 challenge administration. Examinations included observations of the ID injection sites, and any notable irritation or marks from scratching or grooming behaviors. All abnormalities were recorded.

On each day of rhIL-31 challenge administration, animals were monitored using the Noldus Media Recorder for at least 1 hour prior to dosing, and for a duration of at least 1 hour, beginning 30 minutes post-dose. Observations of scratching and/or self-grooming (may also include plucking at fur/skin or using teeth to pull at skin) was documented during this time. The location and the duration of each event was also documented.

If observations for an animal demonstrated declining animal condition, a veterinary evaluation was performed.

Each animal was weighed prior to the first day of dosing, and once weekly during the dosing phase. Additional body weights were taken if necessary.

Blood Collection Procedures

Blood was collected from a peripheral vein of restrained, conscious animals. Whenever possible, blood was collected via a single draw and then divided appropriately. If possible, venous blood samples were collected from conscious unscheduled animals prior to anesthesia and necropsy.

Blood samples for PK were taken 3 hours after each IL-31 challenge dose on Days −1, 2, 8, 15, 22, and 29. Approximately 1 mL of blood was taken for each sample. Single aliquots of serum were obtained after centrifugation (at 2-8° C.), transferred to appropriately sized cryovials and stored at −60 to −86° C. Specimens were stored on dry ice prior to storage.

### Results

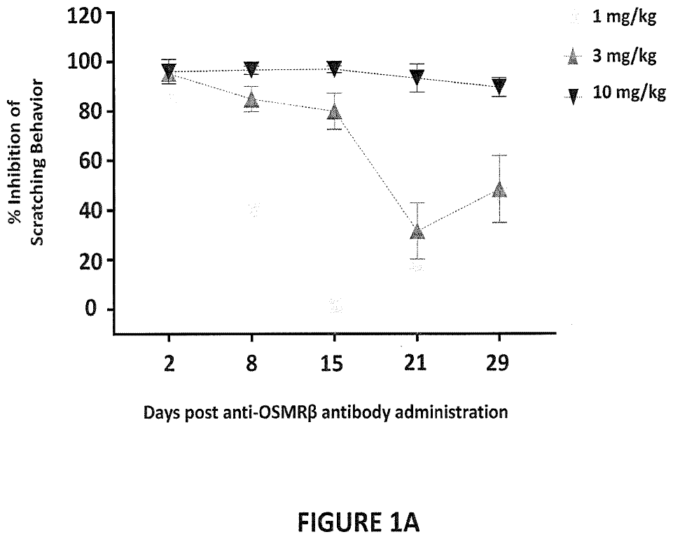

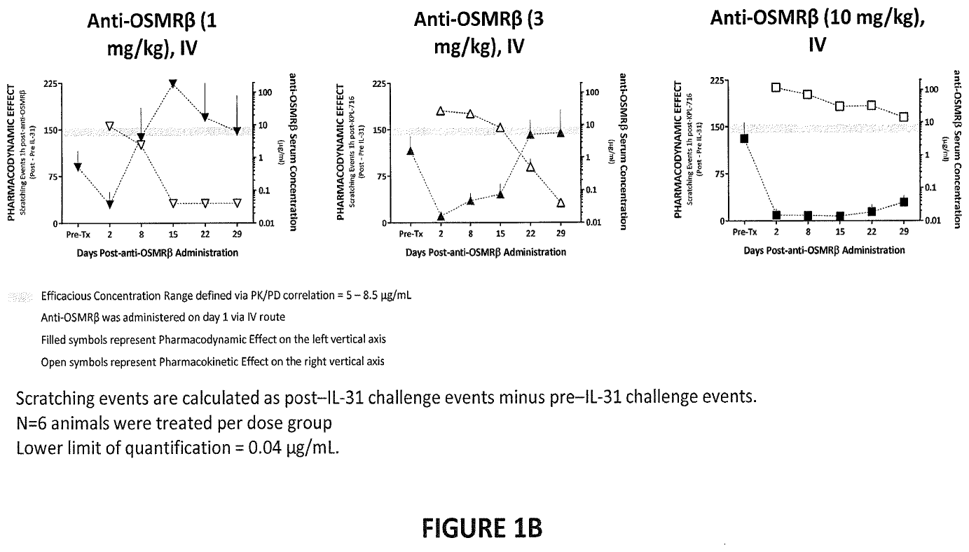

As shown in FIGS. 1A and 1B, a single IV administration of the anti-OSMRβ antibody produced a dose-dependent effect in reducing IL-31-induced scratching behavior in Cynomolgus monkeys that lasted at least as long as 29 days.

The lowest dose of anti-OSMRβ antibody tested, 1 mg/kg, produced an 86% inhibition in scratching counts 24 hours post-drug administration. This anti-pruritic effect was 40% by day 8, suggesting that the effect lasts somewhere between 1-7 days post-dose.

The middle dose of anti-OSMRβ antibody tested, 3 mg/kg, produced a 95% inhibition in scratching counts 24 hours post-drug administration. This anti-pruritic effect was 32% by day 21, suggesting that the effect lasts somewhere between 15-21 days post-dose.

The highest dose of anti-OSMRβ antibody tested, 10 mg/kg, produced a 96% μinhibition in scratching counts 24 hours post-drug administration. This anti-pruritic effect remained at 90% through day 29, suggesting the effect lasts at least as long as 29 days post-dose.

FIG. 1B shows raw scratching behavior on the left vertical axis plotted alongside serum concentration of anti-OSMRβ antibody shown on the right vertical axis. Data are shown for a single IV administration of anti-OSMRβ antibody at 1 mg/kg (left panel), 3 mg/kg (center panel) and 10 mg/kg (right panel). Results from this PK/PD correlation define a concentration range of 5 μg/ml to 8.5 μg/ml at or above which the anti-OSMRβ antibody provides protection from a supra-physiologic concentration of human IL-31-induced pruritus.

### Example 2: Treatment of Atopic Dermatitis with Anti-OSMRβ Antibody

The study in this example is designed to evaluate the safety, tolerability, PK and immunogenicity of an anti-OSMRβ antibody in subjects with atopic dermatitis. The study also includes exploratory investigations of pharmacogenetics and the effect of the anti-OSMRβ antibody on clinical effect assessments, gene expression, and PD measures.

### Study Design

An anti-OSMRβ antibody is administered intravenously (IV) to subjects with moderate to severe atopic dermatitis experiencing moderate to severe pruritus. Additionally, the anti-OSMRβ antibody is administered subcutaneously (SC) to one group of subjects with moderate to severe atopic dermatitis experiencing moderate to severe pruritus.

Subjects are enrolled into one of seven groups as described below. After verification of eligibility, subjects are randomized to receive the anti-OSMRβ antibody or placebo. In six of the groups, the anti-OSMRβ antibody or placebo is administered IV. In the seventh group, subjects receive either the anti-OSMRβ antibody or placebo as a single SC injection.

The first group receives 0.3 mg/kg anti-OSMRβ antibody or placebo intravenously. The second group receives 1.5 mg/kg anti-OSMRβ antibody or placebo intravenously. The third group receives 5 mg/kg anti-OSMRβ antibody or placebo intravenously. The fourth group receives 10 mg/kg anti-OSMRβ antibody or placebo intravenously. The fifth group receives 20 mg/kg anti-OSMRβantibody or placebo intravenously. The sixth group receives 7.5 mg/kg anti-OSMRβ antibody or placebo intravenously. The seventh group receives 1.5 mg/kg anti-OSMRβ antibody or placebo subcutaneously. Following dosing, subjects undergo at least 2 days of safety monitoring and intensive PK sampling while confined at the clinical research unit. The PK samples are collected at pre-specified timepoints.

### Study Treatments

The anti-OSMRβ antibody drug product is a sterile liquid formulation, supplied as a single use vial for IV or SC injection. 3 mL Schott vials are filled with 2.3 mL to allow for a delivered volume of 2 mL, for an extractable dose of 200 mg/vial. The anti-OSMRβ antibody drug product is diluted to a volume of 100 mL for IV infusions.

Doses administered IV are diluted in saline to a total volume of 100 mL and infused over 1 hour. Subjects are observed closely for any infusion reactions. The infusion is stopped in the event of signs and symptoms suggesting an infusion reaction. The infusion is restarted upon resolution of the signs and symptoms related to the infusion reaction. The duration of infusion can be lengthened to longer than 1 hour during the course of the study.

### Subject Inclusion Criteria

Subjects have to have a Pruritus NRS score≥7 at Screening Visit 1 and a Pruritus NRS score≥5 at check-in on Day −1. Subjects also have to have a physician-documented diagnosis of atopic dermatitis for at least 1 year and a diagnosis of moderate to severe disease, defined as IGA of 3 or 4, and body surface area (BSA) involvement of 10% or more, for at least 3 months before Screening Visit 1.

### Study Assessments

Blood samples are collected by venipuncture or cannulation, and serum concentrations of the anti-OSMRβ antibody are determined using a validated analytical procedure. The following PK parameters are calculated for each subject, whenever possible, based on the serum concentrations of the anti-OSMRβ antibody:
Cₘₐₓ maximum concentration
tₘₐₓ time to maximum concentration
AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λz is the apparent terminal elimination rate constant
λz apparent terminal elimination rate constant, where λz is the magnitude of the slope of the linear regression of the log concentration versus time profile during the terminal phase
t_{1/2} apparent terminal elimination half-life (whenever possible), where t_{1/2}=natural log (ln)(2)/λz
CL clearance
Vd volume of distribution (IV doses only)
Vd/F apparent volume of distribution (SC doses only)

Descriptive statistics (arithmetic mean, standard deviation, minimum, median, maximum, geometric mean, and geometric coefficient of variation, as appropriate) are listed and summarized for serum concentrations of anti-OSMRβ antibody and PK parameters.

Where data are available, anti-OSMRβ antibody dose proportionality is examined between the dose groups. The AUC_{0-∞}, AUC₀₋ₜ, and Cₘₐₓ estimates are tested for dose proportionality using a power model approach or analysis of variance (ANOVA) model as appropriate.

Where data are available, exposure of anti-OSMRβ antibody administered by SC injection is compared to the group that received IV administration of the same dosage. Log-transformed AUC_{0-∞} and AUC₀₋ₜ estimates are analyzed using an ANOVA model with group as a fixed effect. The ratios of geometric least squares means are calculated along with the 90% confidence interval for the ratios. Other analytical tests are employed depending on the characteristics of the dataset.

The following clinical response assessments are also conducted during the study.

Using the Pruritus Numerical Rating Scale, subjects are asked to assign a numerical score to the intensity of their pruritus symptoms using a scale from 0 to 10, with 0 indicating no pruritus and 10 indicating the worst imaginable pruritus. The NRS tool is used to assess subjects' level of pruritus at Screening Visit 1 and Day −1 to determine eligibility for the study. Subjects are instructed on daily reporting of the NRS score at Screening Visit 2, when they are provided with e-Diaries and are followed for compliance at every clinic visit. Subjects complete the rating scale daily from Screening Visit 2 to Day 60.

The Investigator's Global Assessment (IGA) is an overall assessment that is performed on each subject. The IGA utilizes a 6-point scale ranging from 0 (clear) to 5 (very severe disease). An IGA score is assigned based on morphology without referring back to the baseline state. The IGA score is recorded in the electronic Case Report Form (e-CRF). Qualified dermatologists perform IGA assessments for this study.

The Eczema Area and Severity Index (EASI) score is used to measure the severity and extent of atopic dermatitis. The 4 body regions (head and neck, trunk, upper limbs, and lower limbs) are assessed separately for erythema, infiltration/papulation, excoriation, and lichenification. The average clinical severity of each sign in each of the 4 body regions is assigned a score of 0 to 3, based on severity of disease, and the score is recorded in the e-CRF. The area of skin involved in each body region is determined and assigned a score of 0 to 6, based on extent of involvement, and the score is recorded in the e-CRF. Total EASI score at each visit is calculated at the end of the study. Qualified dermatologists perform EASI assessment for this study.

Scoring Atopic Dermatitis (SCORAD) is utilized to assess the severity of atopic dermatitis. The SCORAD is a tool used in clinical research and practice that was developed to standardize the evaluation of the extent and severity of atopic dermatitis. The SCORAD incorporates both objective physician estimates of extent and severity of disease as well as subjective subject assessment of itch and sleep loss. The percentage of each body area affected by atopic dermatitis is determined and the sum of all areas are reported. Furthermore, the severity of 6 symptoms of AD is rated as none (0), mild (1), moderate (2), or severe (3). Measures of itch and sleeplessness are included. The SCORAD is calculated based on a pre-defined formula.

Standardized medical photography is obtained of the area with the worst atopic dermatitis involvement at Screening Visit 1, Screening Visit 2, Check-in (Day −1), Day 7, Day 14, Day 28 and Day 60. The photograph(s) include the area affected by atopic dermatitis and joints on both sides of the lesion. Subject identifiable information is removed.

Body Surface Area (BSA) affected by atopic dermatitis is determined for each section of the body (head, trunk, arms and legs). The percentage of all major body areas affected is combined.

The Dermatology Life Quality Index (DLQI) is a 10-question questionnaire that takes into account symptoms and feelings, daily activities, leisure, school, personal relationships, and treatment. Each question is answered on a scale of 0 to 3 (0 for not at all, 1 for a little, 2 for a lot, and 3 for very much), taking into account the previous week. The scores are added with minimum of 0, meaning no effect on quality of life, and 30, meaning extremely large effect.

The Hospital Anxiety and Depression Scale (HADS) is a general Likert scale used to detect states of anxiety and depression. The 14 items on the questionnaire include 7 that are related to anxiety and 7 that are related to depression. Each item on the questionnaire is scored on a scale of 0 to 3 with a possible total score between 0 and 21 for each parameter.

Actigraphy utilizes a portable device (actigraphy watch) that records movement over extended periods of time. Subjects wear a wrist actigraphy watch at night on the non-dominant wrist to monitor sleep quality and quantity.

Any clinically significant worsening from baseline in subjects' signs and symptoms of atopic dermatitis is considered an adverse event (AE) (e.g., atopic dermatitis worsening/flare) and triggers consultation with the study site dermatologist, determination of the IGA (for inclusion in the e-CRF), preparation of a detailed clinical summary and reporting within 24 hours. Any changes or additions to the subject's concomitant medications are entered into the e-CRF with appropriate start and stop dates. During the study, all adverse events and severe adverse events are followed until resolution.

### Example 3: Treatment of Uremic Pruritus with Anti-OSMRβ Antibody

The study in this example is designed to evaluate the safety, tolerability, PK and immunogenicity of an anti-OSMRβ antibody in subjects on hemodialysis with uremic pruritus. The study also includes exploratory investigations of pharmacogenetics and the effect of the anti-OSMRβ antibody on clinical effect assessments, gene expression, and PD measures.

### Study Design

An anti-OSMRβ antibody is administered intravenously (IV) to subjects on hemodialysis with uremic pruritus.

Subjects are enrolled in one treatment group. After verification of eligibility, subjects are randomized to receive 5 mg/kg or 10 mg/kg of the anti-OSMRβ antibody or placebo on Day 0, the day before a regularly scheduled hemodialysis session.

Following dosing, subjects undergo at least 2 days of safety monitoring and intensive PK sampling while confined at the clinical research unit. The PK samples are collected at pre-specified timepoints. Intensive PK sampling is performed at the time of certain hemodialysis sessions. Pre- and post-dialysis blood samples as well as pre- and post-dialyzer samples and dialysate samples are collected at specified timepoints for anti-OSMRβ concentration analysis. Subjects are assessed at regular intervals through the study for additional safety monitoring, AE reporting, verification of compliance with e-Diaries, and PK sampling. At each study visit, concomitant medications (continued or new) are reviewed and recorded in the e-CRF.

### Study Treatments

Doses administered IV are diluted in saline to a total volume of 100 mL and infused over 1 hour. Subjects are observed closely for any infusion reactions. The infusion is stopped in the event of signs and symptoms suggesting an infusion reaction. The infusion is restarted upon resolution of the signs and symptoms related to the infusion reaction. The duration of infusion can be lengthened to longer than 1 hour during the course of the study.

### Subject Inclusion Criteria

Subjects have to have a Pruritus NRS score ≥7 at Screening Visit 1 and a Pruritus NRS score ≥5 at check-in on Day −1. Subjects also have to have end stage renal disease (ESRD) at Screening Visit 1 and be undergoing a three-times-per-week hemodialysis regimen that has been stable for at least 3 months before Screening Visit 1.

### Study Assessments

Blood samples are collected by venipuncture or cannulation and serum concentrations of the anti-OSMRβ antibody are determined using a validated analytical procedure. The following PK parameters are calculated for each subject, whenever possible, based on the serum concentrations of the anti-OSMRβ antibody:
Cₘₐₓ maximum concentration
tₘₐₓ time to maximum concentration
AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λz is the apparent terminal elimination rate constant
λz apparent terminal elimination rate constant, where λz is the magnitude of the slope of the linear regression of the log concentration versus time profile during the terminal phase
t_{1/2} apparent terminal elimination half-life (whenever possible), where t_{1/2}=natural log (ln)(2)/λz
CL clearance
Vd volume of distribution (IV doses only)
Vd/F apparent volume of distribution (SC doses only)

The following samples are collected for each subject on hemodialysis days designated for intensive PK sampling: blood immediately before and after the hemodialysis run; a dialysate sample; and samples from upstream and downstream of the dialyzer, urine samples before and after hemodialysis (for subjects capable of producing urine), and a 24-hour urine sample (for subjects capable of producing urine) sometime between Day 0 to Day 2 while confined at the clinical research unit. Weight and standing and supine blood pressure before and after hemodialysis are also recorded. In addition, the hemodialysis flow rate, volume of dialysate, and other hemodialysis parameters are also collected and recorded in the e-CRF. Medications given during hemodialysis are also recorded in the e-CRF. The following additional parameters are also calculated for each subject, whenever possible, based on serum and dialysate concentrations of the anti-OSMRβ antibody: dialysate clearance and dialysate extraction ratio calculated as the percentage of administered dose extracted during hemodialysis. The hemodialysis flow rate and volume of dialysate are recorded.

Descriptive statistics (arithmetic mean, standard deviation, minimum, median, maximum, geometric mean, and geometric coefficient of variation, as appropriate) are listed and summarized for serum concentrations of anti-OSMRβ antibody and PK parameters.

An exploratory analysis of the anti-OSMRβ antibody PK when administered to subjects on hemodialysis with uremic pruritus is included. Descriptive statistics are listed and summarized for serum and dialysate concentrations of the anti-OSMRβ antibody and associated PK parameters.

The following clinical response assessments are also conducted during the study.

Using the Pruritus Numerical Rating Scale (NRS), subjects are asked to assign a numerical score to the intensity of their pruritus symptoms using a scale from 0 to 10, with 0 indicating no pruritus and 10 indicating the worst imaginable pruritus. The NRS tool is used to assess subjects' level of pruritus at Screening Visit 1 and Day −1 to determine eligibility for the study. Subjects are instructed on daily reporting of the NRS score at Screening Visit 2, when they are provided with e-Diaries, and are followed for compliance at every clinic visit. Subjects complete the rating scale daily from Screening Visit 2 to Day 60.

The Dermatology Life Quality Index (DLQI) is a 10-question questionnaire that takes into account symptoms and feelings, daily activities, leisure, school, personal relationships, and treatment. Each question is answered on a scale of 0 to 3 (0 for not at all, 1 for a little, 2 for a lot, and 3 for very much), taking into account the previous week. The scores are added with minimum of 0, meaning no effect on quality of life, and 30, meaning extremely large effect.

The Hospital Anxiety and Depression Scale (HADS) is a general Likert scale used to detect states of anxiety and depression. The 14 items on the questionnaire include 7 that are related to anxiety and 7 that are related to depression. Each item on the questionnaire is scored on a scale of 0 to 3 with a possible total score between 0 and 21 for each parameter.

Actigraphy utilizes a portable device (actigraphy watch) that records movement over extended periods of time. Subjects wear a wrist actigraphy watch at night on the non-dominant wrist to monitor sleep quality and quantity.

### Example 4: Safety and Efficacy of Anti-Oncostatin M Receptor Beta Monoclonal Antibody in a First-In-Human Study

The study in this example is designed to evaluate the safety, tolerability, PK and immunogenicity of an anti-OSMRβ antibody in healthy subjects and in adult subjects with atopic dermatitis (AD) in a randomized, double-blind, placebo (PBO)-controlled, single-ascending dose study of the anti-OSMRβ antibody. AD was used as a proxy for IL-31-driven pruritic diseases to assess target engagement and Early Signal of Efficacy.

Study Design on Healthy Subjects and Subjects with Atopic Dermatitis

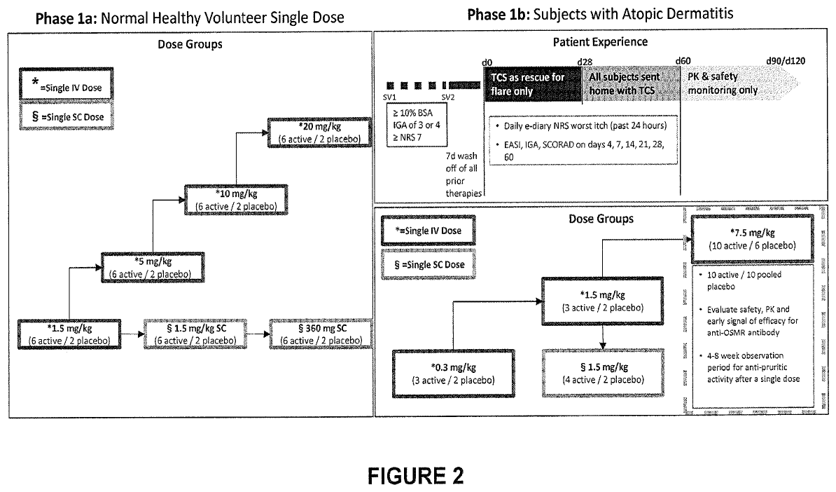

An anti-OSMRβ antibody was administered intravenously (IV) to four groups of adult healthy volunteer (HV) subjects. Additionally, the anti-OSMRβ antibody was administered subcutaneously (SC) to two groups of HV subjects. Three groups of AD subjects with moderate to severe atopic dermatitis experiencing moderate to severe pruritus were administered anti-OSMRβ antibody intravenously. Additionally, one group of AD subjects with moderate to severe atopic dermatitis experiencing moderate to severe pruritus was administered anti-OSMRβ antibody subcutaneously. The study design is outlined in FIG. 2.

### Dose Groups of Heathy Volunteer Subjects

HV subjects were enrolled into one of six groups as described below. After verification of eligibility, HV subjects were randomized to receive the anti-OSMRβ antibody or placebo. In four of the groups, the anti-OSMRβ antibody or placebo was administered IV. In the fifth and sixth groups, HV subjects received either the anti-OSMRβ antibody or placebo as a single SC injection.

The first group received 1.5 mg/kg anti-OSMRβ antibody or placebo intravenously; six HV subjects received the anti-OSMRβ antibody, and two HV subjects received placebo. The second group received 5 mg/kg anti-OSMRβ antibody or placebo intravenously; six HV subjects received the anti-OSMRβ antibody, and two HV subjects received placebo. The third group received 10 mg/kg anti-OSMRβ antibody or placebo intravenously; six HV subjects received the anti-OSMRβ antibody, and two HV subjects received placebo. The fourth group received 20 mg/kg anti-OSMRβ antibody or placebo intravenously; six HV subjects received the anti-OSMRβ antibody, and two HV subjects received placebo. The fifth group received 1.5 mg/kg anti-OSMRβ antibody or placebo subcutaneously; six HV subjects received the anti-OSMRβ antibody, and two HV subjects received placebo. The sixth group received 360 mg of anti-OSMRβ antibody or placebo subcutaneously; six HV subjects received the anti-OSMRβ antibody, and two HV subjects received placebo. The study design is represented graphically in FIG. 2, left panel.

Dose Groups of Subjects with Atopic Dermatitis

AD subjects were enrolled into one of four groups as described below. After verification of eligibility, AD subjects were randomized to receive the anti-OSMRβ antibody or placebo. In three of the groups, the anti-OSMRβ antibody or placebo was administered IV. In the fourth group, AD subjects received either the anti-OSMRβ antibody or placebo as a single SC injection.

The first group received 0.3 mg/kg anti-OSMRβ antibody or placebo intravenously; three AD subjects received the anti-OSMRβ antibody, and two AD subjects received placebo. The second group received 1.5 mg/kg anti-OSMRβ antibody or placebo intravenously; three AD subjects received the anti-OSMRβ antibody, and two AD subjects received placebo. The third group received 7.5 mg/kg anti-OSMRβ antibody or placebo intravenously; ten AD subjects received the anti-OSMRβ antibody, and six AD subjects received placebo. A fourth group of received 1.5 mg/kg anti-OSMRβ antibody or placebo via a subcutaneous dose; four AD subjects received the anti-OSMRβ antibody and two AD subjects received placebo. The study design is represented graphically in FIG. 2, lower right panel.

### Subject Inclusion Criteria

Adult subjects with moderate to severe AD experiencing moderate to severe pruritus were included; (Investigator Global Assessment [IGA] score of 3 or 4, body surface area [BSA]≥10%) experiencing moderate to severe pruritus; (worst itch Numerical Rating Scale [WI-NRS] at screening). Intravenous (IV) or subcutaneous (SC) anti-OSMRβ antibody was administered in escalating dose cohorts: HV IV: 1.5, 5, 10, and 20 mg/kg; HV SC: 1.5 mg/kg and 360 mg; AD IV: 0.3, 1.5 and 7.5 mg/kg; AD SC: 1.5 mg/kg (FIG. 2).

Safety and tolerability were assessed prior to dose escalation. Prohibited medications included topical corticosteroids (TCS) from Day-7 to Day 28; rescue medication was provided for AD flares. All subjects were given TCS to use as needed after Day 28.

Safety and tolerability data included vital signs, physical examination, ECG, laboratory measures, and adverse events (AEs). Anti-OSMRβ antibody target engagement and clinical pharmacodynamic (PD) data included daily e-diary WI-NRS and periodic Sleep-Loss Visual Analogue Scale (VAS) until Day 60. Weekly average of daily WI-NRS was calculated.

### Results

In total, 50 healthy volunteers (IV—24 active: 8 PBO; SC—12 active: 4 PBO) and 32 subjects with moderate-to-severe atopic dermatitis (AD) experiencing moderate-to-severe pruritus (IV—16 active: 10 PBO; SC—4 active: 2 PBO) received a single dose of anti-OSMRβ antibody or placebo in the Phase 1a/1b clinical trial, with the top dose of 20 mg/kg IV in healthy volunteers and 7.5 mg/kg IV in subjects with atopic dermatitis. There was a seven-day wash out period of prior therapies for all subjects with atopic dermatitis before treatment, and topical corticosteroids (TCS) were prohibited through Day 28. Rescue medication was provided for atopic dermatitis flares, and all subjects were given TCS to use as needed after Day 28. The anti-OSMRβ antibody was well-tolerated by all subjects, no dose-limiting toxicities were observed, and there were no serious adverse events.

Baseline demographics were balanced across dose groups, provided, however that the mean value of AD flares in the past year was higher in anti-OSMRβ antibody recipients than placebo recipients: 28.1 (SD=41.6) active vs. 3.7 (SD=3.5) PBO. No deaths, SAEs, or discontinuations due to AEs occurred. Drug-related treatment-emergent AEs were infrequent and showed no dose response correlation and all resolved without sequalae: in HVs, 1 mild headache (5 mg/kg IV), 1 mild flushing (1.5 mg/kg SC), and 1 mild anemia (360 mg SC); in AD subjects: 1 mild headache/mild decreased appetite (1.5 mg/kg IV), 1 moderate dizziness (7.5 mg/kg IV), 1 mild dizziness (1.5 mg/kg SC), and 1 mild somnolence (PBO IV). None of the following was observed in any patients treated with anti-OSMRβ antibody: deaths, Serious Adverse Events; discontinuations due to AEs; infusion reactions; injection site reactions; thrombocytopenia; peripheral edema; conjunctivitis.

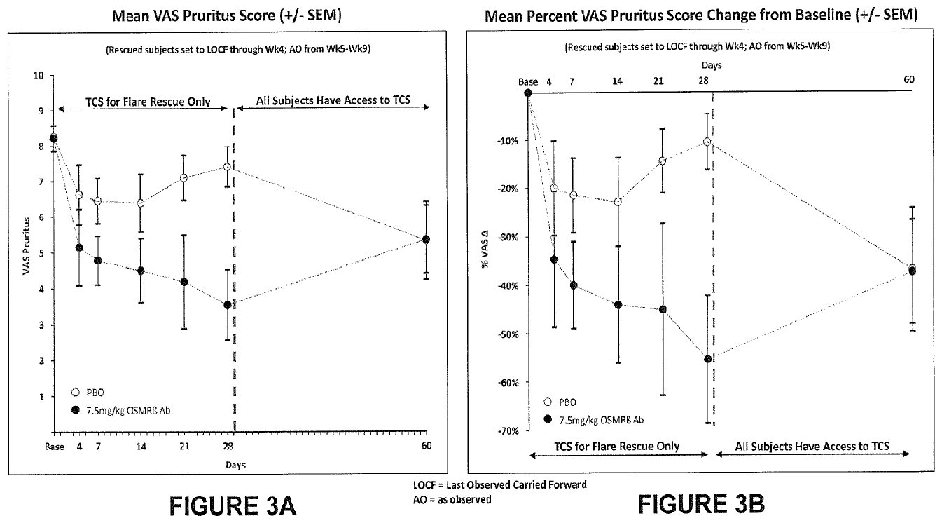

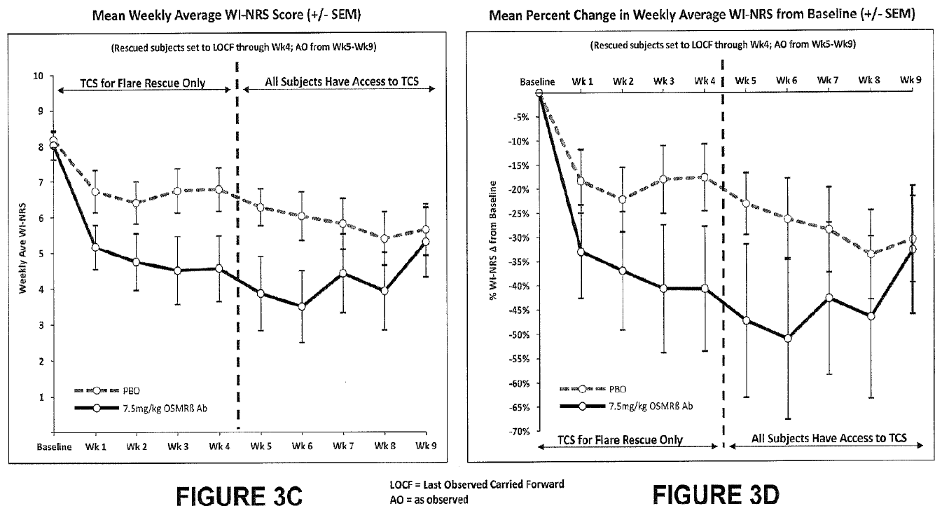

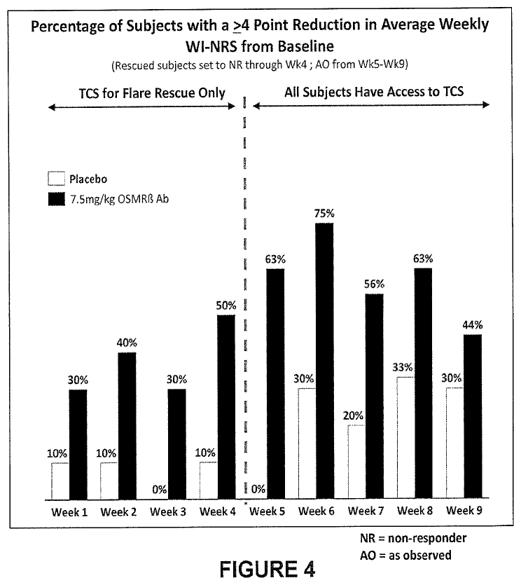

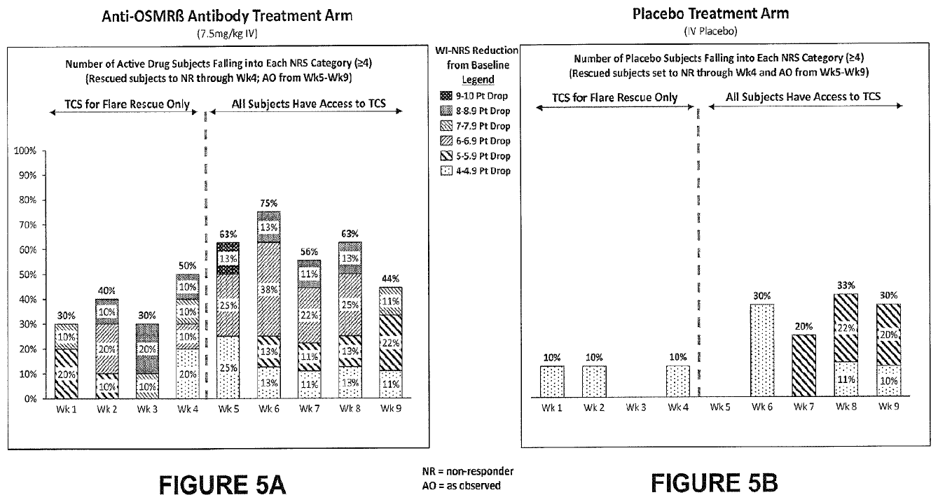

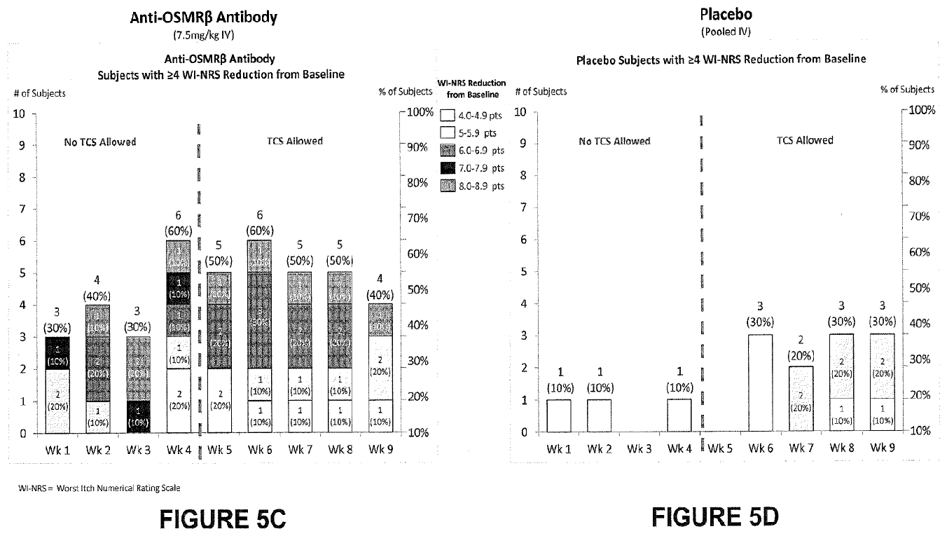

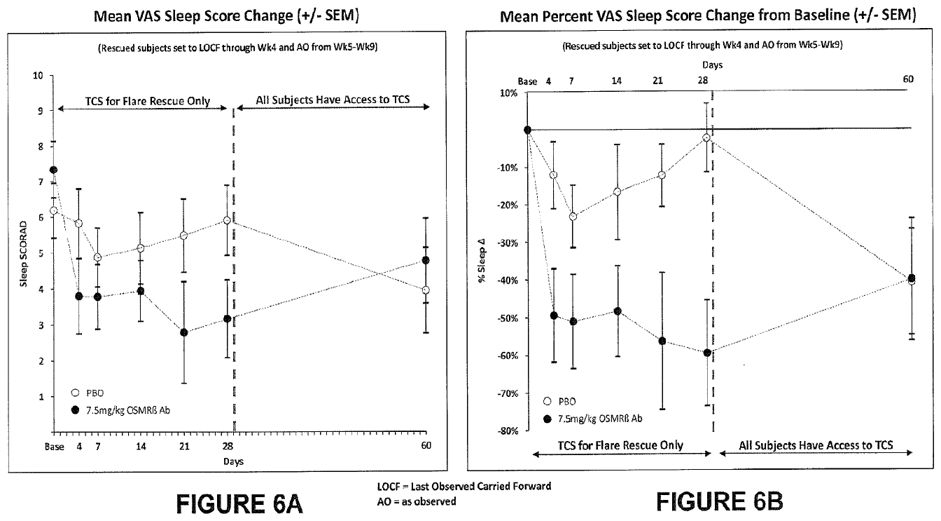

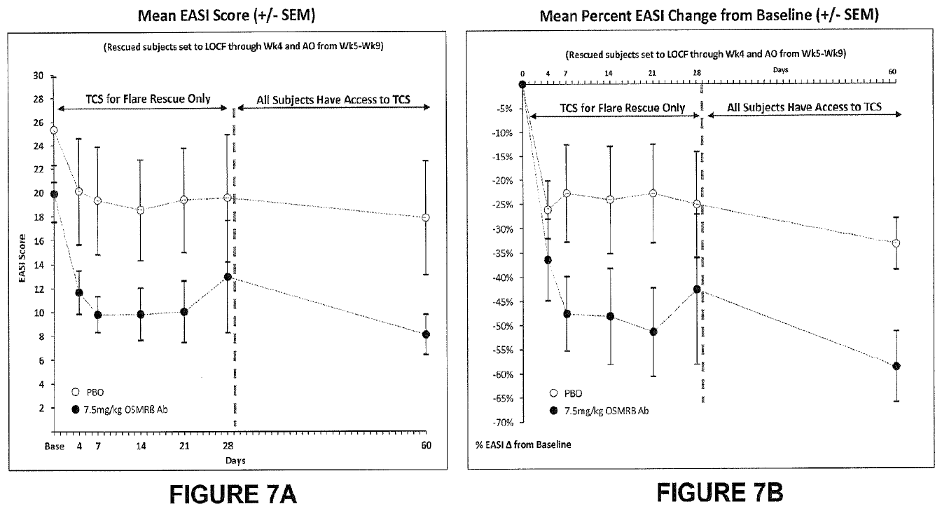

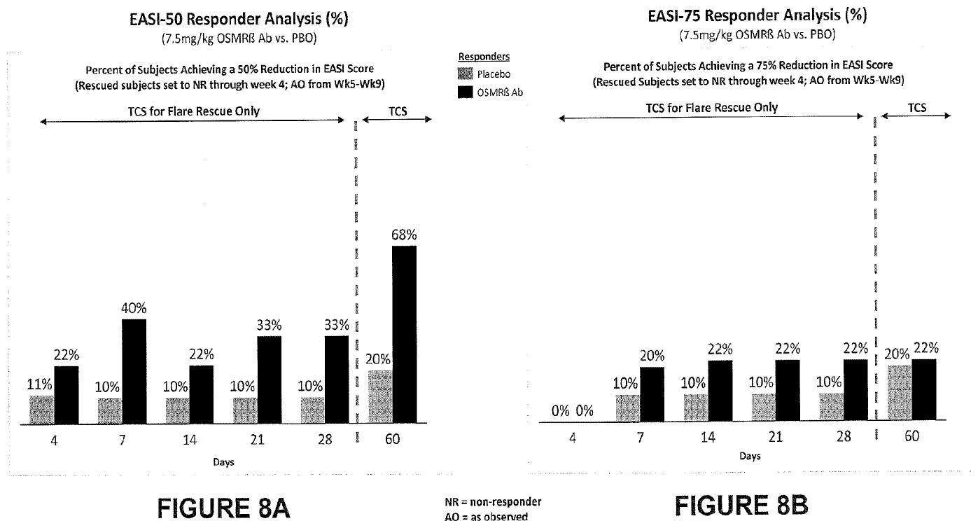

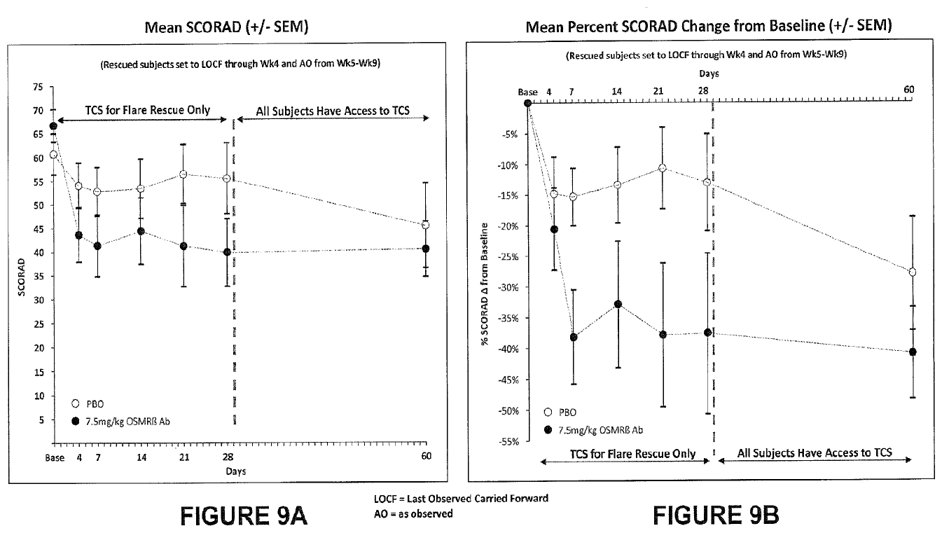

To assess target engagement and the clinical PD effect of anti-OSMRβ antibody in AD subjects after a single dose, the weekly average pruritus WI-NRS on Day 28 was compared between anti-OSMRβ antibody recipients at 7.5 mg/kg IV (n=10) and pooled PBO IV recipients (n=10). Baseline mean weekly average pruritus NRS was balanced: 8.0 (anti-OSMRβ antibody) vs. 8.2 (PBO); between Day 0 to Day 60, AD flares occurred in 3 anti-OSMRβ antibody recipients and 3 PBO recipients. One recipient of 0.3 mg/kg IV anti-OSMRβ antibody had a flare on day 7; two recipients of 7.5 mg/kg IV anti-OSMRβ antibody had flare on days 14, day 20 respectively; three PBO recipients had flares on day 1, day 5, and day 45 respectively. At the point of a flare these patients were provided with topical cortical steroids (TCS) as a rescue therapy. In FIGS. 3A-8B, a “Last Observation Carried Forward” (LOCF) or “Non-Responder” (NR) approach was applied to data values for Weeks 1-4. However, for the extended period after Week 4 up to Week 9, when all patients had access to TCS, the “As Observed” (AO) statistical approach was applied to the data. A dotted line through the figures indicate the two phases. Mean change from baseline in weekly average Pruritus Visual Analog Scale (VAS) are shown in FIG. 3A. FIG. 3B shows mean percent change in VAS pruritus score from baseline. Worst Itch Numerical Rating Scale (WI-NRS) are shown in FIG. 3C. Mean change in weekly average WI-NRS from baseline is shown in FIG. 3D. Mean percentage change in weekly average pruritus VAS (a component of SCORAD) was greater in anti-OSMRβ antibody recipients vs. PBO: −55.4% active vs. −10.4% PBO on Day 28 (FIG. 3B). Mean percentage change in weekly average WI-NRS was greater in anti-OSMRβ antibody recipients vs. PBO: −40.7% active vs. −17.6% PBO on Day 28 (FIG. 3D). FIGS. 4 and 5A-5D show the percentage of subjects with a ≥4-point reduction in average weekly WI-NRS from baseline. A ≥4 point reduction in NRS from baseline is generally considered a clinically meaningful change. A higher percentage of anti-OSMRβ antibody recipients demonstrated a ≥4-point decrease in weekly average WI-NRS vs. PBO consistently throughout the duration of the study as shown in FIG. 4. At week 4, 50% of the active group demonstrated a ≥4-point decrease in weekly average WI-NRS vs. 10% in the PBO group. FIG. 5A-5D shows the percentage of subjects who responded with a particular magnitude of NRS reduction from baseline (≥4 points). FIGS. 5A and 5C show the respective percentages of anti-OSMRβ antibody recipients and FIGS. 5B and 5D show the respective percentages of placebo recipients. In FIGS. 4, 5A and 5B, responder rates were calculated using a denominator that includes subjects with non-missing values. In FIGS. 5C and 5D responder rates were calculated using a denominator that includes all subjects. Rescued subjects were considered non-responders in this assessment. The anti-OSMRβ antibody recipients demonstrated a greater magnitude decrease in weekly average WI-NRS vs. PBO consistently throughout the duration of the study. The maximum decrease in WI-NRS at day 28 was greater in anti-OSMRβ antibody recipients vs. PBO: ≥8 points active vs. 4 points PBO. Further, at week 3, a ≥7-point decrease was observed in 30% of anti-OSMRβ antibody recipients vs 0% in the placebo group. The overall maximum decrease in WI-NRS observed during the study period following the initial 28 days (i.e., with the use of concomitant TCS) was greater in anti-OSMRβ antibody recipients vs. PBO: ≥9 points active vs. 5-5.9 points PBO. ≥9 point decrease was observed in 13% of the anti-OSMRβ antibody recipients (FIG. 5A), vs 0% of the placebo group on week 5 (FIG. 5B). The anti-OSMRβ antibody recipients demonstrated a persistent effect on weekly-average WI-NRS through Day 56 in combination with the use of concomitant TCS during the adjunctive therapy period (FIG. 3C-D, FIG. 4, and FIGS. 5A-D). Concordant with the effect on pruritus, anti-OSMRβ antibody recipients reported improved sleep vs. PBO (FIGS. 6A-B), as evidenced by a greater decrease in sleep-loss VAS (a component of SCORAD): −59.5% active vs. −2.3% PBO on Day 28 (FIG. 6B). FIG. 7A-B shows the change in Eczema Area and Severity Index (EASI) from baseline in antibody and placebo recipients. Mean percentage change in Eczema Area and Severity Index (EASI) from baseline is shown in FIG. 7B. The reduction in EASI was greater in anti-OSMRβ antibody recipients vs. PBO: −42.59% vs. −25.07% at week 4, as shown in FIG. 7B. The anti-OSMRβ antibody demonstrated a reduction in disease severity as determined by subjects achieving a 50% or greater reduction in EASI score (EASI-50), shown in FIG. 8A. On Day 28, 44% of anti-OSMRβ antibody recipients achieved EASI-50, compared to 20% of the placebo group. Additionally, as shown in FIG. 8B, 27% of the anti-OSMRβ antibody recipients achieved EASI-75, indicating 75% reduction in EASI, compared to 10% of the subjects in the placebo group on Day 28. FIGS. 9A and 9B depict the mean SCORAD values and mean percent SCORAD changes from baseline respectively, shown through a period up to Day 60. These data demonstrate the safety and tolerability profile, pharmacodynamic effect and impact on quality of life of OSMRβ inhibition in AD patients. A single dose of OSMRβ antibody at 7.5 mg/kg resulted in serum levels above 5 μg/mL (5.8-28.2 μg/mL) in 80% of recipients 44 to 47 days post-dose. In addition, using the as-observed dataset, WI-NRS, pruritus VAS, and sleep-loss VAS were compared between 10 anti-OSMRβ antibody (7.5 mg/kg IV) recipients and 10 PBO IV recipients between days 29-60. Anti-OSMRβ antibody recipients experienced a greater WI-NRS improvement that continued into the adjunctive therapy period during which they received concomitant TCS and reached a maximum level at 6 weeks: −51% vs −26.3%. A higher percentage of anti-OSMRβ antibody recipients demonstrated a ≥4-point decrease in WI-NRS vs PBO, reaching a maximum differential in the adjunctive therapy period during which they received concomitant TCS at 5 weeks: 63% vs 0%. Differences between anti-OSMRβ antibody and PBO recipients in improvement in pruritus or sleep loss VAS also extended into the adjunctive therapy period.

Subcutaneous vs. Intravenous Administration

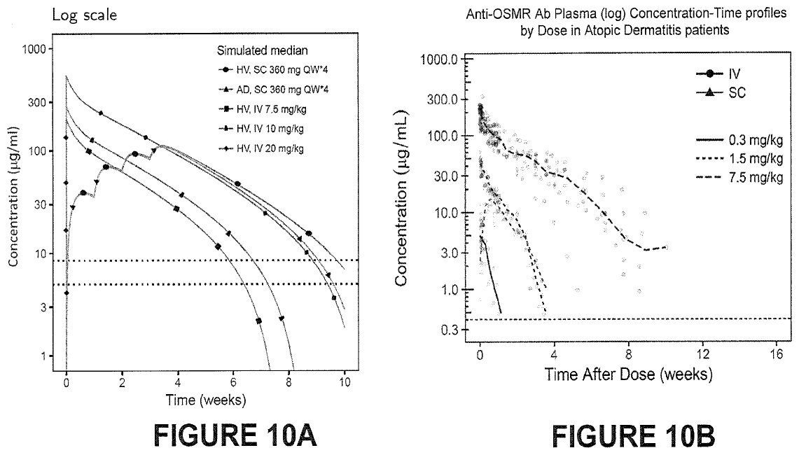

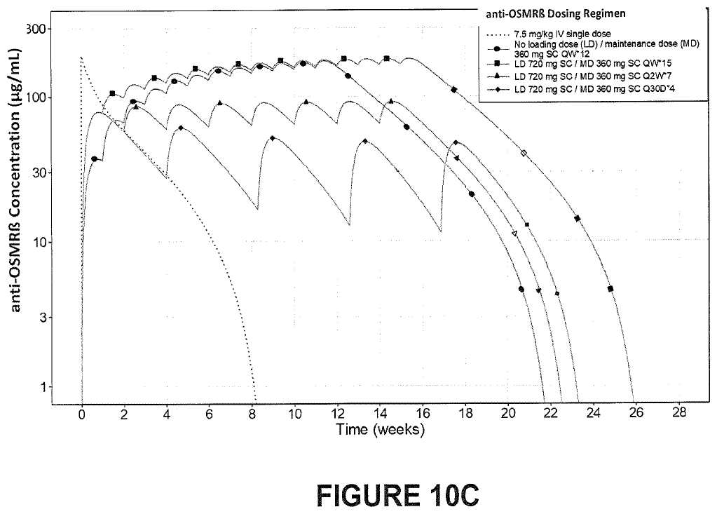

PK parameters were modeled to assess the viability of subcutaneous administration. The simulated plot in FIG. 10A was derived from plasma concentrations of anti-OSMRβ antibody in non-human primates, HV and AD patients. FIG. 10A shows a simulated median plot of antibody concentration in plasma over the indicated time in weeks following subcutaneous (SC) or intravenous (IV) administration to heathy volunteers (HV) or Atopic Dermatitis (AD) patients. The upper dotted line indicates the EC₉₀ of the anti-OSMRβ antibody in providing protection from supra-physiologic human IL-31 challenge-induced pruritus in non-human primates. The lower dotted line indicates the EC₇₅ of the anti-OSMRβ antibody in providing protection from supra-physiologic human IL-31 challenge-induced pruritus in non-human primates. EC₇₅ and EC₉₀ were determined from the study described in Example 1. FIG. 10B shows plasma anti-OSMRβ antibody concentration profiles for the indicated doses in Atopic Dermatitis (AD) patients. Following single dose IV and SC administration, anti-OSMRβ antibody exposure (as measured by AUC0-∞) was similar in healthy volunteers and Atopic Dermatitis patients and approached linearity with increasing dose levels. Bioavailability between healthy volunteers and AD subjects at the evaluated SC dose levels was generally comparable (42% vs. 65%, respectively). Anti-OSMRβ antibody showed dose-dependent elimination consistent with a target-mediated drug disposition (TMDD) profile. At the 7.5 mg/kg IV dose level, anti-OSMRβ antibody was detectable through at least 8 weeks. The modeled PK parameters predict viability of subcutaneous administration, and predict that a fixed subcutaneous dose of 360 mg of anti-OSMRβ antibody can achieve exposures similar to a 7.5 mg/kg IV dose of the antibody.

A separate modeling simulation was carried out to characterize the PK of anti-OSMRβ antibody following IV and SC administration in adult healthy volunteers (HV) and subjects with AD, and investigate various SC dosing regimens to optimize practical chronic dosing in a target population. Single dose data from a Phase 1b clinical study in 57 HV and subjects with AD were analyzed. Most HV and AD subjects received weight-based IV administration (n=24, n=16, respectively; range: 0.3-20 mg/kg), followed by weight-based SC (n=6, n=4; 1.5 mg/kg) and fixed-dose SC (HV, n=7, 360 mg) administration. The PK of the anti-OSMRβ antibody in HV and AD subjects following single-dose IV or SC administration was described using a target-mediated drug disposition (TMDD) model to account for its non-linear clearance. Association and dissociation rate constants were determined experimentally at 0.734 nM·hr⁻¹ and 0.268 nM·hr⁻¹, respectively, and fixed during model development. Relative bioavailability of SC administration in AD was estimated for the model at 65% (based on the comparison of PK of 1.5 mg/kg IV and SC in HV and AD subjects and then revised for dose-dependency based on PK of 360 mg SC in HVs). Body weight was included as a covariate on the central volume of distribution based on allometric theory. FIG. 10C depicts simulations of various dosing regimens using the final population PK model. A range of simulations performed to evaluate various SC dosing regimens using an exemplary dose of anti-OSMRβ antibody (360 mg in 2 mL SC injection). Exposure metrics and time to steady-state were derived for each simulated SC dosing regimen. The model (including TMDD) was used to simulate future dosing scenarios for chronic SC dose administration in patients with chronic pruritic diseases in which the target receptor may be upregulated. This model also supports determination of practical chronic dose(s)/dosing intervals using a Car derived from clinical trials with anti-OSMRβ antibodies of the invention.

### Example 5. Pathogenesis of Prurigo Nodularis and Role of IL-31

The study in this example characterized prurigo nodularis pathogenesis and evaluated the role of IL-31 in the mechanism of the disease. Prurigo nodularis (PN) is a chronic skin disease of unknown etiology characterized by symmetrically-distributed, intensely-pruritic hyperkeratotic nodules. Comorbidities featuring chronic pruritus are implicated in PN pathogenesis by initiating the itch-scratch cycle that leads to nodule formation. The role of OSMRP, the shared receptor subunit for IL-31 and oncostatin M (OSM) signaling, involved in pruritus, inflammation and fibrosis, in PN pathogenesis is unknown. The role of IL-31 in the disease mechanism of PN is herein elucidated.

### Study Design and Method

A prospective longitudinal/observational study was conducted in US and Europe which investigated PN pathophysiology. Medical history, pruritus (eDiary), sleep, quality of life, disease severity, blood, and skin biopsies were collected at baseline and up to 12 months. Skin biomarker gene expression (RT-PCR; RNA) and immunohistochemistry (IHC; protein) results were correlated with worst itch Numerical Rating Scale (WI-NRS). Gene expression results were benchmarked to atopic dermatitis (AD) and normal skin.

### Results

54 patients were enrolled. Most (n=35, 65%) had PN with no underlying comorbidities identified: 4 had no other medical condition; 31 had other medical conditions, but none was considered causally-related. 19 (35%) had other medical conditions that were considered causally-related.

Pruritus intensity (eDiary worst-itch NRS), sleep impairment, and disease severity (number of lesions and percent with excoriation/crust) were similar regardless of the presence or absence of underlying comorbidities. Furthermore, IL-31-expressing mononuclear cells were present in 89% of lesional biopsies (immunohistochemistry) whether or not an underlying condition was identified. IL-31, IL-31Rα, OSM, and OSMRβ expression in mononuclear cells were upregulated in lesional biopsies versus non-lesional biopsies (p≤0.001). IL-31 mRNA was expressed in 44% of lesional PN, 16% of non-lesional PN, 12.5% of healthy volunteer, and 100% of AD biopsies (lesional [LS] and non-lesional [NL]). IL-31 mRNA was expressed in 64% of LS biopsies from PN patients with WI-NRS≥7. IL-31 protein (IHC) was expressed in mononuclear cells in the majority of LS PN biopsies (89%) vs 44% of NL PN biopsies. Polymorphonuclear cells (PMNs), when present, and endothelial cells were other common sources of IL-31 in LS PN skin. Expression of OSM, IL-31Rα, and OSMRβ mRNA was ubiquitous (74-100%) in LS or NL PN, AD or healthy volunteer biopsies. However, a higher proportion of LS PN biopsies contained mononuclear cells expressing IL-31Rα (1.7-fold), OSM (3.6-fold), and OSMRβ (1.8-fold) protein than NL PN biopsies. Epidermal cells, and when present, PMN, dermal nerves, and adnexal structures were other common sources of IL-31Rα and OSMRβ in LS PN skin. See Example 7 for further details of this study.

The data indicated that PN is a distinct, highly pruritic chronic skin disease that is not defined by its comorbid conditions. IL-31 is implicated in the pathogenesis of PN given its prevalent expression in PN nodules. Thus, the role of IL-31 in the disease mechanism of PN is hereby elucidated.

### Example 6. Effect of Anti-OSMR-β Antibody on Th2 Signaling Pathway in Keratinocytes

The study in this example further demonstrated that the anti-OSMRβ antibody of the invention can effectively treat inflammation. The objectives of these studies were to characterize the in vitro responses of human epidermal keratinocytes (HEK) and human dermal fibroblasts (HDF) to OSM in comparison to LIF and IL-31, using chemokine monocyte chemoattractant protein 1 (MCP-1/CCL-2), which has roles in inflammatory responses. An additional objective of the studies presented in this example was to assess the ability of the anti-OSMRβ antibody of the invention to effectuate MCP-1/CCL2 responses in HEK and HDF cells.

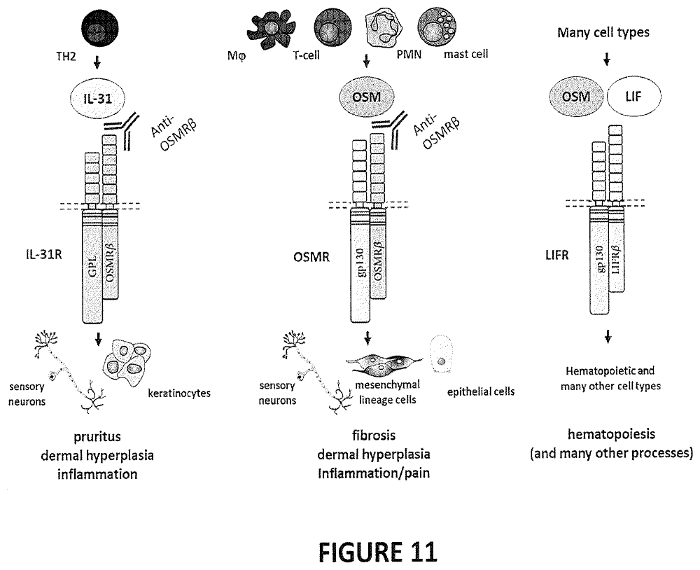

FIG. 11 shows the receptor structure for IL-31 signaling and that of OSM signaling. OSM interacts with two receptors in humans, a type I receptor and a type II receptor. The type I receptor complex comprises a receptor heterodimer of LIFRα and gp130. The type II receptor complex comprises a receptor heterodimer of OSMRβ and gp130. In particular, the data presented in this example show that administering an anti-OSMRβ antibody targets and attenuates OSM-mediated T_{H}2 inflammatory signaling pathway in human epidermal keratinocytes (HEK) and human dermal fibroblasts (HDF) cells. The data also indicate that the antibody can inhibit OSM-mediated inflammatory pathways independent of IL-31 involvement.

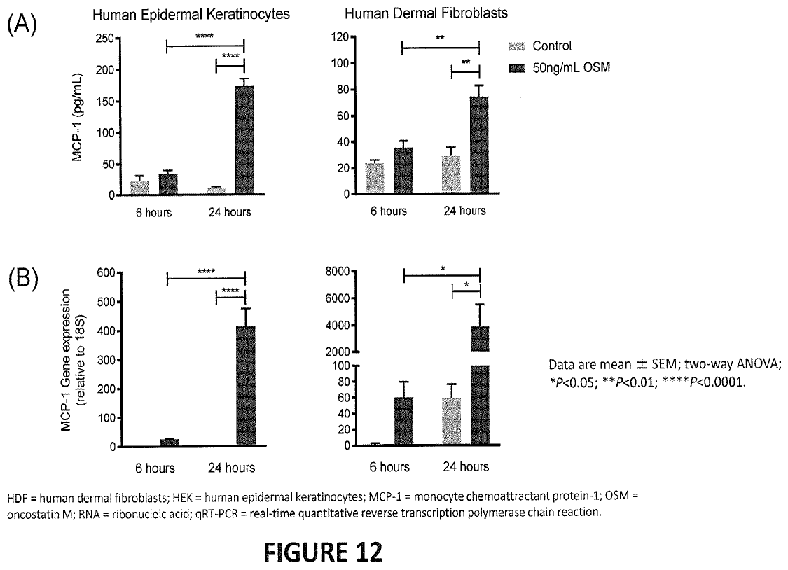

Oncostatin M (OSM), a member of the gp130 cytokine family, is involved in T_{H}2 inflammation, epidermal integrity, and fibrosis. The effect of OSM on monocyte chemoattractant protein 1 (MCP-1, also known as “CCL2”) was evaluated with and without interleukin (IL)-4, IL-13, and the anti-OSM receptor f3 (OSMRβ) monoclonal antibody of the invention in human epidermal keratinocytes (HEK) and human dermal fibroblasts (HDF) in vitro. Cells were stimulated with OSM, leukemia inhibitory factor (LIF), IL-31, IL-13, alone or in combination, and separately with OSM, OSM+IL-4, and increasing concentrations of the anti-OSMRβ monoclonal antibody. MCP-1 levels in supernatants were determined by ELISA. MCP-1 and receptor chain mRNAs were measured. OSM (50 ng/mL) strongly induced MCP-1 protein (in HEK; p<0.0001 and HDF; p<0.01, FIG. 12, panel A) and mRNA (in HEK; p<0.0001 and HDF; p<0.05, FIG. 12, panel B) at 24 hours In HEK, OSM (but not LIF or IL-31) induced phosphorylation of STAT3 or STAT1 and synergized with either IL-13 or IL-4 in elevating MCP-1 (p<0.01). Results were similar for OSM in HDF; LIF or IL-31 minimally activated STAT3 but not MCP-1.

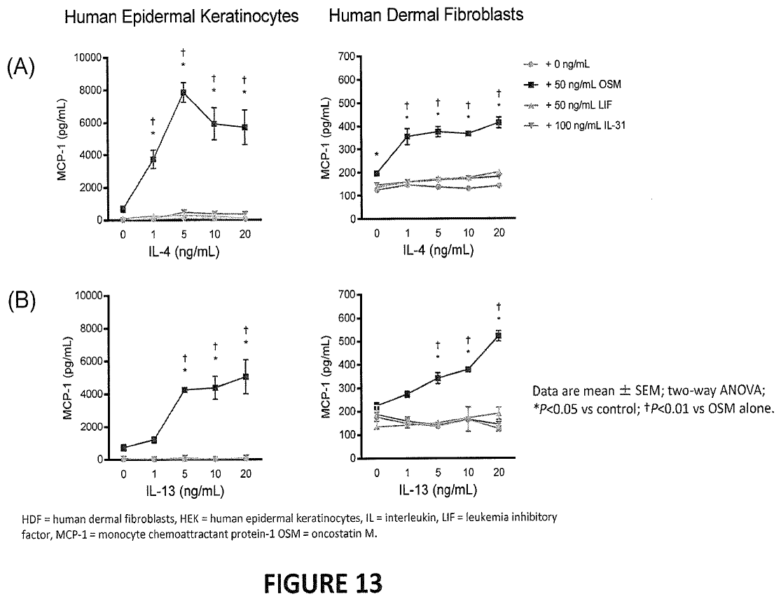

A dose-dependent increase in MCP-1 production was observed for IL-4 or IL-13 in combination with OSM (p<0.01) in both HEK and HDF cells (FIG. 13). These results showed that OSM synergizes with IL-4 or IL-13 in the induction of MCP1/CCL-2 in HEK and HDF. Notably, the dose-dependent increase in MCP-1 production was not observed for IL-4 or IL-13 in combination with LIF or IL-31. Furthermore, IL-4 or IL-13 alone did not induce MCP-1/CCL-2 levels at any concentration assessed.

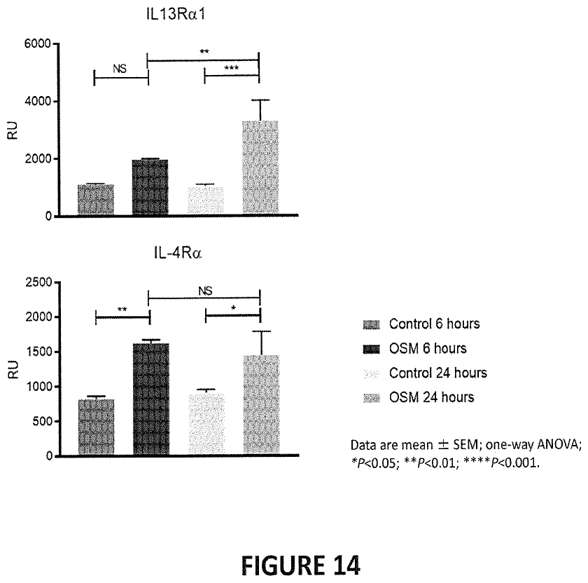

In HEK and HDF cells, OSM significantly induced mRNA for the receptor chains of type II IL-4 receptor (IL-4Rα/IL13Rα) and type II OSM receptor OSMRβ/gp130 (HEK, p<0.05; HDF, p<0.01;), but not for chains of LIF receptor or IL-31Rα. The data in FIG. 14 were obtained from HEK cells and show an increase in IL13Rα and IL-4Rα mRNA at 6 hours and 24 hours after treatment with OSM. These data indicate that OSM stimulates mRNA for the receptor chains of type II IL-4 receptor and type II OSM receptor complexes in HDF cells.

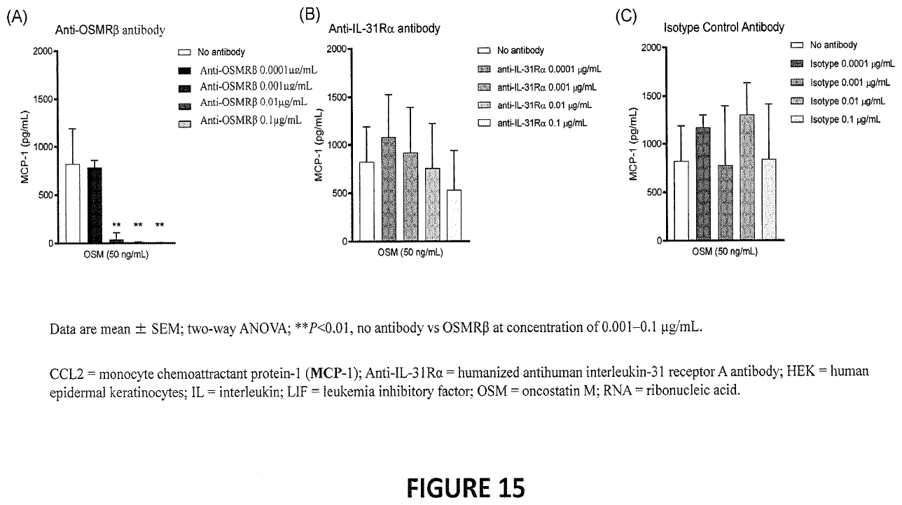

Studies were also performed to test the effect of adding the anti-OSMRβ antibody of the invention to cell cultures that had been induced with OSM in HEK cells. For these studies, HEK were cultured at 15,000 cells/well in 96-well plates and stimulated with OSM (50 ng/mL) for 24 hours (n=4/treatment) in the presence of increasing concentrations (as indicated) of either anti-OSMRβ, anti-IL31Rα, or control isotype antibody. Culture supernatants were removed and stored at −80° C. before analysis by ELISA for MCP-1. The results of these studies indicated that anti-OSMRβ antibody inhibits OSM-induced MCP-1/CCL-2 in HEK cells (FIG. 15, panel A). Specifically, at concentrations of 0.001 μg/mL and higher of anti-OSMRβ, MCP-1/CCL-2 levels were markedly reduced.

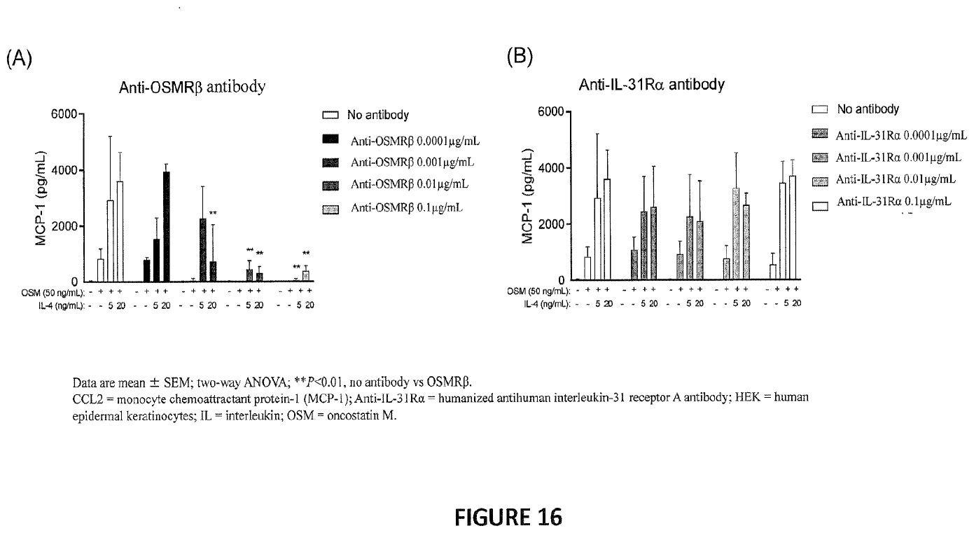

Additional studies were performed to assess whether anti-OSMRβ reduced MCP-1/CCL-2 levels associated with the synergistic response to OSM and IL-4. For these studies, HEK were cultured at 15,000 cells/well in 96-well plates and stimulated with OSM (50 ng/mL) and IL-4 at either 5 or 20 ng/mL concentrations for 24 hours (n=4/treatment) in the presence of increasing concentrations (as indicated) of either (A) anti-OSMRβ or (B) anti-IL31Rα. Culture supernatants were removed and stored at −80° C. before analysis by ELISA for MCP-1. The data showed that anti-OSMRβ reduced MCP-1/CCL-2 levels associated with the synergistic response to OSM and IL-4 at both concentrations tested (FIG. 16, panel A).

Anti-IL-31Rα or isotype control antibody had no significant effect on the OSM− and OSM+IL-4-induced responses (FIG. 15, panels B and C, and FIG. 16, panel B). Collectively, the data presented in this example show that OSM regulates expression of pro-inflammatory chemokine MCP-1/CCL-2 in HEK and HDF cells. These data also show that OSM synergizes with T_{H}2 cytokines (IL-4 and IL-13) to induce MCP-1/CCL-2 in the cells, while LIF or IL-31 do not in this system. These data suggest a separate pathway for OSM signaling in HEK and HDF cells. The anti-OSMRβ monoclonal antibody reduced both the OSM induction and the synergistic OSM+IL-4 induction of MCP-1/CCL-2 protein production. Potent inhibition of OSM activity suggests therapeutic potential of the anti-OSMRβ monoclonal antibody in T_{H}2-mediated diseases distinct from the anti-OSMRβ antibody's inhibition of IL-31.

Similar studies can be extended to assess the effect of the anti-OSMRβ antibody of the invention on IL-6, IL-8 and other mediators of inflammatory pathway.

### Example 7: Prurigo Nodularis Biomarker Analysis and Clinical Endpoints

This example shows the results of an investigation into IL-31, IL-31Rα, OSM and OSMRβ RNA and protein expression levels in skin biopsies of prurigo nodularis (PN) patients as compared with healthy skin biopsy samples. The data obtained from the studies described herein showed that OSMRβ axis molecules IL-31, OSM, IL-31Rα, and OSMRβ are present in PN and in atopic dermatitis (AD) skin samples. In PN patients, IL-31 was detected more frequently in lesional (LS) biopsies than in non-lesional (NL) biopsies. Furthermore, the intensity or upregulation of IL-31 expression increased with itch severity in PN patients.

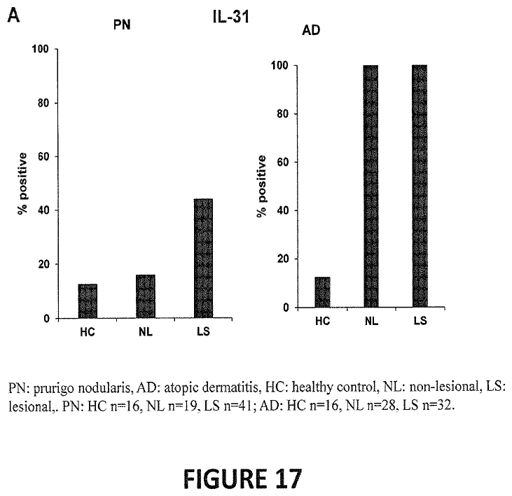

Gene expression measurements of NL and LS skin biopsies were performed to assess IL-31 mRNA expression levels in comparison to healthy control subjects. The data from these studies showed that there was a marked increase in the mRNA levels found in PN and AD biopsies in comparison to heathy controls. IL-31 mRNA expression was detected in 44% of LS PN, 16% of NL PN, 12.5% of healthy volunteer and 100% of AD (LS and NL) biopsies (FIG. 17).

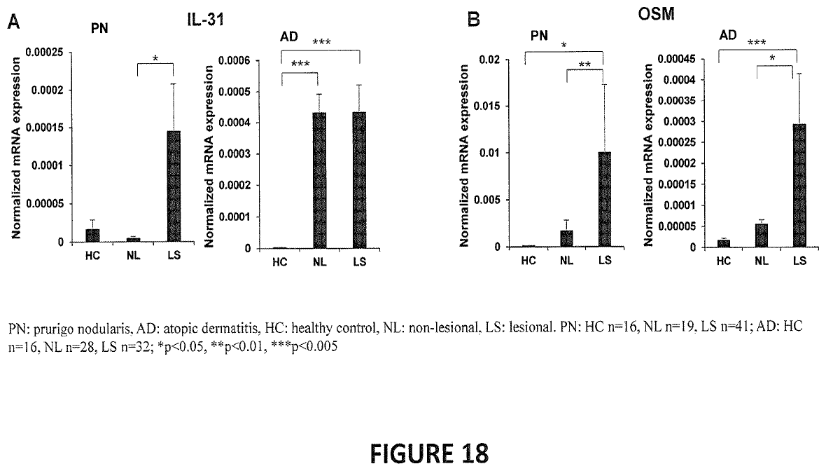

Additional studies were performed with PN and AD skin biopsies to determine the levels of IL-31 and OSM expression in comparison to healthy skin (FIG. 18, panels A and B). The data showed that cells from LS PN biopsies expressed significantly higher levels of IL-31 and OSM compared to NL or healthy biopsies.

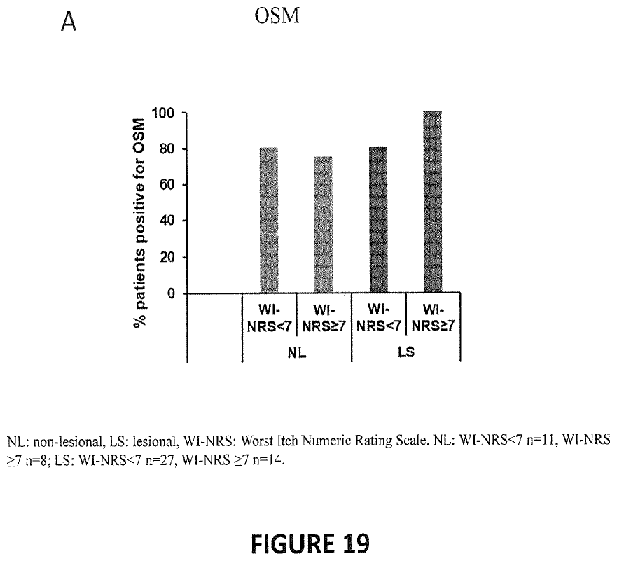

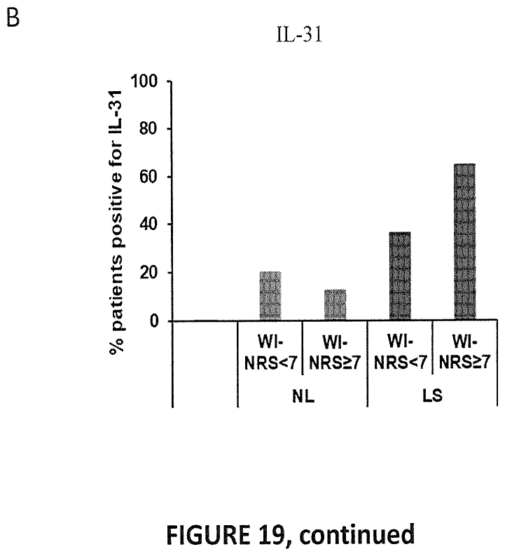

Skin biopsies obtained from patients who have WI-NRS itch scores of different scales showed that IL-31 and OSM levels correlate with itch scores. For these studies, patients were grouped based on baseline WI-NRS score. FIG. 19, panel A shows that OSM expression levels were increased in NL and LS biopsies of patients who have WI-NRS≥7. IL-31 expression was also increased in patients who have WI-NRS≥7 (FIG. 19, panel B).

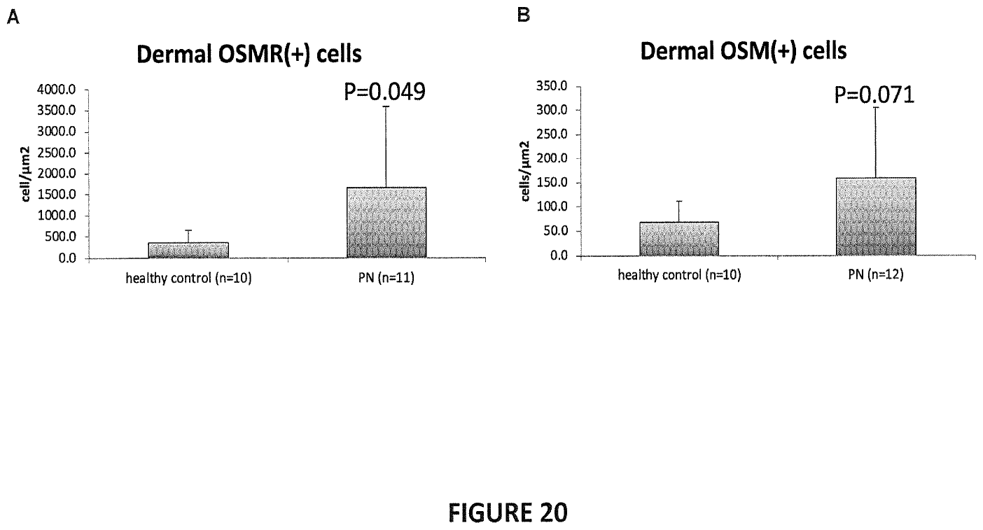

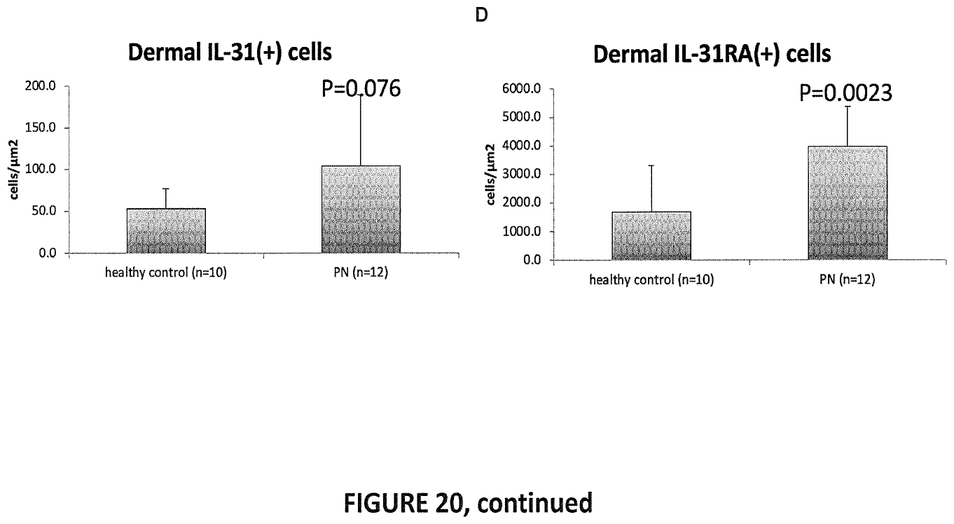

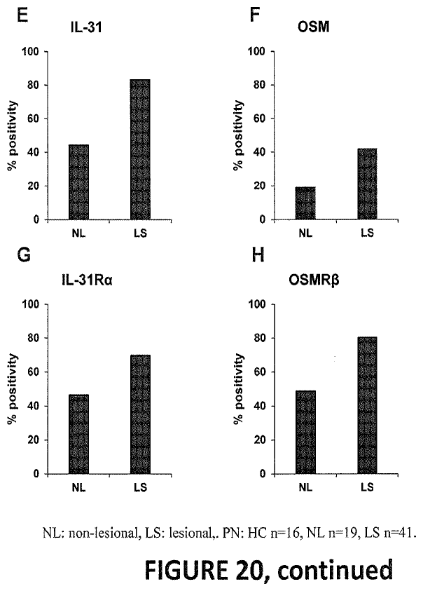

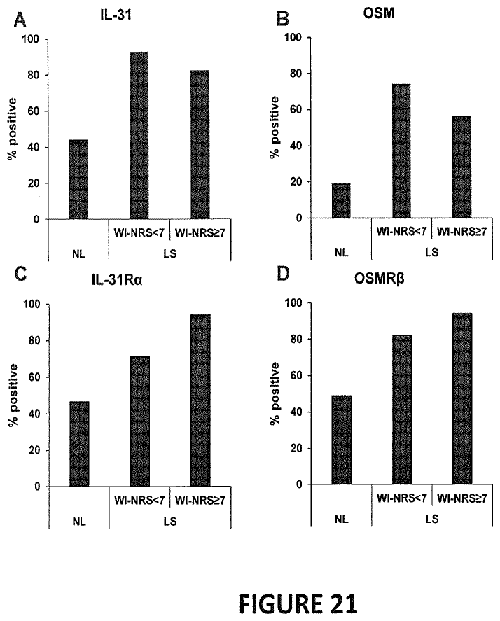

Additional immunohistochemistry studies were performed on sectioned biopsy samples. For these studies, skin samples obtained from subjects who have prurigo nodularis (PN) were sectioned and processed for immunohistochemical analysis with antibodies specific for OSMRβ, OSM, IL-31 or IL-31Rα. Positive cell counts were measured in the epidermal junction using ImagJ software. The results from these studies showed that subjects who have PN have an increased number of cells in the dermis that are positive for OSMRβ, OSM, IL-31 and IL-31Rα in comparison to healthy control subjects (FIG. 20, panels A-D). Further immunohistochemistry analysis was performed on biopsies obtained from PN subjects and the results showed that lympho-monocytes and endothelial cells are common sources of IL-31 and OSM in both NL and LS tissues. Furthermore, lympho-monocytes from LS biopsies showed significantly higher expression of all target proteins, compared to NL biopsies, (FIG. 20, panels E-H). These studies further showed that IL-31Rα and OSMRβ protein levels in lympho-monocytes correlated with itch severity (FIG. 21, panels A-D).

Collectively, the data presented in this example shows the prevalent expression of the OSMRβ axis (IL-31, OSM, IL31Rα, and OSMRβ) in PN and AD lesional skin. These data suggest that expression of these factors play a role in the pathogenesis of skin inflammatory conditions. These data indicate that targeting OSMRβ using an antibody as described herein is useful for the treatment of PN.

### Example 8: OSMRβ mRNA and Protein Levels are Increased in Urticaria Skin Biopsies

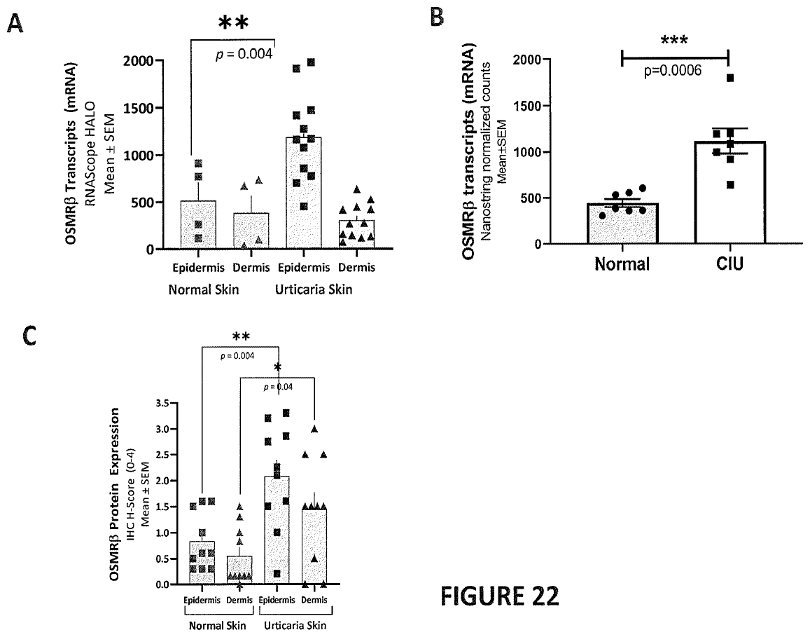

Skin biopsy samples were obtained from subjects who have chronic idiopathic urticaria (CIU) and from control subjects who do not have an inflammatory or pruritic skin disease or disorder in order to assess OSMRβ mRNA and protein expression levels in the samples. For these studies, OSMRβ mRNA levels were assessed using RNAscope® in situ hybridization (ISH) and nanoString® technologies (FIG. 22, panels A and B, respectively). For the RNAscope® ISH studies, 12 human CIU skin samples and 4 human normal skin samples were evaluated in in accordance with standard methods. The same patient samples were used to evaluate OSMRβ mRNA expression by RNAscope® and nanoString® technologies. To assess the OSMRβ protein expression, immunohistochemistry (IHC) was performed on sections obtained from separate skin biopsies. An IHC H-score was determined for each of the samples analyzed and the results were plotted on a graph (FIG. 22, panel C).

These studies showed that OSMRβ mRNA and protein expression levels were significantly increased in CIU skin in comparison to skin obtained from subjects who do not an inflammatory or pruritic skin disease or disorder (FIG. 22, panels A-C). There was high consistency between the OSMRβ mRNA results as assessed by RNAscope® and by Nanostring® technologies, each of which showed a significant (p=0.004 and p=0.002, respectively) upregulation in the levels of OSMRβ mRNA transcripts in the urticaria subject sample in comparison to the control sample (“normal skin”). Furthermore, the data obtained from the IHC studies showed a significant elevation in protein expression levels in both the epidermis and the dermis of subjects who have CIU in comparison to the control sample (FIG. 22, panel C).

Collectively, the data obtained from these studies showed that OSMRβ mRNA and protein levels are increased in the skin of subjects who have CIU in comparison to subjects who do not have an inflammatory or pruritic skin disease or disorder. These data indicate that targeting OSMRβ using an antibody as described herein is useful for the treatment of CIU.

### Example 9: OSMRβ mRNA is Increased in Lichen Simplex Chronicus (LSC), Lichen Planus (LP) Skin Biopsies and Chronic Idiopathic Pruritus (CIP)

Skin biopsy samples were obtained from subjects who have Lichen Simplex Chronicus (LSC) and Lichen Planus (LP). The skin samples were analyzed for mRNA expression of OSMRβ using standard NanoString® or RNAscope® technology. As a control for these studies, skin biopsies were also obtained from subjects who do not have an inflammatory or pruritic skin disease or disorder.

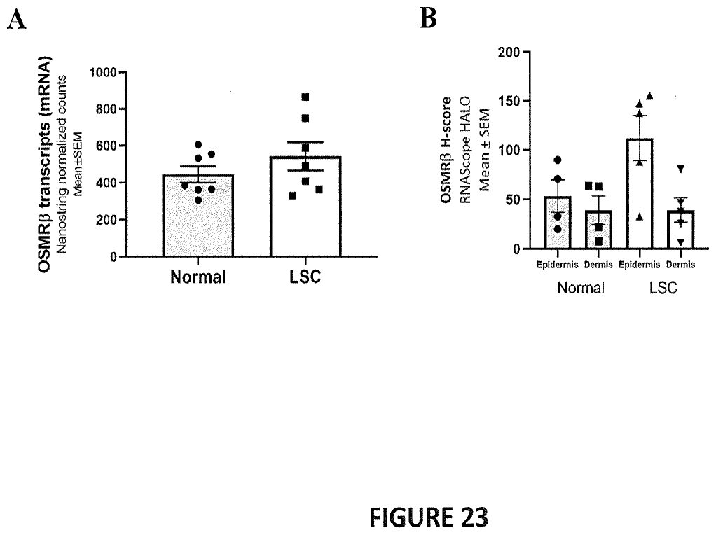

The data obtained from these studies showed that OSMRβ mRNA levels are increased in subjects who have LSC (FIG. 23, panels A and B). The data were obtained using either NanoString (panel A) or RNAScope (panel B) methodology.

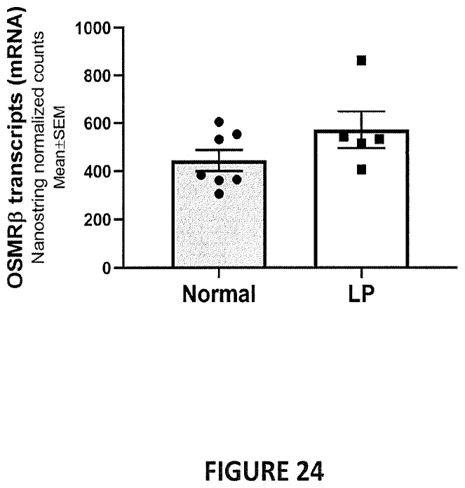

Data were also obtained from skin samples isolated from subject who have LP (FIG. 24) in comparison to control samples. The data were obtained using NanoString® technology. The data showed that OSMRβ mRNA levels are increased in subjects who have LP.

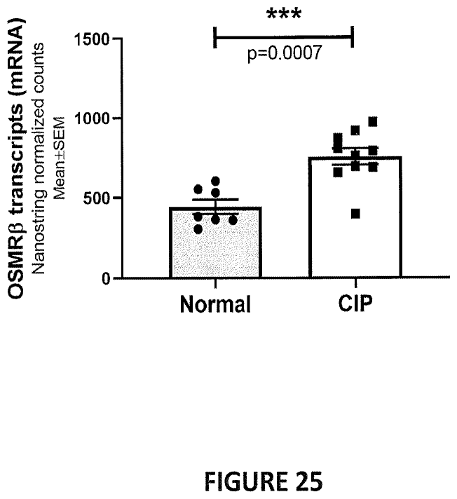

Furthermore, data were also obtained from skin samples isolated from subjects who have Chronic Idiopathic Pruritus (CIP) in comparison to control samples. The data were obtained using NanoString® technology. The data showed that OSMRβ mRNA levels are increased in subjects who have CIP (FIG. 25).

Collectively, these data indicate that targeting OSMRβ using an antibody as described herein is also useful for the treatment of LSC, LP, and/or CIP.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating prurigo nodularis (PN), comprising a step of: administering to a subject in need of treatment an anti-OSMRβ antibody at a therapeutically effective dose and an administration interval for a treatment period sufficient to improve, stabilize or reduce one or more symptoms of prurigo nodularis relative to a control.

2.-4. (canceled)

5. The method according to claim 1, wherein the prurigo nodularis is associated with one or more underlying co-morbidities.

6. The method according to claim 1, wherein IL-31 expression level is elevated in the subject relative to a control.

7. The method according to claim 1, wherein IL-31Rα expression level is elevated in the subject relative to a control.

8. The method according to claim 1, wherein OSM expression level is elevated in the subject relative to a control.

9. The method according to claim 1, wherein OSMRβ expression level is elevated in the subject relative to a control.

10. (canceled)

11. The method according to claim 6, wherein the control is a healthy subject, who is not diagnosed with a pruritic disease.

12.-27. (canceled)

28. A method of treating inflammation, the method comprising administering to a subject in need of treatment an anti-OSMRβ antibody at a therapeutically effective dose and an administration interval for a treatment period such that one or more symptoms associated with inflammation are reduced in intensity, severity, or frequency or has delayed in onset.

29. The method of claim 28, wherein the inflammation is T_{H}2 mediated inflammation.

30. The method according to claim 28, wherein the inflammation is independent of IL-31.

31.-34. (canceled)

35. A method of treating atopic dermatitis, comprising a step of:
administering to a subject in need of treatment an anti-OSMRβ antibody at a therapeutically effective dose and an administration interval for a treatment period sufficient to improve, stabilize or reduce one or more symptoms of atopic dermatitis relative to a control.

36. The method of claim 35, wherein the step of administering comprises subcutaneous or intravenous administration.

37.-40. (canceled)

41. The method according to claim 35, wherein the therapeutically effective dose is an initial loading dose, and wherein the method further comprises administering at least one maintenance dose.

42.-47. (canceled)

48. The method according to claim 35, wherein the therapeutically effective dose is a flat dose.

49. The method of claim 48, wherein the flat dose is equal to or greater than 50 mg, 100 mg, 150 mg, 200 mg, 250 mg, 300 mg, 320 mg, 360 mg, 380 mg, 400 mg, 450 mg, 500 mg, 550 mg, 600 mg, 650 mg, 700 mg, 720 mg, 740 mg, 760 mg, 780 mg or 800 mg.

50.-59. (canceled)

60. The method according to claim 35, wherein the one or more symptoms of atopic dermatitis are assessed by an Investigators' Global Assessment (IGA) of atopic dermatitis; Eczema Area and Severity Index (EASI); SCORing Atopic Dermatitis; Area Photographs; Body Surface Area Involvement (BSA); Dermatology Life Quality Index, (DLQI); Hospital Anxiety Depression Scale (HADS); actigraphy or quantitative numerical pruritus scale.

61.-133. (canceled)

134. The method according to claim 1, wherein the anti-OSMRβ antibody comprises:
a light chain complementary-determining region 1 (LCDR1) defined by SEQ ID NO: 8, a light chain complementary-determining region 2 (LCDR2) defined by SEQ ID NO: 9, and a light chain complementary-determining region 3 (LCDR3) defined by SEQ ID NO: 10; and
a heavy chain complementary-determining region 1 (HCDR1) defined by SEQ ID NO: 5, a heavy chain complementary-determining region 2 (HCDR2) defined by SEQ ID NO: 6, and a heavy chain complementary-determining region 3 (HCDR3) defined by SEQ ID NO: 7.

135. The method of claim 134, wherein the anti-OSMRβ antibody comprises: a light chain variable domain having an amino acid sequence at least 90% identical to SEQ ID NO: 4; and a heavy chain variable domain having an amino acid sequence at least 90% identical to SEQ ID NO: 3.

136. The method of claim 135, wherein the light chain variable domain has the amino acid sequence set forth in SEQ ID NO: 4; and the heavy chain variable domain has the amino acid sequence set forth in SEQ ID NO: 3.

137.-177. (canceled)

178. The method of according to claim 35, wherein the anti-OSMRβ antibody is administered in conjunction with an additional therapeutic agent.

179. (canceled)


SEQUENCE LISTING (16 sequences)

SEQ ID NO: 1 - protein, 452 aa, synthetic construct
  REGION 1..452: note = Synthetic polypeptide
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 216 aa, synthetic construct
  REGION 1..216: note = Synthetic polypeptide
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 126 aa, synthetic construct
  REGION 1..126: note = Synthetic polypeptide
QVQLVQSGAEVKKPGASVKVSCKASGYTFTSYEINWVRQATGQGLEWMGWMNPNSGYTGYAQKFQGRVTMTRDTSISTAYMEMSSLRSEDTAVYYCARDIVAANTDYYFYYGMDVWGQGTTVTVSS

SEQ ID NO: 4 - protein, 111 aa, synthetic construct
  REGION 1..111: note = Synthetic polypeptide
QSVLTQPPSASGTPGQRVTISCSGSNSNIGSNTVNWYHQLPGTAPKLLIYNINKRPSGVPDRFSGSKSGSSASLAISGLQSEDEADYYCSTWDDSLDGVVFGGGTKLTVLG

SEQ ID NO: 5 - protein, 5 aa, synthetic construct
  REGION 1..5: note = Synthetic oligopeptide
SYEIN

SEQ ID NO: 6 - protein, 21 aa, synthetic construct
  REGION 1..21: note = Synthetic oligopeptide
WMGWMNPNSGYTGYAQKFQGR

SEQ ID NO: 7 - protein, 17 aa, synthetic construct
  REGION 1..17: note = Synthetic oligopeptide
DIVAANTDYYFYYGMDV

SEQ ID NO: 8 - protein, 13 aa, synthetic construct
  REGION 1..13: note = Synthetic oligopeptide
SGSNSNIGSNTVN

SEQ ID NO: 9 - protein, 7 aa, synthetic construct
  REGION 1..7: note = Synthetic oligopeptide
NINKRPS

SEQ ID NO: 10 - protein, 11 aa, synthetic construct
  REGION 1..11: note = Synthetic oligopeptide
STWDDSLDGVV

SEQ ID NO: 11 - protein, 19 aa, synthetic construct
  REGION 1..19: note = Synthetic oligopeptide
MDFGLSLVFLVLILKGVQC

SEQ ID NO: 12 - protein, 26 aa, synthetic construct
  REGION 1..26: note = Synthetic oligopeptide
MATGSRTSLLLAFGLLCLSWLQEGSA

SEQ ID NO: 13 - protein, 220 aa, synthetic construct
  REGION 1..220: note = Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 14 - protein, 106 aa, synthetic construct [sequence omitted]

SEQ ID NO: 15 - protein, 326 aa, synthetic construct
  REGION 1..326: note = Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 105 aa, synthetic construct
  REGION 1..105: note = Synthetic polypeptide
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS